(19) United States
(12) Patent Application Publication
(10) Pub. No.: US 2024/0294670 A1
(43) Pub. Date: Sep. 5, 2024

(54) ANTIBODIES TO CHITINASE 3-LIKE-1 AND METHODS OF USE THEREFOR

(71) Applicant: THE BOARD OF REGENTS OF THE UNIVERSITY OF TEXAS SYSTEM, Austin, TX (US)

(72) Inventors: Cynthia JU, Bellaire, TX (US); Jongmin JEONG, Houston, TX (US); Zhao SHAN, Houston, TX (US); Leike LI, Houston, TX (US); Ningyan AN, Houston, TX (US); Zhiqiang AN, Houston, TX (US)

(73) Assignee: THE BOARD OF REGENTS OF THE UNIVERSITY OF TEXAS SYSTEM, Austin, TX (US)

(21) Appl. No.: 17/996,854

(22) PCT Filed: Apr. 21, 2021
(86) PCT No.: PCT/US2021/028338

§ 371 Date: Oct. 21, 2022

Related U.S. Application Data
(60) Provisional application No. 63/013,087, filed on Apr. 21, 2020.

(51) Int. Cl. C07K 16/40; A61K 39/00; A61K 45/06; A61P 35/00

(52) U.S. Cl. CPC C07K 16/40; A61K 39/4631; A61K 45/06; A61P 35/00; A61K 2039/505; A61K 2239/13; C07K 2317/11; C07K 2317/24; C07K 2317/31; C07K 2317/52; C07K 2317/565

(57) ABSTRACT

Provided herein are antibodies binding to CHI3L1 and the uses of the antibodies in detecting and treating cancer and for treating hepatotoxicity.

### PRIORITY CLAIM

This application claims benefit of priority to U.S. Provisional Application Ser. No. 63/013,087, filed Apr. 21, 2020, the entire contents of which are hereby incorporated by reference.

### STATEMENT REGARDING FEDERALLY FUNDED RESEARCH

The invention was made with government support under Grant No. DK122708 awarded by the National Institutes of Health. The government has certain rights in the invention.

### BACKGROUND

### 1. Field

The present disclosure relates generally to the fields of medicine, oncology, and immunology. More particular, the disclosure relates to antibodies that bind to chitinase 3-like-1 (CHI3L1) and can treat cancers, including hepatocellular carcinoma and metastatic breast cancer, and also treat acute liver injury.

### 2. Description of Related Art

Acute liver failure (ALF) is a life-threatening condition of massive hepatocyte injury and severe liver dysfunction that can result in multi-organ failure and death.¹ Thrombocytopenia is a common manifestation observed in patients with ALF.² It is reported that more than 60% of patients with ALF have a platelet count lower than 150,000 cells per cubic millimeter in circulation.³ Acetaminophen (APAP) overdose represents the most frequent cause of ALF in Western countries, which results in death or liver transplantation in more than one-third of patients.1 In patients with APAP overdose, thrombocytopenia is often observed.^{2,4-6} Thrombocytopenia is also described in mice treated with APAP and it correlates with hepatic accumulation of platelets, which critically contribute to APAP-induced liver injury (AILI).⁷ However, the underlying mechanism of the platelet recruitment into the liver during acute injury is poorly understood. Addressing this question may lead to the discovery of novel therapeutic targets. At present, the only antidote to treat AILI is N-acetylcysteine (NAC); however, its efficacy dramatically decreases when initiated more than a few hours after APAP overdose.⁸

The interaction between sinusoidal endothelial cells (LSEC) and platelet in injured liver has long been recognized.⁹⁻¹¹ However, emerging evidence suggests an indispensable role for Kupffer cells (KCs) in hepatic platelet recruitment during bacterial infection and nonalcoholic steatohepatitis (NASH).^{12,13} The current study uncovered a critical role of KCs, through the Chitinase 3-like-1/CD44 axis, in promoting hepatic platelet recruitment during acute liver injury. Chitinase 3-like-1 (CHI3L1, YKL-40 in humans) is a chitinase-like soluble protein without chitinase activities.¹⁴⁻¹⁶ It is produced by multiple cell types, including macrophages, neutrophils, fibroblasts, synovial cells, endothelial cells, and tumor cells.^{15,17} CHI3L1 has been implicated in many biological processes including apoptosis, inflammation, oxidative stress, infection, and tumor metastasis.¹⁸ CD44 is a type I transmembrane glycoprotein expressed on many mammalian cells, including endothelial cells, epithelial cells, fibroblasts, keratinocytes and leukocytes.¹⁹ CD44 has been implicated in a number of pathological conditions including cancer, arthritis, diabetes, vascular disease, and infections.²⁰⁻²⁹

### SUMMARY

Thus, in one aspect, the present disclosure provides an isolated monoclonal antibody or an antigen-binding fragment thereof comprising cloned paired heavy and light chain CDRs from Table A. In some aspects, the antibody or fragment thereof is encoded by clone-paired heavy and light chain sequences from FIGS. 24, 25 and 29. In certain aspects, the antibody or fragment thereof is encoded by heavy and light chain variable sequences having at least 70%, 80%, 90% or 95% identity to clone-paired sequences from FIGS. 24, 25 and 29. In other aspects, the antibody or fragment thereof comprises clone-paired heavy and light chain sequences from FIGS. 26, 27 and 28. In further aspects, the antibody or fragment thereof comprises heavy and light chain variable sequences having at least 70%, 80%, 90% or 95% identity to clone paired sequences from FIGS. 26, 27 and 28. In specific aspects, the isolated monoclonal antibody is a murine, a rodent, or a rabbit. In particular aspects, the isolated monoclonal antibody is a humanized, or human antibody.

In further aspects, the antigen-binding fragment is a recombinant ScFv (single chain fragment variable) antibody, Fab fragment, F(ab′)2 fragment, or Fv fragment. In some aspects, the isolated monoclonal antibody is a bispecific antibody or a chimeric antibody. In certain aspects, said antibody is an IgG, or a recombinant IgG antibody or antibody fragment comprising an Fc portion mutated to alter (eliminate or enhance) FcR interactions, to increase half-life and/or increase therapeutic efficacy, such as a LALA, N297, GASD/ALIE, YTE or LS mutation or glycan modified to alter (eliminate or enhance) FcR interactions such as enzymatic or chemical addition or removal of glycans or expression in a cell line engineered with a defined glycosylating pattern.

In another embodiment, there is provided an isolated monoclonal antibody or an antigen binding fragment thereof, which competes for the same epitope with the isolated monoclonal antibody or an antigen-binding fragment thereof according to any of the embodiments and aspects described above.

In yet another embodiment, there is provided a pharmaceutical composition comprising the isolated monoclonal antibody or an antigen-binding fragment thereof according to any of the embodiments and aspects described above, and a pharmaceutically acceptable carrier.

In still a further embodiment, there is provided an isolated nucleic acid that encodes the isolated monoclonal antibody according to any of the embodiments and aspects described above. Another embodiment provides a vector comprising the isolated nucleic acid of this embodiment. Yet a further embodiment provides a host cell comprising the vector of said embodiment. In some aspects, the host cell is a mammalian cell. In other aspects, the host cell is a CHO cell.

In yet still a further embodiment, there is provided a hybridoma encoding or producing the isolated monoclonal antibody according to any of the embodiments and aspects described above. A further embodiment provides a process of producing an antibody, comprising culturing the host cell described above under conditions suitable for expressing the antibody, and recovering the antibody. In still a further embodiment, there is provided a chimeric antigen receptor (CAR) protein comprising an antigen-binding fragment according to any of the embodiments and aspects described above. Another embodiment provides an isolated nucleic acid that encodes the CAR protein as described above. In yet still a further embodiment, there is provided a vector comprising the isolated nucleic acid of this embodiment. A further embodiment provides an engineered cell comprising the isolated nucleic acid of said embodiment. In certain aspects, the cell is a T cell, NK cell, or macrophage.

In a further embodiment, there is provided a method of treating or ameliorating the effect of a cancer in a subject, the method comprising administering to the subject a therapeutically effective amount of the antibody or an antigen-binding fragment thereof according to any of embodiments and aspects described herein or the engineered cell described herein. In some aspects, the method reduces or eradicates the tumor burden in the subject. In certain aspects, the method reduces the number of tumor cells or reduces tumor size. In specific aspects, the method eradicates the tumor in the subject. In particular aspects, the cancer is a solid tumor cancer such as lung cancer, brain cancer, skin cancer, head and neck cancer, liver cancer, pancreatic cancer, stomach cancer, bladder cancer, colon cancer, testicular cancer, cervical cancer, breast cancer, or uterine cancer. In further aspects, the cancer is a hematologic malignancy, such as myelodysplastic syndromes, myeloproliferative neoplasms, chronic myelomonocytic leukemia (CMML), chronic myelocytic leukemia, or acute myeloid leukemia (AML), acute promyelocytic leukemia (APL) or M3 AML, acute myelomonocytic leukemia or M4 AML, acute monocytic leukemia or M5 AML, acute myeloblastic leukemia, or polycythemia vera. In some aspects, the antibody or an antigen-binding fragment thereof is administered intravenously, intra-arterially, intra-tumorally, or subcutaneously.

In additional aspects, the method further comprises administering to the subject a second anti-cancer therapy, such as one or more drugs selected from the group consisting of a topoisomerase inhibitor, an anthracycline topoisomerase inhibitor, an anthracycline, a daunorubicin, a nucleoside metabolic inhibitor, a cytarabine, a hypomethylating agent, a low dose cytarabine (LDAC), a combination of daunorubicin and cytarabine, a daunorubicin and cytarabine liposome for injection, Vyxeos®, an azacytidine, Vidaza®, a decitabine, an all-trans-retinoic acid (ATRA), an arsenic, an arsenic trioxide, a histamine dihydrochloride, Ceplene®, an interleukin-2, an aldesleukin, Proleukin®, a gemtuzumab ozogamicin, Mylotarg®, an FLT-3 inhibitor, a midostaurin, Rydapt®, a clofarabine, a farnesyl transferase inhibitor, a decitabine, an IDH1 inhibitor, an ivosidenib, Tibsovo®, an IDH2 inhibitor, an enasidenib, Idhifa®, a smoothened (SMO) inhibitor, a glasdegib, an arginase inhibitor, an IDO inhibitor, an epacadostat, a BCL-2 inihbitor, a venetoclax, Venclexta®, a platinum complex derivative, oxaliplatin, a kinase inhibitor, a tyrosine kinase inhibitor, a PI3 kinase inhibitor, a BTK inhibitor, an ibrutinib, IMBRUVICA®, an acalabrutinib, CALQUENCE®, a zanubrutinib, a PD-1 antibody, a PD-L1 antibody, a CTLA-4 antibody, a LAG3 antibody, an ICOS antibody, a TIGIT antibody, a TIM3 antibody, a CD40 antibody, a 4-1BB antibody, a CD47 antibody, a SIRP1α antibody or fusions protein, an antagonist of E-selectin, an antibody binding to a tumor antigen, an antibody binding to a T-cell surface marker, an antibody binding to a myeloid cell or NK cell surface marker, an alkylating agent, a nitrosourea agent, an antimetabolite, an antitumor antibiotic, an alkaloid derived from a plant, a hormone therapy medicine, a hormone antagonist, an aromatase inhibitor, and a P-glycoprotein inhibitor.

In certain aspects, isolated monoclonal antibody or an antigen binding fragment thereof further comprises an antitumor drug linked thereto. In a further aspect, said antitumor drug is linked to said antibody through a photolabile linker. In another aspect, said antitumor drug is linked to said antibody through an enzymatically-cleaved linker. In some aspects, said antitumor drug is a toxin, a radioisotope, a cytokine, or an enzyme.

In a further embodiment, there is provided a method of detecting a cancer cell or cancer stem cell in a sample or subject comprising:
(a) contacting a subject or a sample from the subject with the antibody or an antigen-binding fragment thereof according to any of the embodiments and aspects described above; and
(b) detecting binding of said antibody to a cancer cell or cancer stem cell in said subject or sample.

In specific aspects, the sample is a body fluid or biopsy. In other aspects, the sample is blood, bone marrow, sputum, tears, saliva, mucous, serum, urine or feces. In some aspects, detection comprises immunohistochemistry, flow cytometry, FACS, ELISA, RIA or Western blot. In certain aspects, the method further comprises performing steps (a) and (b) a second time and determining a change in detection levels as compared to the first time. In additional aspects, said isolated monoclonal antibody or an antigen binding fragment thereof further comprises a label. In further aspects, said label is a peptide tag, an enzyme, a magnetic particle, a chromophore, a fluorescent molecule, a chemo-luminescent molecule, or a dye. In another aspect, said isolated monoclonal antibody or an antigen binding fragment thereof is conjugated to a liposome or nanoparticle

In still yet a further embodiment, there is provided a method of treating a subject having or at risk of hepatotoxicity comprising administering an antibody according to any of the embodiments and aspects described above to said subject. In some aspects, the hepatotoxicity is due to acute liver failure. In other aspects, the hepatotoxicity is due to a medicinal agent or drug, such as acetaminophen, a laboratory chemical, an agricultural chemical, such as a herbicide or pesticide, an industrial chemical, or natural product, such as a plant toxin. In certain aspects, the medicinal agent or drug is dosed at a therapeutic level. In particular aspects, the medicinal agent or drug is dosed above a therapeutic level, i.e., is an overdose. In some specific aspects, the antibody is administered more than one, such as daily, every other day, twice a week or weekly. In additional aspects, the method further comprises administering to said subject a second hepatotoxicity therapy, such as fluids, pain medicine, anti-toxin. In certain aspects, said subject has been diagnosed with hepatoxicity. In other aspects, said subject is suspected of having induced or contacted an hepatoxic agent or dose of an agent. In another aspect, the method further comprises assessing liver function and/or liver enzymes before and/or after administering said antibody.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-E. Hepatic platelet accumulation and their contribution to AILI. (FIG. 1A) IHC staining to detect platelets (CD41+) in normal liver biopsies (Normal) and those from patients with AILI (Patient). The numbers of intrahepatic platelets were quantified (n=10/group). Scale bar, 250 μm. (FIG. 1B) IF staining for intrahepatic platelets (CD41+) and LSECs (CD31+) in Male WT mice treated with PBS or APAP for 3 h. The numbers of intrahepatic platelets were quantified (n=6/group). Scale bar, 25 μm. (FIGS. 1C-D) Mice were treated with control IgG (Ctrl IgG) or an anti-CD41 antibody (α-CD41 Ab) either 3 h before or 3 h after APAP administration. At 24 h post-APAP treatment, serum ALT levels were determined (FIG. 1C), and liver histology evaluated with necrotic areas outlined (FIG. 1D). Scale bar, 250 μm. (FIG. 1E) CD41 staining of intrahepatic platelets of mice treated in FIG. 1C. (n=5 mice/group in FIGS. 1C-E. Two-tailed, unpaired student t-test was performed in FIGS. 1A-C).

FIGS. 2A-I. CHI3L1 mediates the function of KCs in promoting hepatic platelet accumulation. (FIG. 2A) IHC staining for macrophages (CD68+) and platelets (CD41+) in normal liver biopsies (Normal) and those from patients with AILI (Patient) (n=10/group). Scale bar, 25 μm. (FIG. 2B) IF staining for intrahepatic platelets (CD41+) and KCs (F4/80+) in WT mice treated with PBS or APAP for 3 h. Scale bar, 25 μm. Arrowheads indicate platelets adherent to KCs. (FIG. 2C) Quantification of platelets adherent to KCs or LSECs. (FIG. 2D) IF staining for intrahepatic platelets (CD41+) and KCs (F4/80+) in WT mice treated with PBS-containing empty liposome (PBS) or clodronate-containing liposome (CLDN) for 9 h following by APAP treatment for another 6 h. Scale bar, 25 μm. (FIGS. 2E-F) Mice were treated as described in d, serum ALT levels were determined and liver histology evaluated with necrotic areas outlined. (n=6 mice/group in (FIGS. 2B-F). Scale bar, 250 μm. (FIG. 2G) IHC detection and quantification of intrahepatic macrophages (CD68+) in normal liver biopsies (Normal) and those from patients with AILI (Patient) (n=10/group). Scale bar, 250 μm. (FIG. 2H) ELISA analysis of macrophage activation factors in liver homogenates from WT mice treated with PBS- or APAP for 2 h (n=4 mice/group). (FIG. 2I) IF staining to detect intrahepatic platelets (CD41+) and KCs (F4/80+). The numbers of platelets were quantified in naïve CHI3L1^{-/-} mice and APAP-treated CHI3L1^{-/-} mice with additional treatment of either PBS or rCHI3L1 at 3 h prior to APAP challenge (n=3-4 mice/group). Scale bar, 25 μm. Two-tailed, unpaired student t-test was performed in FIGS. 2C, 2E, and 2H. One-way ANOVA were performed in FIG. 2I.

FIGS. 3A-D. Chi31 interacts with CD44 on KCs. (FIG. 3A) Immunoprecipitation with anti-CD44 antibody was performed using liver homogenates obtained from WT and CD44^{-/-} mice treated with APAP for 2 h. Input proteins and immune-precipitated proteins were blotted with the indicated antibodies. (FIG. 3B) Interferometry measurement of the binding kinetics of human His-CHI3L1 with human Fc-CD44. (FIG. 3C) His-tagged control and human CHI3L1 were incubated with recombinant human CD44. Proteins bound to CHI3L1 were immunoprecipitated with an anti-His antibody. Input proteins and immune-precipitated proteins were blotted with indicated antibodies. (FIG. 3D) Flow cytometry analysis to identify rCHI3L1-binding cells among liver non-parenchymal cells isolated from WT mice treated with APAP for 2 h. CD44+ cells were gated from CD45+ single live cells. rCHI3L1-binding cells were further gated from CD44+ cells. The CHI3L1+CD44+ cells were then identified by specific markers that recognize various cell types, such as F4/80 (KCs), CD146 (LSECs), Ly6C (monocytes).

FIGS. 4A-F. CHI3L1 promotes platelet recruitment and liver injury through CD44. (FIG. 4A), Male WT mice were treated with APAP. Male CHI3L1^{-/-} and CD44^{-/-} mice were treated with PBS or rCHI3L1 plus APAP (n=3 mice/group). Mice were sacrificed at 3 h post-APAP treatment and IF staining was performed to detect intrahepatic platelets (CD41+). Liver sinusoids were outlined by CD31 staining for LSEC. Scale bar, 25 μm. (FIGS. 4B, 4C) Mice were treated as described in panel a and sacrificed at 24 h post-APAP challenge. Liver histology was evaluated with necrotic areas outlined (n=6 mice/group), and serum ALT activities were measured (n=6-10 mice/group). Scale bar, 250 μm. (FIG. 4D) CHI3L1^{-/-} mice reconstituted with rCHI3L1 were treated with either Ctrl IgG or α-CD44 Ab 30 min prior to APAP challenge. Liver sections were harvested at 3 h post-APAP treatment (n=3 mice/group). IF staining was performed to detect intrahepatic platelets (CD41+). Liver sinusoids were outlined by CD31 staining for LSECs. Scale bar, 25 μm. (FIGS. 4E, 4F) CHI3L1^{-/-} mice were treated as described in FIG. 4D and sacrificed at 24 h post-APAP challenge. Liver histology was evaluated with necrotic areas outlined and serum ALT activities were measured (n=4-5 mice/group). Scale bar, 250 μm. One-way ANOVA were performed in FIG. 4C. Two-tailed, unpaired student t-test was performed in FIG. 4F.

FIGS. 5A-H. Podoplanin, regulated by the CHI3L1/CD44 axis, contributes to KCs-mediated platelets recruitment. (FIG. 5A) mRNA levels of podoplanin (PDPN) were analyzed by qPCR in KCs, LSECs, hepatic stellate cells (HSC) and hepatocytes (Hep) isolated from naïve WT mice and WT mice treated with APAP for 1 h and 3 h (n=4 mice/group). (FIG. 5B) WT mice were treated with APAP. CHI3L1^{-/-} and CD44^{-/-} mice were treated with PBS or rCHI3L1 followed by APAP challenge 3 h later (n=3 mice/group). Mice were sacrificed at 3 h post-APAP and KCs isolated. mRNA levels of PDPN in KCs were analyzed by qPCR. (FIGS. 5C, 5D) WT, CHI3L1^{-/-} and CD44^{-/-} mice were treated as described in FIG. 5B. IF staining of liver sections for PDPN and F4/80 is shown FIG. 5C and the proportions of KCs that express PDPN were quantified FIG. 5D. Scale bar, 25 μm. (FIG. 5E) CHI3L1^{-/-} mice reconstituted with rCHI3L1 were treated with either Ctrl IgG or α-podoplanin Ab for 16 h and subsequently challenged with APAP (n=3 mice/group). Mice were sacrificed at 3 h post-APAP treatment and IF staining was performed to identify intrahepatic platelets (CD41+) and KCs (F4/80+). Scale bar, 25 μm. (FIGS. 5F, 5G) Mice were treated as described in panel E and sacrificed at 24 h post-APAP challenge. Liver histology was evaluated with necrotic areas outlined, and serum ALT activities were measured (n=6 mice/group). Scale bar, 250 μm. (FIG. 5H) KCs were isolated from WT mice treated with APAP for 3 h. The cells were treated in vitro with either a control IgG (Ctrl IgG) or α-podoplanin (PDPN) Ab before incubated with platelets. IF staining was performed to detect PDPN on KCs and Clec-2 on platelets. Scale bar, 25 μm. One-way ANOVA were performed in FIGS. 5A, 5B, and 5D. Two-tailed, unpaired student t-test was performed in FIG. 5G.

FIGS. 6A-H. Therapeutic potential of targeting CHI3L1 to treat AILI. (FIG. 6A) Male WT mice were treated with APAP for 3 h, and then injected (i.p.) with either a control IgG (Ctrl IgG) or an anti-mouse CHI3L1 Ab (α-mCHI3L1 Ab, C59). IF staining for intrahepatic platelets (CD41+) was performed at 6 h post-APAP (n=3 mice/group). Scale bar, 25 μm. (FIGS. 6B, 6C) Mice were treated as described in FIG. 6A and sacrificed at 24 h post-APAP challenge. Liver histology was evaluated with necrotic areas outlined and serum ALT activities were measured (n=4-6 mice/group). (FIG. 6D) IHC staining for CHI3L1 in normal liver biopsies (Normal) and those from patients with AILI (Patient) (n=10/group). Scale bar, 250 μm. (FIG. 6E) CHI3L1^{-/-} mice were treated with APAP for 3 h and then divided into several sub-groups that were treated with PBS, recombinant human CHI3L1 (rhCHI3L1), rhCHI3L1 plus a control IgG (Ctrl IgG) or rCHI3L1 plus an anti-human CHI3L1 Ab (α-hCHI3L1 Ab, C7). IF staining was performed to identify intrahepatic platelets (CD41+) at 6 h post-APAP. Scale bar, 25 μm. (FIGS. 6F, 6G) CHI3L1^{-/-} mice were treated as described in FIG. 6E and sacrificed at 24 h post-APAP challenge. (FIG. 6H) Liver histology was evaluated with necrotic areas outlined, and serum ALT activities were measured (n=6 mice/group). Scale bar, 250 μm. (n=5-10 mice/group in (FIGS. 6E-G). Two-tailed, unpaired student t-test was performed in FIG. 6C. One-way ANOVA were performed in FIG. 6G.

FIG. 7. The ELISA binding of the supernatant antibody to the human CHI3L1. (hCHI3L1), and mouse CHI3L1 (mCHI3L1). The CHI3L1 protein was coated on maxi-sorp 96-well plates, cell culture supernatant (100 ul/well) was used for binding on the antigen coated plate. Binding signals (A450 nm) are shown in Y-axis and CHI3L1 mAbs are shown on X-axis.

FIG. 8. Kinetic binding sensorgrams collected on Octet instrument for determination of kon and koff rates for each CHI3L1 antibody. The individual mAbs were captured on a protein A sensor and the CHI3L1 concentrations used in each curve (from low to high) are from 3.55, 7.1, 14.2, 28.4, 56.8 nM, respectively.

FIGS. 9A-D. CHI3L1 mAb (C9) treatment of breast cancer tumor growth in vivo using 4T1 syngeneic mouse tumor model. (FIG. 9A) Tumor growth curve for CHI3L1mAb (Tx CHI3L1) treated group in comparison with the control (Tx PBS). The error bars indicate standard deviation (SD) and n=5. (FIG. 9B) Individual mouse growth curve and red lines (Mouse #-CHI3L1) for CHI3L1 mAb treated group and the blank lines are for control group. (FIG. 9C) CHI3L1 mAb treated mice (Tx CHI3L1) showed significantly reduced tumor weights than that in control group (Tx PBS), n=5. (FIG. 9D) Tumor images were taken after scarifying mice and tumors were removed from each mouse and two groups of tumor images from control mice are shown.

FIG. 10. Inhibition of cancer cell migration by treatment with CHI3L1 mAbs (10 μg/ml) using a scratching assay. The blank indicates no antibody treatment control and scratch gap was filled with cancer cells. Treatment with C59, C68, C75, and C79 mAbs showed inhibition of cell migration as indicated the unfilled gaps indicated by the red arrow.

FIG. 11. C #59 ab (α-CHI3L1) attenuates hepatic tumor development.

FIG. 12. Tumor development is ameliorated in CHI3L1 KO mice.

FIG. 13. Inhibition of rCHI3L1-induced HCC cell (Hepa-16) migration by the treatment of anti-CHI3L1 mAb (C59, 10 μg/mL) in a scratch assay.

FIG. 14. Inhibition of rCHI3L1-induced angiogenesis by the treatment of anti-CHI3L1 mAb (C59, 10 μg/mL) in a tube formation assay of a mouse endothelial cell line (TSEC).

FIGS. 15A-B. TCGA database showing increased Chi3l1 mRNA in HCC and its correlation with survival. (FIG. 15A) Based on Cancer Genome Atlas (TCGA) database, Chi3l1 transcript levels (mRNA expression) of healthy controls and HCC patients were analyzed. (FIG. 15B) The overall survival rates (%) in patients were compared between two groups with high and low Chi3l1 mRNA expression levels.

FIGS. 16A-B. Chi3l1 is elevated in the sera and tumors from HCC patients. (FIG. 16A) Serum samples were collected from HCC patients with different etiologies (Hepatitis B virus; HBV, Hepatitis C virus; HCV, Non-alcoholic steatohepatitis; NASH, Alcoholic steatohepatitis; ASH). Protein levels of Chi3l1 in sera were measured by ELISA. (FIG. 16B) Chi3l1 expression in tumors and non-tumor adjacent liver tissues from HCC patients were detected by IHC staining.

FIGS. 17A-B. Neutralizing Chi3l1 by C59mAb inhibits HCC progression in vivo. (FIG. 17A) Mice were implanted with haptic tumor cells (Hepa1-6 cells) directly into the liver. After 1 week, mice were randomly were divided into two groups and treated with IgG or C59 mAb (200 μg per mouse, twice per week) for 4 weeks. (FIG. 17B) Mice were hydrodynamically injected through the tail vein oncogenes (b-catenin, c-Met) together with sleeping beauty (SB) plasmids. After 4 weeks, mice were treated with C59 mAb (200 μg per mouse, twice per week) for 4 weeks.

FIGS. 18A-D. Effects of Chi3l1 directly on tumor cell proliferation, apoptosis, migration and invasion. Hepa1-6 cells were treated with rChi3l1 protein (1 μg/ml) with or without C59mAb (10 μg/ml) for 24 h, then analyzed for cell proliferation using WST-1 reagent (FIG. 18A), migration and invasion using 8 μm insert (FIG. 18B). and collagen coating matrix (FIG. 18C). To induce apoptosis, the cells were treated with a high concentration of palmitic acid (PA, 400 μM) and rChi3l1 protein (1 μg/ml) with or without C59mAb (10 μg/ml). After 24 hr, cells were analyzed for annexin V (ANX V) expression using flow cytometry (FIG. 18D).

FIG. 19. Tumor growth is significantly reduced in Chi3l1−/− mice. Chi3l1−/− and WT Mice were implanted with haptic tumor cells (Hepa1-6 cells) directly onto the liver. After 5 weeks, mice were sacrificed to measure tumor size.

FIG. 20. Chi3l1 is expressed by tumor-associated macrophages (TAMs). Mice were treated as described in FIGS. 17A-B. Chi3l1 and F4/80 expression in the liver were detected by IHC staining.

FIG. 21. C59mAb treatment restored a pro-inflammatory and anti-tumorigenic phonotype of TAMs. Mice were treated as described in FIGS. 17A-B. Total liver mononuclear cells (MNCs) were isolated by gradient-dependent centrifugation. Hepatic macrophages were isolated by magnetic-associated cell sorting using anti-CD11b and anti-F4/80 antibodies. Isolated hepatic macrophages were analyzed for levels of mRNA expression by qPCR analysis.

FIGS. 22A-B. C59mAb treatment reduces Arg1 expression by TAMs. Mice were treated as described in FIGS. 17A-B. F4/80 and Arg1 expression in the liver were detected by IHC staining (FIG. 22A). Liver MNCs were isolated from the tumors and analyzed by flow cytometry. CD11b+Ly6G⁻ F4/80⁺MHC II⁺ cells were gated. Arg1 expression was shown (FIG. 22B).

FIGS. 23A-C. The binding affinities of C59Hu and C7Hu detected using Bio-Layer Interferometry. (FIG. 23A) C59Hu binding to mouse Chi3L1. (FIG. 23B) C59Hu binding to human Chi3L1. (FIG. 23C) C7Hu binding to human Chi3L1.

FIG. 24. Heavy chain (HC) variable DNA sequences of anti-Chil3 antibodies.

FIG. 25. Light chain (LC) variable DNA sequences of anti-Chil3 antibodies.

FIG. 26. Heavy chain (HC) variable amino acid sequences of anti-Chil3 antibodies.

FIG. 27. Light chain (LC) variable amino acid sequences of anti-Chil3 antibodies.

FIG. 28. Humanized C59 and C7 amino acid sequences.

FIG. 29. Humanized C59Hu and C7Hu DNA sequences.

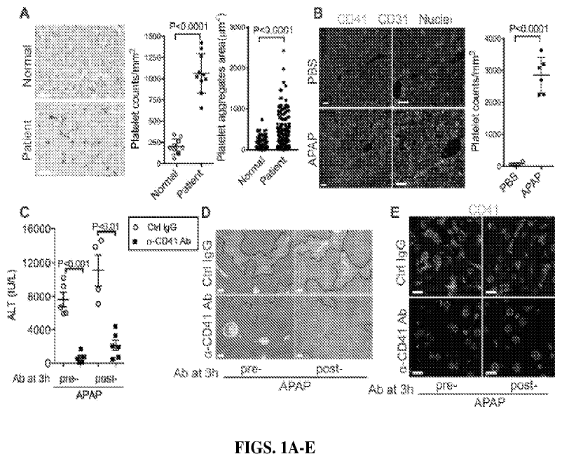

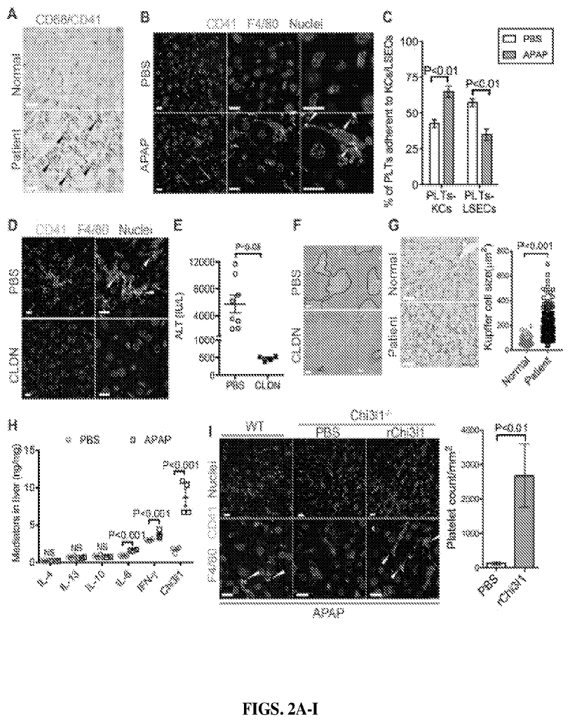

EXTENDED DATA FIG. 1. KCs were depleted at 15 h post CLDN treatment. Male WT mice were injected with either PBS-containing empty liposome (PBS) or clodronate-containing liposome (CLDN) for 9 h following by APAP treatment for another 6 h. NPCs were isolated and underwent flow cytometry analysis. Indicated cells were gated on single live CD146− cells.

EXTENDED DATA FIGS. 2A-H. KCs rapidly recruit platelets to exacerbate liver injury in Con A-induced liver injury (CILI). (ED FIG. 2A), IF staining for intrahepatic platelets (CD41+) in Male WT mice treated with PBS or Con A for 6 h. The numbers of intrahepatic platelets were quantified (n=3/group). Scale bar, 25 μm. (ED FIGS. 2B, 2C) Male WT Mice were treated with control IgG (Ctrl IgG) or an anti-CD41 antibody (α-CD41 Ab) 12 h before Con A administration. At 24 h post-Con A treatment, serum ALT levels were determined and liver histology evaluated with necrotic areas outlined. Scale bar, 250 μm (n=5 mice/group). (ED FIG. 2D) CD41 staining of intrahepatic platelets of mice treated in b at 24 h post-Con A treatment. (n=3 mice/group). (ED FIG. 2E) IF staining for intrahepatic platelets (CD41+) and KCs (F4/80+) in WT mice treated with PBS or Con A for 6 h. Scale bar, 25 μm. Arrowheads indicate platelets adherent to KCs (n=3 mice/group). (ED FIG. 2F) IF staining for intrahepatic platelets (CD41+) and KCs (F4/80+) in WT mice treated with PBS-containing empty liposome (PBS) or clodronate-containing liposome (CLDN) for 9 h following by Con A treatment for another 6 h. Scale bar, 25 μm. (ED FIGS. 2G, 2H) WT Mice were treated as described in f and sacrificed at 6 h after Con A challenge. Serum ALT levels were determined and liver histology evaluated with necrotic areas outlined. Scale bar, 250 μm. (n=4 mice/group in ED FIGS. 2G and 2H). Two-tailed, unpaired student t-test was performed in ED FIGS. 2A, 2B and 2G.

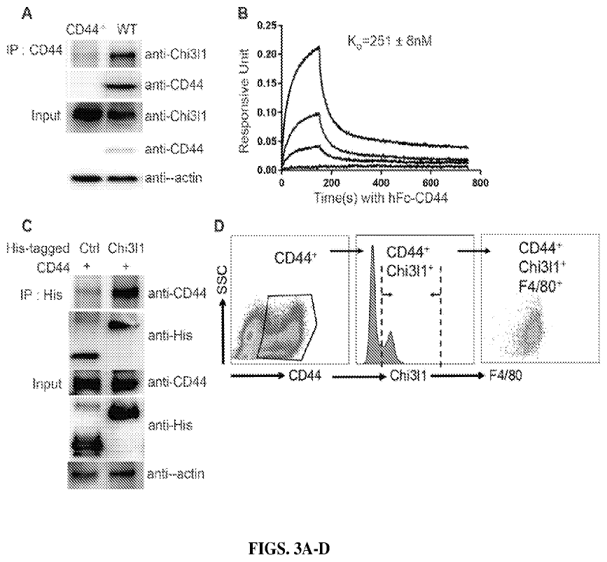

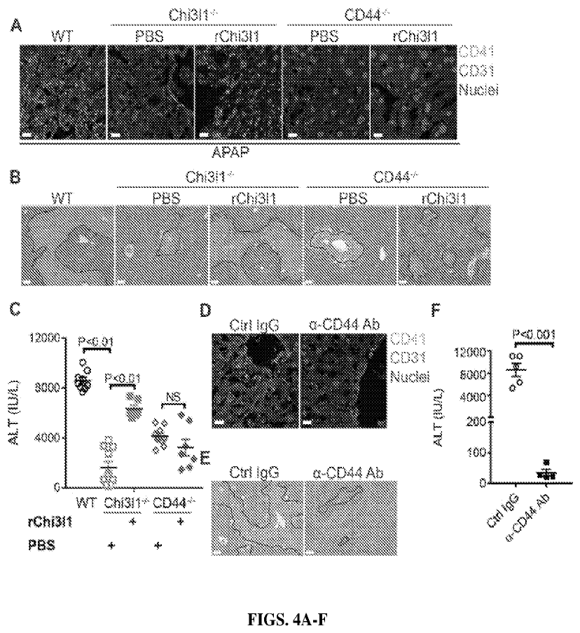

EXTENDED DATA FIGS. 3A-C. CHI3L1- or CD44-deletion does not affect APAP bio-activation. Male WT mice were treated with APAP. (ED FIG. 3A) GSH levels in the liver were measured at indicated time points by HPLC. (ED FIG. 3B) Hepatic protein levels of CYP2E1 were measured by Western blotting after mice were fasted overnight but prior to APAP treatment. (ED FIG. 3C) NAPQI-protein adducts in liver were measured by Western blotting at 2 h post-APAP treatment. (n=3 mice/group).

EXTENDED DATA FIGS. 4A-E. CHI3L1 promotes hepatic platelet recruitment and liver injury through CD44 in CILI. (ED FIG. 4A) Male WT mice were treated with Con A. Male CHI3L1^{-/-} and CD44^{-/-} mice were treated with PBS or rCHI3L1 plus Con A (n=3 mice/group). Mice were sacrificed at 6 h post-Con A treatment and IF staining was performed to detect intrahepatic platelets (CD41+). Scale bar, 25 μm. (ED FIGS. 4B, 4C) Male WT mice were treated as described in panel a and sacrificed at 24 h post-Con A challenge. Liver histology was evaluated with necrotic areas outlined and serum ALT activities were measured (n=4-5 mice/group). Scale bar, 250 μm. (ED FIG. 4D) CHI3L1^{-/-} mice reconstituted with rCHI3L1 were treated with either Ctrl IgG or α-CD44 Ab 30 min prior to Con A challenge. Liver sections were harvested at 3 h post-Con A treatment (n=3 mice/group). IF staining was performed to detect intrahepatic platelets (CD41+). Scale bar, 25 μm. (ED FIG. 4E) CHI3L1−/− mice reconstituted with rCHI3L1 were treated as described in ED FIG. 4D and sacrificed at 24 h post-APAP challenge. Liver histology was evaluated with necrotic areas outlined and serum ALT activities were measured (n=6 mice/group). Scale bar, 250 μm. Two-tailed, unpaired student t-test was performed in ED FIGS. 4D and 4E.

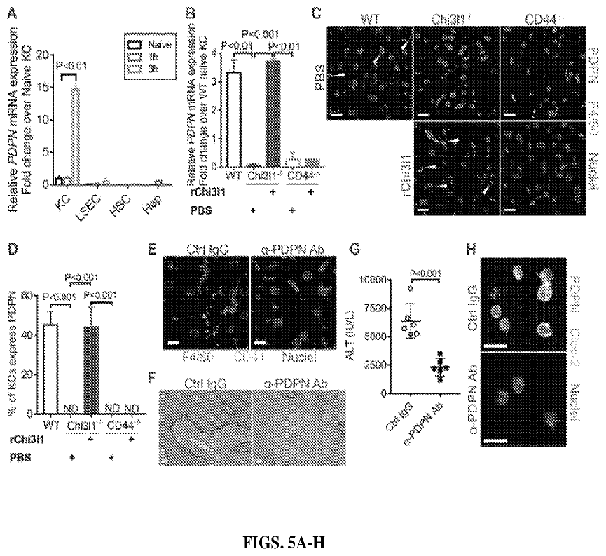

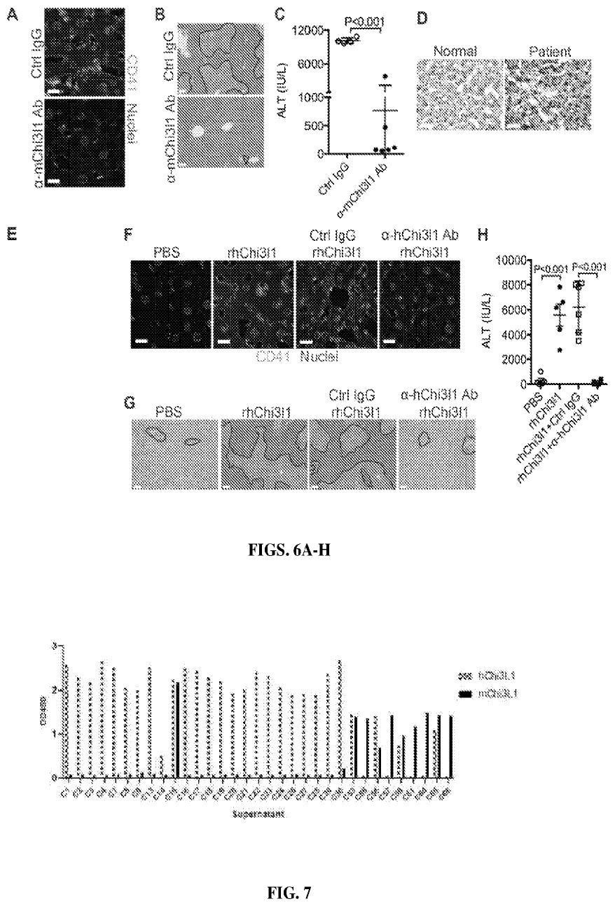

EXTENDED DATA FIG. 5. Measurement of mRNA levels of platelet adhesion molecules in KCs. Male WT, CHI3L1^{-/-}, CD44^{-/-} mice were treated with APAP (n=4 mice/group). After 3 h, mice were sacrificed and KCs were isolated to measure mRNA levels of various adhesion molecules, including P-Selectin Glycoprotein Ligand 1 (PSGL-1), Cluster of differentiation 40 (CD40), Cluster of differentiation 147 (CD147), Intercellular adhesion molecule 1 (ICAM), Lymphocyte function-associated antigen 1 (LFA1), von Willebrand Factor (VWF). One-way ANOVA were performed.

EXTENDED DATA FIG. 6. Examine the effectiveness of a panel of anti-mouse CHI3L1 monoclonal antibodies (anti-mCHI3L1 mAb) in attenuating AILI. Male WT mice were treated with APAP and after 3 h injected (i.p) with various clones of anti-mCHI3L1 mAb. Serum ALT levels were determined at 6 h and 24 h post-APAP treatment (n=3-6 mice/group). One-way ANOVA were performed.

EXTENDED DATA FIG. 7. Female CHI3L1^{-/-} and CD44^{-/-} mice develop reduced compared to female WT mice. Female WT, CHI3L1^{-/-} and CD44^{-/-} mice were treated with APAP. After 6 h and 24 h, serum ALT levels were measured (n=6-8 mice/group). One-way ANOVA were performed.

### DESCRIPTION OF ILLUSTRATIVE EMBODIMENTS

The present disclosure describes studies aimed at elucidating the underlying cellular and molecular mechanisms of hepatic platelet accumulation during acute liver injury using murine models of acetaminophen (APAP)-induced liver injury (AILI) and concanavalin A (Con A)-induced hepatitis. Moreover, the successful development of anti-CHI3L1 antibodies revealed that CHI3L1 is viable therapeutic target for the treatment of AILI by such neutralizing antibodies. In addition, based on the recently elucidates role for CHI3L1 in cancers, these same antibodies can also be used to treat cancer.

The following description of the disclosure is merely intended to illustrate various embodiments of the disclosure. As such, the specific modifications discussed are not to be construed as limitations on the scope of the disclosure. It will be apparent to one skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entirety.

### I. Definition

It is to be understood that both the foregoing general description and the following detailed description are exemplary and explanatory only and are not restrictive of the invention as claimed. In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise. Also, the use of the term “portion” can include part of a moiety or the entire moiety.

As used herein, the singular forms “a”, “an” and “the” include plural references unless the context clearly dictates otherwise.

The term “about” as used herein when referring to a measurable value such as an amount, a temporal duration, and the like, is meant to encompass variations of up to ±10% from the specified value. Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the disclosed subject matter. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

The term “antibody” refers to an intact immunoglobulin of any isotype, or a fragment thereof that can compete with the intact antibody for specific binding to the target antigen, and includes, for instance, chimeric, humanized, fully human, and bispecific antibodies. An “antibody” is a species of an antigen binding protein. An intact antibody will generally comprise at least two full-length heavy chains and two full-length light chains, but in some instances can include fewer chains such as antibodies naturally occurring in camelids which can comprise only heavy chains. Antibodies can be derived solely from a single source, or can be “chimeric,” that is, different portions of the antibody can be derived from two different antibodies as described further below. The antigen binding proteins, antibodies, or binding fragments can be produced in hybridomas, by recombinant DNA techniques, or by enzymatic or chemical cleavage of intact antibodies. Unless otherwise indicated, the term “antibody” includes, in addition to antibodies comprising two full-length heavy chains and two full-length light chains, derivatives, variants, fragments, and muteins thereof, examples of which are described below. Furthermore, unless explicitly excluded, antibodies include monoclonal antibodies, bispecific antibodies, minibodies, domain antibodies, synthetic antibodies (sometimes referred to herein as “antibody mimetics”), chimeric antibodies, humanized antibodies, human antibodies, antibody fusions (sometimes referred to herein as “antibody conjugates”), and fragments thereof, respectively. In some embodiments, the term also encompasses peptibodies.

Naturally occurring antibody structural units typically comprise a tetramer. Each such tetramer typically is composed of two identical pairs of polypeptide chains, each pair having one full-length “light” (in certain embodiments, about 25 kDa) and one full-length “heavy” chain (in certain embodiments, about 50-70 kDa). The amino-terminal portion of each chain typically includes a variable region of about 100 to 110 or more amino acids that typically is responsible for antigen recognition. The carboxy-terminal portion of each chain typically defines a constant region that can be responsible for effector function. Human light chains are typically classified as kappa and lambda light chains. Heavy chains are typically classified as mu, delta, gamma, alpha, or epsilon, and define the antibody's isotype as IgM, IgD, IgG, IgA, and IgE, respectively. IgG has several subclasses, including, but not limited to, IgG1, IgG2, IgG3, and IgG4. IgM has subclasses including, but not limited to, IgM1 and IgM2. IgA is similarly subdivided into subclasses including, but not limited to, IgA1 and IgA2. Within full-length light and heavy chains, typically, the variable and constant regions are joined by a “J” region of about 12 or more amino acids, with the heavy chain also including a “D” region of about 10 more amino acids. See, e.g., Fundamental Immunology, Ch. 7 (Paul, W., ed., 2nd ed. Raven Press, N.Y. (1989)) (incorporated by reference in its entirety for all purposes). The variable regions of each light/heavy chain pair typically form the antigen binding site.

The term “variable region” or “variable domain” refers to a portion of the light and/or heavy chains of an antibody, typically including approximately the amino-terminal 120 to 130 amino acids in the heavy chain and about 100 to 110 amino terminal amino acids in the light chain. In certain embodiments, variable regions of different antibodies differ extensively in amino acid sequence even among antibodies of the same species. The variable region of an antibody typically determines specificity of a particular antibody for its target.

The variable regions typically exhibit the same general structure of relatively conserved framework regions (FR) joined by three hyper variable regions, also called complementarity determining regions or CDRs. The CDRs from the two chains of each pair typically are aligned by the framework regions, which can enable binding to a specific epitope. From N-terminal to C-terminal, both light and heavy chain variable regions typically comprise the domains FR1, CDR1, FR2, CDR2, FR3, CDR3 and FR4. The assignment of amino acids to each domain is typically in accordance with the definitions of Kabat Sequences of Proteins of Immunological Interest (National Institutes of Health, Bethesda, Md. (1987 and 1991)), Chothia & Lesk, J. Mol. Biol., 196:901-917 (1987) or Chothia et al., Nature, 342:878-883 (1989).

In certain embodiments, an antibody heavy chain binds to an antigen in the absence of an antibody light chain. In certain embodiments, an antibody light chain binds to an antigen in the absence of an antibody heavy chain. In certain embodiments, an antibody binding region binds to an antigen in the absence of an antibody light chain. In certain embodiments, an antibody binding region binds to an antigen in the absence of an antibody heavy chain. In certain embodiments, an individual variable region specifically binds to an antigen in the absence of other variable regions.

In certain embodiments, definitive delineation of a CDR and identification of residues comprising the binding site of an antibody is accomplished by solving the structure of the antibody and/or solving the structure of the antibody-ligand complex. In certain embodiments, that can be accomplished by any of a variety of techniques known to those skilled in the art, such as X-ray crystallography. In certain embodiments, various methods of analysis can be employed to identify or approximate the CDR regions. Examples of such methods include, but are not limited to, the Kabat definition, the Chothia definition, the AbM definition and the contact definition.

The Kabat definition is a standard for numbering the residues in an antibody and is typically used to identify CDR regions. See, e.g., Johnson & Wu, Nucleic Acids Res., 28: 214-8 (2000). The Chothia definition is similar to the Kabat definition, but the Chothia definition takes into account positions of certain structural loop regions. See, e.g., Chothia et al., J. Mol. Biol., 196: 901-17 (1986); Chothia et al., Nature, 342: 877-83 (1989). The AbM definition uses an integrated suite of computer programs produced by Oxford Molecular Group that model antibody structure. See, e.g., Martin et al., Proc Natl Acad Sci (USA), 86:9268-9272 (1989); “AbM™, A Computer Program for Modeling Variable Regions of Antibodies,” Oxford, UK; Oxford Molecular, Ltd. The AbM definition models the tertiary structure of an antibody from primary sequence using a combination of knowledge databases and ab initio methods, such as those described by Samudrala et al., “Ab Initio Protein Structure Prediction Using a Combined Hierarchical Approach,” in PROTEINS, Structure, Function and Genetics Suppl., 3:194-198 (1999). The contact definition is based on an analysis of the available complex crystal structures. See, e.g., MacCallum et al., J. Mol. Biol., 5:732-45 (1996).

By convention, the CDR regions in the heavy chain are typically referred to as H1, H2, and H3 and are numbered sequentially in the direction from the amino terminus to the carboxy terminus. The CDR regions in the light chain are typically referred to as L1, L2, and L3 and are numbered sequentially in the direction from the amino terminus to the carboxy terminus.

The term “light chain” includes a full-length light chain and fragments thereof having sufficient variable region sequence to confer binding specificity. A full-length light chain includes a variable region domain, VL, and a constant region domain, CL. The variable region domain of the light chain is at the amino-terminus of the polypeptide. Light chains include kappa chains and lambda chains.

The term “heavy chain” includes a full-length heavy chain and fragments thereof having sufficient variable region sequence to confer binding specificity. A full-length heavy chain includes a variable region domain, VH, and three constant region domains, CH1, CH2, and CH3. The VH domain is at the amino-terminus of the polypeptide, and the CH domains are at the carboxyl-terminus, with the CH3 being closest to the carboxy-terminus of the polypeptide. Heavy chains can be of any isotype, including IgG (including IgG1, IgG2, IgG3 and IgG4 subtypes), IgA (including IgAi and IgA2 subtypes), IgM and IgE.

A bispecific or bifunctional antibody typically is an artificial hybrid antibody having two different heavy/light chain pairs and two different binding sites. Bispecific antibodies can be produced by a variety of methods including, but not limited to, fusion of hybridomas or linking of Fab′ fragments. See, e.g., Songsivilai et al., Clin. Exp. Immunol., 79: 315-321 (1990); Kostelny et al., J. Immunol., 148:1547-1553 (1992).

The term “antigen” refers to a substance capable of inducing adaptive immune responses. Specifically, an antigen is a substance which serves as a target for the receptors of an adaptive immune response. Typically, an antigen is a molecule that binds to antigen-specific receptors but cannot induce an immune response in the body by itsself. Antigens are usually proteins and polysaccharides, less frequently also lipids. As used herein, antigens also include immunogens and haptens.

An “antigen binding protein” (“ABP”) as used herein means any protein that binds a specified target antigen. In the instant application, the specified target antigen is the CHI3L1 protein or fragment thereof. “Antigen binding protein” includes but is not limited to antibodies and antigen-binding fragment thereof. Peptibodies are another example of antigen binding proteins.

The term “antigen-binding fragment” as used herein refers to a portion of a protein which is capable of binding specifically to an antigen. In certain embodiment, the antigen-binding fragment is derived from an antibody comprising one or more CDRs, or any other antibody fragment that binds to an antigen but does not comprise an intact native antibody structure. In certain embodiments, the antigen-binding fragment is not derived from an antibody but rather is derived from a receptor. Examples of antigen-binding fragment include, without limitation, a diabody, a Fab, a Fab′, a F(ab′)₂, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a multispecific antibody, a single domain antibody (sdAb), a camelid antibody or a nanobody, a domain antibody, and a bivalent domain antibody. In certain embodiments, an antigen-binding fragment is capable of binding to the same antigen to which the parent antibody binds. In certain embodiments, an antigen-binding fragment may comprise one or more CDRs from a particular human antibody grafted to a framework region from one or more different human antibodies. In certain embodiments, the antigen-binding fragment is derived from a receptor and contains one or more mutations. In certain embodiments, the antigen-binding fragment does not bind to the natural ligand of the receptor from which the antigen-binding fragment is derived.

A “Fab fragment” comprises one light chain and the CH1 and variable regions of one heavy chain. The heavy chain of a Fab molecule cannot form a disulfide bond with another heavy chain molecule.

A “Fab′ fragment” comprises one light chain and a portion of one heavy chain that contains the VH domain and the CH1 domain and also the region between the CH1 and CH2 domains, such that an interchain disulfide bond can be formed between the two heavy chains of two Fab′ fragments to form an F(ab′)₂ molecule.

A “F(ab′)₂ fragment” contains two light chains and two heavy chains containing a portion of the constant region between the CH1 and CH2 domains, such that an interchain disulfide bond is formed between the two heavy chains. A F(ab′)₂ fragment thus is composed of two Fab′ fragments that are held together by a disulfide bond between the two heavy chains.

An “Fc” region comprises two heavy chain fragments comprising the CH1 and CH2 domains of an antibody. The two heavy chain fragments are held together by two or more disulfide bonds and by hydrophobic interactions of the CH3 domains.

The “Fv region” comprises the variable regions from both the heavy and light chains but lacks the constant regions.

“Single-chain antibodies” are Fv molecules in which the heavy and light chain variable regions have been connected by a flexible linker to form a single polypeptide chain, which forms an antigen binding region. Single chain antibodies are discussed in detail in International Patent Application Publication No. WO 88/01649 and U.S. Pat. Nos. 4,946,778 and 5,260,203, the disclosures of which are incorporated by reference.

A “domain antibody” is an immunologically functional immunoglobulin fragment containing only the variable region of a heavy chain or the variable region of a light chain. In some instances, two or more VH regions are covalently joined with a peptide linker to create a bivalent domain antibody. The two VH regions of a bivalent domain antibody can target the same or different antigens.

A “bivalent antigen binding protein” or “bivalent antibody” comprises two antigen binding sites. In some instances, the two binding sites have the same antigen specificities. Bivalent antigen binding proteins and bivalent antibodies can be bispecific, see, infra. A bivalent antibody other than a “multispecific” or “multifunctional” antibody, in certain embodiments, typically is understood to have each of its binding sites identical.

A “multispecific antigen binding protein” or “multispecific antibody” is one that targets more than one antigen or epitope.

A “bispecific,” “dual-specific” or “bifunctional” antigen binding protein or antibody is a hybrid antigen binding protein or antibody, respectively, having two different antigen binding sites. Bispecific antigen binding proteins and antibodies are a species of multispecific antigen binding protein antibody and can be produced by a variety of methods including, but not limited to, fusion of hybridomas or linking of Fab′ fragments. See, e.g., Songsivilai and Lachmann, 1990, Clin. Exp. Immunol. 79:315-321; Kostelny et al., 1992, J. Immunol. 148:1547-1553. The two binding sites of a bispecific antigen binding protein or antibody will bind to two different epitopes, which can reside on the same or different protein targets.

“Binding affinity” generally refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity that reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention. Specific illustrative and exemplary embodiments for measuring binding affinity are described in the following.

An antibody that “specifically binds to” or is “specific for” a particular polypeptide or an epitope on a particular polypeptide is one that binds to that particular polypeptide or epitope on a particular polypeptide without substantially binding to any other polypeptide or polypeptide epitope. For example, the CHI3L1 specific antibodies of the present invention are specific to CHI3L1. In some embodiments, the antibody that binds to CHI3L1 has a dissociation constant (Kd) of ≤100 nM, ≤10 nM, ≤1 nM, ≤0.1 nM, ≤0.01 nM, or ≤0.001 nM (e.g., 10⁻⁸M or less, e.g., from 10⁻⁸M to 10⁻¹³M, e.g., from 10⁻⁹M to 10⁻¹³ M).

The term “compete” when used in the context of antigen binding proteins (e.g., antibody or antigen-binding fragment thereof) that compete for the same epitope means competition between antigen binding proteins as determined by an assay in which the antigen binding protein (e.g., antibody or antigen-binding fragment thereof) being tested prevents or inhibits (e.g., reduces) specific binding of a reference antigen binding protein (e.g., a ligand, or a reference antibody) to a common antigen (e.g., CHI3L1 or a fragment thereof). Numerous types of competitive binding assays can be used to determine if one antigen binding protein competes with another, for example: solid phase direct or indirect radioimmunoassay (RIA), solid phase direct or indirect enzyme immunoassay (EIA), sandwich competition assay (see, e.g., Stahli et al., 1983, Methods in Enzymology 9:242-253); solid phase direct biotin-avidin EIA (see, e.g., Kirkland et al., 1986, J. Immunol. 137:3614-3619) solid phase direct labeled assay, solid phase direct labeled sandwich assay (see, e.g., Harlow and Lane, 1988, Antibodies, A Laboratory Manual, Cold Spring Harbor Press); solid phase direct label RIA using 1-125 label (see, e.g., Morel et al., 1988, Molec. Immunol. 25:7-15); solid phase direct biotin-avidin EIA (see, e.g., Cheung, et al., 1990, Virology 176:546-552); and direct labeled RIA (Moldenhauer et al., 1990, Scand. J. Immunol. 32:77-82). Typically, such an assay involves the use of purified antigen bound to a solid surface or cells bearing either of these, an unlabelled test antigen binding protein and a labeled reference antigen binding protein. Competitive inhibition is measured by determining the amount of label bound to the solid surface or cells in the presence of the test antigen binding protein. Usually the test antigen binding protein is present in excess. Antigen binding proteins identified by competition assay (competing antigen binding proteins) include antigen binding proteins binding to the same epitope as the reference antigen binding proteins and antigen binding proteins binding to an adjacent epitope sufficiently proximal to the epitope bound by the reference antigen binding protein for steric hindrance to occur. Additional details regarding methods for determining competitive binding are provided in the examples herein. Usually, when a competing antigen binding protein is present in excess, it will inhibit (e.g., reduce) specific binding of a reference antigen binding protein to a common antigen by at least 40-45%, 45-50%, 50-55%, 55-60%, 60-65%, 65-70%, 70-75% or 75% or more. In some instances, binding is inhibited by at least 80-85%, 85-90%, 90-95%, 95-97%, or 97% or more.

The term “epitope” as used herein refers to the specific group of atoms or amino acids on an antigen to which an antibody binds. The epitope can be either linear epitope or a conformational epitope. A linear epitope is formed by a continuous sequence of amino acids from the antigen and interacts with an antibody based on their primary structure. A conformational epitope, on the other hand, is composed of discontinuous sections of the antigen's amino acid sequence and interacts with the antibody based on the 3D structure of the antigen. In general, an epitope is approximately five or six amino acid in length. Two antibodies may bind the same epitope within an antigen if they exhibit competitive binding for the antigen.

A “cell”, as used herein, can be prokaryotic or eukaryotic. A prokaryotic cell includes, for example, bacteria. A eukaryotic cell includes, for example, a fungus, a plant cell, and an animal cell. The types of an animal cell (e.g., a mammalian cell or a human cell) includes, for example, a cell from circulatory/immune system or organ, e.g., a B cell, a T cell (cytotoxic T cell, natural killer T cell, regulatory T cell, T helper cell), a natural killer cell, a granulocyte (e.g., basophil granulocyte, an eosinophil granulocyte, a neutrophil granulocyte and a hypersegmented neutrophil), a monocyte or macrophage, a red blood cell (e.g., reticulocyte), a mast cell, a thrombocyte or megakaryocyte, and a dendritic cell; a cell from an endocrine system or organ, e.g., a thyroid cell (e.g., thyroid epithelial cell, parafollicular cell), a parathyroid cell (e.g., parathyroid chief cell, oxyphil cell), an adrenal cell (e.g., chromaffin cell), and a pineal cell (e.g., pinealocyte); a cell from a nervous system or organ, e.g., a glioblast (e.g., astrocyte and oligodendrocyte), a microglia, a magnocellular neurosecretory cell, a stellate cell, a boettcher cell, and a pituitary cell (e.g., gonadotrope, corticotrope, thyrotrope, somatotrope, and lactotroph); a cell from a respiratory system or organ, e.g., a pneumocyte (a type I pneumocyte and a type II pneumocyte), a clara cell, a goblet cell, and an alveolar macrophage; a cell from circular system or organ (e.g., myocardiocyte and pericyte); a cell from digestive system or organ, e.g., a gastric chief cell, a parietal cell, a goblet cell, a paneth cell, a G cell, a D cell, an ECL cell, an I cell, a K cell, an S cell, an enteroendocrine cell, an enterochromaffin cell, an APUD cell, and a liver cell (e.g., a hepatocyte and Kupffer cell); a cell from integumentary system or organ, e.g., a bone cell (e.g., an osteoblast, an osteocyte, and an osteoclast), a teeth cell (e.g., a cementoblast, and an ameloblast), a cartilage cell (e.g., a chondroblast and a chondrocyte), a skin/hair cell (e.g., a trichocyte, a keratinocyte, and a melanocyte (Nevus cell), a muscle cell (e.g., myocyte), an adipocyte, a fibroblast, and a tendon cell; a cell from urinary system or organ (e.g., a podocyte, a juxtaglomerular cell, an intraglomerular mesangial cell, an extraglomerular mesangial cell, a kidney proximal tubule brush border cell, and a macula densa cell); and a cell from reproductive system or organ (e.g., a spermatozoon, a Sertoli cell, a leydig cell, an ovum, an oocyte). A cell can be normal, healthy cell; or a diseased or unhealthy cell (e.g., a cancer cell). A cell further includes a mammalian zygote or a stem cell which include an embryonic stem cell, a fetal stem cell, an induced pluripotent stem cell, and an adult stem cell. A stem cell is a cell that is capable of undergoing cycles of cell division while maintaining an undifferentiated state and differentiating into specialized cell types. A stem cell can be an omnipotent stem cell, a pluripotent stem cell, a multipotent stem cell, an oligopotent stem cell and a unipotent stem cell, any of which may be induced from a somatic cell. A stem cell may also include a cancer stem cell. A mammalian cell can be a rodent cell, e.g., a mouse, rat, hamster cell. A mammalian cell can be a lagomorpha cell, e.g., a rabbit cell. A mammalian cell can also be a primate cell, e.g., a human cell.

The term “chimeric antigen receptor” or “CAR” as used herein refers to an artificially constructed hybrid protein or polypeptide containing an antigen binding domain of an antibody (e.g., a single chain variable fragment (scFv)) linked to a domain or signaling, e.g., T-cell signaling or T-cell activation domains, that activates an immune cell, e.g., a T cell or a NK cell (see, e.g., Kershaw et al., supra, Eshhar et al., Proc. Natl. Acad. Sci. USA, 90(2): 720-724 (1993), and Sadelain et al., Curr. Opin. Immunol. 21(2): 215-223 (2009)). CARs are capable of redirecting the immune cell specificity and reactivity toward a selected target in a non-MHC-restricted manner, taking advantage of the antigen-binding properties of monoclonal antibodies. The non-MHC-restricted antigen recognition confers immune cells expressing CARs on the ability to recognize an antigen independent of antigen processing, thus bypassing a major mechanism of tumor escape. In addition, when expressed in T-cells, CARs advantageously do not dimerize with endogenous T-cell receptor (TCR) alpha and beta chains.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

The term “host cell” means a cell that has been transformed, or is capable of being transformed, with a nucleic acid sequence and thereby expresses a gene of interest. The term includes the progeny of the parent cell, whether or not the progeny is identical in morphology or in genetic make-up to the original parent cell, so long as the gene of interest is present.

The term “identity” refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) are preferably addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al., 1988, SIAM J. Applied Math. 48:1073.

In calculating percent identity, the sequences being compared are typically aligned in a way that gives the largest match between the sequences. One example of a computer program that can be used to determine percent identity is the GCG program package, which includes GAP (Devereux et al., 1984, Nucl. Acid Res. 12:387; Genetics Computer Group, University of Wisconsin, Madison, Wis.). The computer algorithm GAP is used to align the two polypeptides or polynucleotides for which the percent sequence identity is to be determined. The sequences are aligned for optimal matching of their respective amino acid or nucleotide (the “matched span”, as determined by the algorithm). A gap opening penalty (which is calculated as 3× the average diagonal, wherein the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. In certain embodiments, a standard comparison matrix (see, Dayhoff et al., 1978, Atlas of Protein Sequence and Structure 5:345-352 for the PAM 250 comparison matrix; Henikoff et al., 1992, Proc. Natl. Acad. Sci. U.S.A. 89:10915-10919 for the BLOSUM 62 comparison matrix) is also used by the algorithm.

Examples of parameters that can be employed in determining percent identity for polypeptides or nucleotide sequences using the GAP program can be found in Needleman et al., 1970, J. Mol. Biol. 48:443-453.

Certain alignment schemes for aligning two amino acid sequences may result in matching of only a short region of the two sequences, and this small aligned region may have very high sequence identity even though there is no significant relationship between the two full-length sequences. Accordingly, the selected alignment method (GAP program) can be adjusted if so desired to result in an alignment that spans at least 50 or other number of contiguous amino acids of the target polypeptide.

The term “link” as used herein refers to the association via intramolecular interaction, e.g., covalent bonds, metallic bonds, and/or ionic bonding, or inter-molecular interaction, e.g., hydrogen bond or noncovalent bonds.

The term “operably linked” refers to an arrangement of elements wherein the components so described are configured so as to perform their usual function. Thus, a given signal peptide that is operably linked to a polypeptide directs the secretion of the polypeptide from a cell. In the case of a promoter, a promoter that is operably linked to a coding sequence will direct the expression of the coding sequence. The promoter or other control elements need not be contiguous with the coding sequence, so long as they function to direct the expression thereof. For example, intervening untranslated yet transcribed sequences can be present between the promoter sequence and the coding sequence and the promoter sequence can still be considered “operably linked” to the coding sequence.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

The term “polynucleotide” or “nucleic acid” includes both single-stranded and double-stranded nucleotide polymers. The nucleotides comprising the polynucleotide can be ribonucleotides or deoxyribonucleotides or a modified form of either type of nucleotide. Said modifications include base modifications such as bromouridine and inosine derivatives, ribose modifications such as 2′,3′-dideoxyribose, and internucleotide linkage modifications such as phosphorothioate, phosphorodithioate, phosphoroselenoate, phosphorodiselenoate, phosphoroanilothioate, phoshoraniladate and phosphoroamidate.

The terms “polypeptide” or “protein” means a macromolecule having the amino acid sequence of a native protein, that is, a protein produced by a naturally-occurring and non-recombinant cell; or it is produced by a genetically-engineered or recombinant cell, and comprise molecules having the amino acid sequence of the native protein, or molecules having deletions from, additions to, and/or substitutions of one or more amino acids of the native sequence. The term also includes amino acid polymers in which one or more amino acids are chemical analogs of a corresponding naturally-occurring amino acid and polymers. The terms “polypeptide” and “protein” specifically encompass CHI3L1 binding proteins, antibodies, or sequences that have deletions from, additions to, and/or substitutions of one or more amino acid of antigen-binding protein. The term “polypeptide fragment” refers to a polypeptide that has an amino-terminal deletion, a carboxyl-terminal deletion, and/or an internal deletion as compared with the full-length native protein. Such fragments can also contain modified amino acids as compared with the native protein. In certain embodiments, fragments are about five to 500 amino acids long. For example, fragments can be at least 5, 6, 8, 10, 14, 20, 50, 70, 100, 110, 150, 200, 250, 300, 350, 400, or 450 amino acids long. Useful polypeptide fragments include immunologically functional fragments of antibodies, including binding domains. In the case of a CHI3L1-binding antibody, useful fragments include but are not limited to a CDR region, a variable domain of a heavy and/or light chain, a portion of an antibody chain or just its variable region including two CDRs, and the like.

The pharmaceutically acceptable carriers useful in this invention are conventional. Remington's Pharmaceutical Sciences, by E. W. Martin, Mack Publishing Co., Easton, PA, 15th Edition (1975), describes compositions and formulations suitable for pharmaceutical delivery of the fusion proteins herein disclosed. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

The term “therapeutically effective amount” or “effective dosage” as used herein refers to the dosage or concentration of a drug effective to treat a disease or condition. For example, with regard to the use of the monoclonal antibodies or antigen-binding fragments thereof disclosed herein to treat cancer, a therapeutically effective amount is the dosage or concentration of the monoclonal antibody or antigen-binding fragment thereof capable of reducing the tumor volume, eradicating all or part of a tumor, inhibiting or slowing tumor growth or cancer cell infiltration into other organs, inhibiting growth or proliferation of cells mediating a cancerous condition, inhibiting or slowing tumor cell metastasis, ameliorating any symptom or marker associated with a tumor or cancerous condition, preventing or delaying the development of a tumor or cancerous condition, or some combination thereof.

“Treating” or “treatment” of a condition as used herein includes preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof.

As used herein, a “vector” refers to a nucleic acid molecule as introduced into a host cell, thereby producing a transformed host cell. A vector may include nucleic acid sequences that permit it to replicate in the host cell, such as an origin of replication. A vector may also include one or more therapeutic genes and/or selectable marker genes and other genetic elements known in the art. A vector can transduce, transform or infect a cell, thereby causing the cell to express nucleic acids and/or proteins other than those native to the cell. A vector optionally includes materials to aid in achieving entry of the nucleic acid into the cell, such as a viral particle, liposome, protein coating or the like.

### II. Chitinase 3-Like-1 and Disease States

### A. Chitinase 3-Like-1

Chitinase-3-like protein 1 (CHI3L1), also known as YKL-40, is a secreted glycoprotein that is approximately 40 kDa in size (human protein at NP_001267) that in humans is encoded by the CHI3L1 gene (human mRNA at NM_001276). The name YKL-40 is derived from the three N-terminal amino acids present on the secreted form and its molecular mass. YKL-40 is expressed and secreted by various cell-types including macrophages, chondrocytes, fibroblast-like synovial cells, vascular smooth muscle cells, and hepatic stellate cells. The biological function of YKL-40 is unclear. It is not known to have a specific receptor. Its pattern of expression is associated with pathogenic processes related to inflammation, extracellular tissue remodeling, fibrosis and solid carcinomas and asthma.

Chitinases catalyze the hydrolysis of chitin, which is an abundant glycopolymer found in insect exoskeletons and fungal cell walls. The glycoside hydrolase 18 family of chitinases includes eight human family members. This gene encodes a glycoprotein member of the glycosyl hydrolase 18 family. The protein lacks chitinase activity and is secreted by activated macrophages, chondrocytes, neutrophils and synovial cells. The protein is thought to play a role in the process of inflammation and tissue remodeling. YKL-40 lacks chitinase activity due to mutations within the active site (conserved sequence: DXXDXDXE (SEQ ID NO: 141); YKL-40 sequence: DGLDLAWL (SEQ ID NO: 142)).

YKL-40 has been linked to activation of the AKT pro-survival (anti-apoptotic) signaling pathway. YKL-40 promotes angiogenesis through VEGF-dependent and independent pathways. YKL-40 is a migration factor for primary astrocytes and its expression is controlled by NFI-X3, STAT3, and AP-1. CHI3L1 is induced by a variety of cancers and in the presence of semaphorin 7A (protein) can inhibit multiple anti-tumor immune system responses. Activating an antiviral immune pathway known as the RIG-like helicase (RLH) has the ability to counter CHI3L1 induction. Cancer cells can offset RLH by stimulating NLRX1. Poly(I:C), an RNA-like molecule, can stimulate RLH activation. RLH activation can also inhibit the expression of receptor IL-13Ru2p. It stores NK cell accumulation and activation. It augments the expression of IFN-α/p, chemerin and its receptor ChemR23, p-cofilin, LIMK2 and PTEN and inhibiting BRAF and NLRX1 in a MAVS-dependent manner.

It is assumed that YKL-40 plays a role in cancer cell proliferation, survival, invasiveness and in the regulation of cell-matrix interactions. It is suggested that YKL-40 is a marker associated with a poorer clinical outcome in genetically defined subgroups of different tumors. YKL-40 was recently introduced into (restricted) clinical practice. A few techniques are available for its detection.

YKL-40 is a Th2 promoting cytokine that is present at high levels in the tumor microenvironment and in the serum of cancer patients. Elevated levels of YKL-40 correlate strongly with stage and outcome of various types of cancer, which establish YKL-40 as a biomarker of disease severity. Targeting YKL-40 with neutralizing antibodies is effective as a treatment in animal models of glioblastoma multiforme. YKL-40 also enhances tumor survival in response to gamma-irradiation.

### B. Cancers

While hyperproliferative diseases can be associated with any disease which causes a cell to begin to reproduce uncontrollably, the prototypical example is cancer. One of the key elements of cancer is that the cell's normal apoptotic cycle is interrupted and thus agents that interrupt the growth of the cells are important as therapeutic agents for treating these diseases. In this disclosure, the tubulysin analogs described herein may be used to lead to decreased cell counts and as such can potentially be used to treat a variety of types of cancer lines. In some aspects, it is anticipated that the tubulysin analogs described herein may be used to treat virtually any malignancy. Here, the only requirement is the presence of CHI3L1 on the surface of the cancer cell, and in particular on the surface of cancer stem cells.

Cancer cells that may be treated according to the present disclosure include but are not limited to cells from the bladder, blood, bone, bone marrow, brain, breast, colon, esophagus, gastrointestinal, gum, head, kidney, liver, lung, nasopharynx, neck, ovary, prostate, skin, stomach, pancreas, testis, tongue, cervix, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; non-encapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; Mullerian mixed tumor; nephroblastoma; hepatoblastoma; carcinosarcoma; mesenchymoma, malignant; Brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In certain aspects, the tumor may comprise an osteosarcoma, angiosarcoma, rhabdosarcoma, leiomyosarcoma, Ewing sarcoma, glioblastoma, neuroblastoma, or leukemia.

### C. Liver Injury

Liver injury, also known as hepatotoxicity, implies chemical-driven liver damage. Drug-induced liver injury is a cause of acute and chronic liver disease. The liver plays a central role in transforming and clearing chemicals and is susceptible to the toxicity from these agents. Certain medicinal agents, when taken in overdoses and sometimes even when introduced within therapeutic ranges, may injure the organ. Other chemical agents, such as those used in laboratories and industries, natural chemicals (e.g., microcystins) and herbal remedies can also induce hepatotoxicity. Chemicals that cause liver injury are called hepatotoxins.

More than 900 drugs have been implicated in causing liver injury and it is the most common reason for a drug to be withdrawn from the market. Hepatotoxicity and drug-induced liver injury also account for a substantial number of compound failures, highlighting the need for toxicity prediction models (e.g., DTI), and drug screening assays, such as stem cell-derived hepatocyte-like cells, that are capable of detecting toxicity early in the drug development process. Chemicals often cause subclinical injury to the liver, which manifests only as abnormal liver enzyme tests. Drug-induced liver injury is responsible for 5% of all hospital admissions and 50% of all acute liver failures.

Adverse drug reactions are classified as type A (intrinsic or pharmacological) or type B (idiosyncratic). Type A drug reaction accounts for 80% of all toxicities. Drugs or toxins that have a pharmacological (type A) hepatotoxicity are those that have predictable dose-response curves (higher concentrations cause more liver damage) and well characterized mechanisms of toxicity, such as directly damaging liver tissue or blocking a metabolic process. As in the case of acetaminophen overdose, this type of injury occurs shortly after some threshold for toxicity is reached.

Idiosyncratic (type B) injury occurs without warning, when agents cause non-predictable hepatotoxicity in susceptible individuals, which is not related to dose and has a variable latency period. This type of injury does not have a clear dose-response nor temporal relationship, and most often does not have predictive models. Idiosyncratic hepatotoxicity has led to the withdrawal of several drugs from market even after rigorous clinical testing as part of the FDA approval process; Troglitazone (Rezulin) and trovafloxacin (Trovan) are two prime examples of idiosyncratic hepatotoxins pulled from market. Oral use of ketoconazole has been associated with hepatic toxicity, including some fatalities; however, such effects appear to be limited to doses taken over a period longer than 7 days.

Acetaminophen (paracemtaol) overdose is the most common cause of drug-induced liver disease. Acetaminophen (in the U.S. and Japan), paracetamol (INN), also known by the brand name Tylenol and Panadol, is usually well tolerated in prescribed dose, but overdose is the most common cause of drug-induced liver disease and acute liver failure worldwide. Damage to the liver is not due to the drug itself but to a toxic metabolite (N-acetyl-p-benzoquinone imine (NAPQI)) produced by cytochrome P-450 enzymes in the liver. In normal circumstances, this metabolite is detoxified by conjugating with glutathione in phase 2 reaction. In an overdose, a large amount of NAPQI is generated, which overwhelms the detoxification process and leads to liver cell damage. Nitric oxide also plays a role in inducing toxicity. The risk of liver injury is influenced by several factors including the dose ingested, concurrent alcohol or other drug intake, interval between ingestion and antidote, etc. The dose toxic to the liver is quite variable from person to person and is often thought to be lower in chronic alcoholics. Measurement of blood level is important in assessing prognosis, higher levels predicting a worse prognosis. Administration of Acetylcysteine, a precursor of glutathione, can limit the severity of the liver damage by capturing the toxic NAPQI. Those that develop acute liver failure can still recover spontaneously, but may require transplantation if poor prognostic signs such as encephalopathy or coagulopathy is present (see King's College Criteria).

Nonsteroidal anti-inflammatory drugs. Although individual analgesics rarely induce liver damage due to their widespread use, NSAIDs have emerged as a major group of drugs exhibiting hepatotoxicity. Both dose-dependent and idiosyncratic reactions have been documented. Aspirin and phenylbutazone are associated with intrinsic hepatotoxicity; idiosyncratic reaction has been associated with ibuprofen, sulindac, phenylbutazone, piroxicam, diclofenac and indomethacin.

Glucocorticoids. Glucocorticoids are so named due to their effect on the carbohydrate mechanism. They promote glycogen storage in the liver. An enlarged liver is a rare side-effect of long-term steroid use in children. The classical effect of prolonged use both in adult and pediatric population is steatosis.

Isoniazid. Isoniazide (INH) is one of the most commonly used drugs for tuberculosis; it is associated with mild elevation of liver enzymes in up to 20% of patients and severe hepatotoxicity in 1-2% of patients.

Other hydrazine derivative drugs. There are also cases where other hydrazine derivative drugs, such as the MAOI antidepressant iproniazid, are associated with liver damage. Phenelzine has been associated with abnormal liver tests. Toxic effects can develop from antibiotics.

Natural products. Examples include many amanita mushrooms (particularly the destroying angels), and aflatoxins. Pyrrolizidine alkaloids, which occur in some plants, can be toxic. Green tea extract is a growing cause of liver failure due to its inclusion in more products.

Industrial toxins. Examples include arsenic, carbon tetrachloride, and vinyl chloride.

Alternative remedies. Examples include Ackee fruit, Bajiaolian, Camphor, Copaltra, Cycasin, Garcinia, Kava leaves, pyrrolizidine alkaloids, Horse chestnut leaves, Valerian, Comfrey. Chinese herbal remedies: Jin Bu Huan, Ma-huang, Shou Wu Pian, Bai Xian Pi.

### III. Monoclonal Antibodies and Production Thereof

The monoclonal antibodies described herein can be prepared using standard methods, followed by screening, characterization and functional assessment. Variable regions can be sequenced and then subcloned into a human expression vector to produce the chimeric antibody genes, which are then expressed and purified. These chimeric antibodies can be tested for antigen binding, signaling blocking, and in xenograft experiments.

### A. General Methods

It will be understood that monoclonal antibodies binding to CHI3L1 will have several applications. These include the production of diagnostic kits for use in detecting and diagnosing cancer, as well as for cancer therapies. In these contexts, one may link such antibodies to diagnostic or therapeutic agents, use them as capture agents or competitors in competitive assays, or use them individually without additional agents being attached thereto. The antibodies may be mutated or modified, as discussed further below. Methods for preparing and characterizing antibodies are well known in the art (see, e.g., Antibodies: A Laboratory Manual, Cold Spring Harbor Laboratory, 1988; U.S. Pat. No. 4,196,265).

The methods for generating monoclonal antibodies (MAbs) generally begin along the same lines as those for preparing polyclonal antibodies. The first step for both these methods is immunization of an appropriate host. As is well known in the art, a given composition for immunization may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimyde and bis-biazotized benzidine. As also is well known in the art, the immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster injection, also may be given. The process of boosting and titering is repeated until a suitable titer is achieved. When a desired level of immunogenicity is obtained, the immunized animal can be bled and the serum isolated and stored, and/or the animal can be used to generate MAbs.

Following immunization, somatic cells with the potential for producing antibodies, specifically B lymphocytes (B cells), are selected for use in the MAb generating protocol. These cells may be obtained from biopsied spleens or lymph nodes, or from circulating blood. The antibody-producing B lymphocytes from the immunized animal are then fused with cells of an immortal myeloma cell, generally one of the same species as the animal that was immunized or human or human/mouse chimeric cells. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas). Any one of a number of myeloma cells may be used, as are known to those of skill in the art (Goding, pp. 65-66, 1986; Campbell, pp. 75-83, 1984).

Methods for generating hybrids of antibody-producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. Fusion methods using Sendai virus have been described by Kohler and Milstein (1975; 1976), and those using polyethylene glycol (PEG), such as 37% (v/v) PEG, by Gefter et al. (1977). The use of electrically induced fusion methods also is appropriate (Goding, pp. 71-74, 1986). Fusion procedures usually produce viable hybrids at low frequencies, about 1×10⁻⁶ to 1×10⁻⁸. However, this does not pose a problem, as the viable, fused hybrids are differentiated from the parental, infused cells (particularly the infused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine. Ouabain is added if the B cell source is an Epstein Barr virus (EBV) transformed human B cell line, in order to eliminate EBV transformed lines that have not fused to the myeloma.

The preferred selection medium is HAT or HAT with ouabain. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells. When the source of B cells used for fusion is a line of EBV-transformed B cells, as here, ouabain is also used for drug selection of hybrids as EBV-transformed B cells are susceptible to drug killing, whereas the myeloma partner used is chosen to be ouabain resistant.

Culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays dot immunobinding assays, and the like. The selected hybridomas are then serially diluted or single-cell sorted by flow cytometric sorting and cloned into individual antibody-producing cell lines, which clones can then be propagated indefinitely to provide mAbs. The cell lines may be exploited for MAb production in two basic ways. A sample of the hybridoma can be injected (often into the peritoneal cavity) into an animal (e.g., a mouse). Optionally, the animals are primed with a hydrocarbon, especially oils such as pristane (tetramethylpentadecane) prior to injection. When human hybridomas are used in this way, it is optimal to inject immunocompromised mice, such as SCID mice, to prevent tumor rejection. The injected animal develops tumors secreting the specific monoclonal antibody produced by the fused cell hybrid. The body fluids of the animal, such as serum or ascites fluid, can then be tapped to provide MAbs in high concentration. The individual cell lines could also be cultured in vitro, where the MAbs are naturally secreted into the culture medium from which they can be readily obtained in high concentrations. Alternatively, human hybridoma cells lines can be used in vitro to produce immunoglobulins in cell supernatant. The cell lines can be adapted for growth in serum-free medium to optimize the ability to recover human monoclonal immunoglobulins of high purity.

MAbs produced by either means may be further purified, if desired, using filtration, centrifugation and various chromatographic methods such as FPLC or affinity chromatography. Fragments of the monoclonal antibodies of the disclosure can be obtained from the purified monoclonal antibodies by methods which include digestion with enzymes, such as pepsin or papain, and/or by cleavage of disulfide bonds by chemical reduction. Alternatively, monoclonal antibody fragments encompassed by the present disclosure can be synthesized using an automated peptide synthesizer.

It also is contemplated that a molecular cloning approach may be used to generate monoclonals. For this, RNA can be isolated from the hybridoma line and the antibody genes obtained by RT-PCR and cloned into an immunoglobulin expression vector. Alternatively, combinatorial immunoglobulin phagemid libraries are prepared from RNA isolated from the cell lines and phagemids expressing appropriate antibodies are selected by panning using viral antigens. The advantages of this approach over conventional hybridoma techniques are that approximately 104 times as many antibodies can be produced and screened in a single round, and that new specificities are generated by H and L chain combination which further increases the chance of finding appropriate antibodies.

Other U.S. patents, each incorporated herein by reference, that teach the production of antibodies useful in the present disclosure include U.S. Pat. No. 5,565,332, which describes the production of chimeric antibodies using a combinatorial approach; U.S. Pat. No. 4,816,567 which describes recombinant immunoglobulin preparations; and U.S. Pat. No. 4,867,973 which describes antibody-therapeutic agent conjugates.

### B. Antibodies of the Present Disclosure

### 1. Antibodies to CHI3L1

Antibodies or antigen-binding fragments thereof according to the present disclosure may be defined, in the first instance, by their binding specificity, which in this case is for CHI3L1. Those of skill in the art, by assessing the binding specificity/affinity of a given antibody using techniques well known to those of skill in the art, can determine whether such antibodies fall within the scope of the instant claims.

In one aspect, there are provided antibodies and antigen-binding fragments that specifically bind to CHI3L1. In some embodiments, when bound to CHI3L1, such antibodies modulate the activation of CHI3L1.

In some embodiments, the antibodies or antigen-binding fragments provided herein having clone-paired CDR's from the heavy chains and light chains illustrated in the tables below. Such antibodies may be produced by the clones discussed below in the Examples section using methods described herein. In certain embodiments, each CDR is defined in accordance with Kabat definition, the Chothia definition, the combination of Kabat definition and Chothia definition, the AbM definition, or the contact definition of CDR. In certain embodiments, the antibody or antigen-binding fragment is characterized by clone-paired heavy and light chain sequences from the tables below.

In certain embodiments, the antibodies may be defined by their variable sequence, which include additional “framework” regions. The antibody is characterized by clone-paired heavy chain and light chain amino acid sequences from the tables below. Furthermore, the antibodies sequences may vary from these sequences, particularly in regions outside the CDRs. For example, the amino acids may vary from those set out above by a given percentage, e.g., 80%, 85%, 90%, 91%, 92%, 93%, 94%, 95%, 96%, 97%, 98% or 99% homology, or the amino acids may vary from those set out above by permitting conservative substitutions (discussed below). Each of the foregoing apply to the amino acid sequences of the tables below. In another embodiment, the antibody derivatives of the present disclosure comprise VL and VH domains having up to 0, 1, 2, 3, 4, 5, 6, 7, 8, 9, 10 or more conservative or non-conservative amino acid substitutions, while still exhibiting the desired binding and functional properties.

While the antibodies of the present disclosure were generated as IgG's, it may be useful to modify the constant regions to alter their function. The constant regions of the antibodies typically mediate the binding of the antibody to host tissues or factors, including various cells of the immune system (e.g., effector cells) and the first component (Clq) of the classical complement system. Thus, the term “antibody” includes intact immunoglobulins of types IgA, IgG, IgE, IgD, IgM (as well as subtypes thereof), wherein the light chains of the immunoglobulin may be of types kappa or lambda. Within light and heavy chains, the variable and constant regions are joined by a 35 “J” region of about 12 or more amino acids, with the heavy chain also including a “D” region of about 10 more amino acids. See generally, Fundamental Immunology Ch. 7 (Paul, W., ed., 2^{nd} ed. Raven Press, N.Y. (1989).

The present disclosure further comprises nucleic acids which hybridize to nucleic acids encoding the antibodies disclosed herein. In general, the nucleic acids hybridize under moderate or high stringency conditions to nucleic acids that encode antibodies disclosed herein and also encode antibodies that maintain the ability to specifically bind to an CHI3L1. A first nucleic acid molecule is “hybridizable” to a second nucleic acid molecule when a single stranded form of the first nucleic acid molecule can anneal to the second nucleic acid molecule under the appropriate conditions of temperature and solution ionic strength (see Sambrook et al., MOLECULAR CLONING: A LABORATORY MANUAL, 3^{rd} ed., Cold Spring Harbor Press, Cold Spring Harbor, N.Y. 2001). The conditions of temperature and ionic strength determine the “stringency” of the hybridization. Typical moderate stringency hybridization conditions are 40% formamide, with 5× or 6×SSC and 0.1% SDS at 42° C. High stringency hybridization conditions are 50% formamide, 5× or 6×SSC (0.15M NaCl and 0.015M Na-citrate) at 42° C. or, optionally, at a higher temperature (e.g., 57° C., 59° C., 60° C., 62° C., 63° C., 65° C. or 68° C.). Hybridization requires that the two nucleic acids contain complementary sequences, although, depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the higher the stringency under which the nucleic acids may hybridize. For hybrids of greater than 100 nucleotides in length, equations for calculating the melting temperature have been derived (see Sambrook et al., supra). For hybridization with shorter nucleic acids, e.g., oligonucleotides, the position of mismatches becomes more important, and the length of the oligonucleotide determines its specificity (see Sambrook et al., supra).

### 2. Exemplary Epitopes and Competing Antigen Binding Proteins

In another aspect, the present disclosure provides epitopes to which anti-CHI3L1 antibodies bind.

In some embodiments, epitopes that are bound by the antibodies described herein are useful. In certain embodiments, an epitope provided herein can be utilized to isolate antibodies or antigen binding proteins that bind to CHI3L1. In certain embodiments, an epitope provided herein can be utilized to generate antibodies or antigen binding proteins which bind to CHI3L1. In certain embodiments, an epitope or a sequence comprising an epitope provided herein can be utilized as an immunogen to generate antibodies or antigen binding proteins that bind to CHI3L1. In certain embodiments, an epitope described herein or a sequence comprising an epitope described herein can be utilized to interfere with biological activity of CHI3L1.

In some embodiments, antibodies or antigen-binding fragments thereof that bind to any of the epitopes are particularly useful. In some embodiments, an epitope provided herein, when bound by an antibody, modulates the biological activity of CHI3L1.

In some embodiments, the domain(s)/region(s) containing residues that are in contact with or are buried by an antibody can be identified by mutating specific residues in CHI3L1 and determining whether the antibody can bind the mutated CHI3L1 protein. By making a number of individual mutations, residues that play a direct role in binding or that are in sufficiently close proximity to the antibody such that a mutation can affect binding between the antibody and antigen can be identified. From knowledge of these amino acids, the domain(s) or region(s) of the antigen that contain residues in contact with the antigen binding protein or covered by the antibody can be elucidated. Such a domain can include the binding epitope of an antigen binding protein.

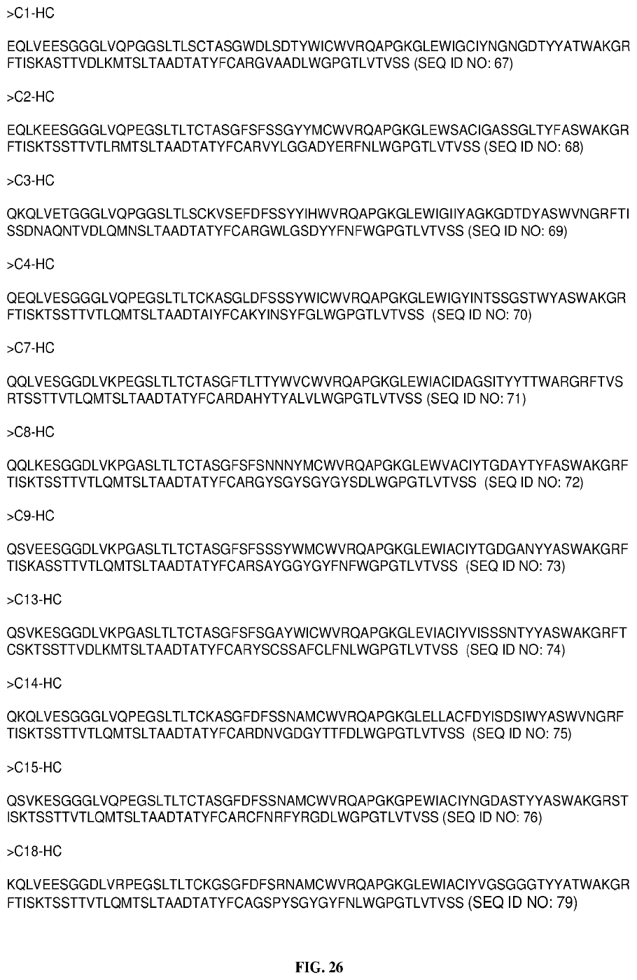

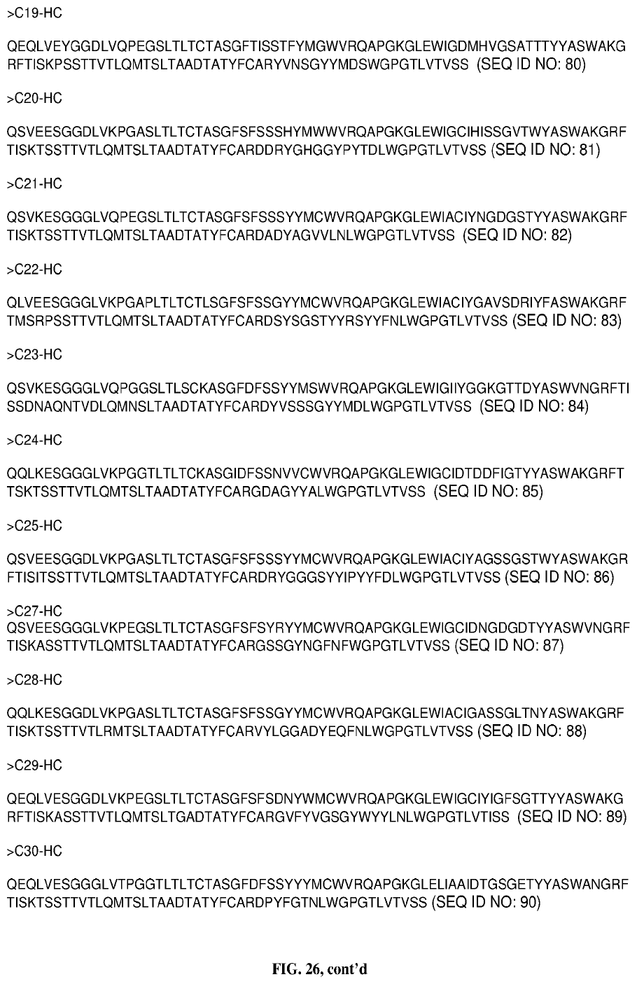

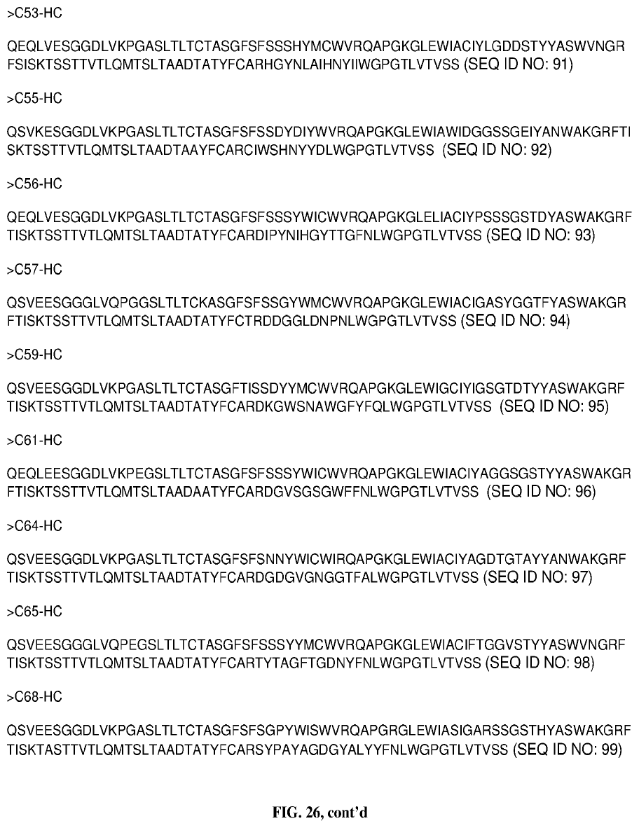

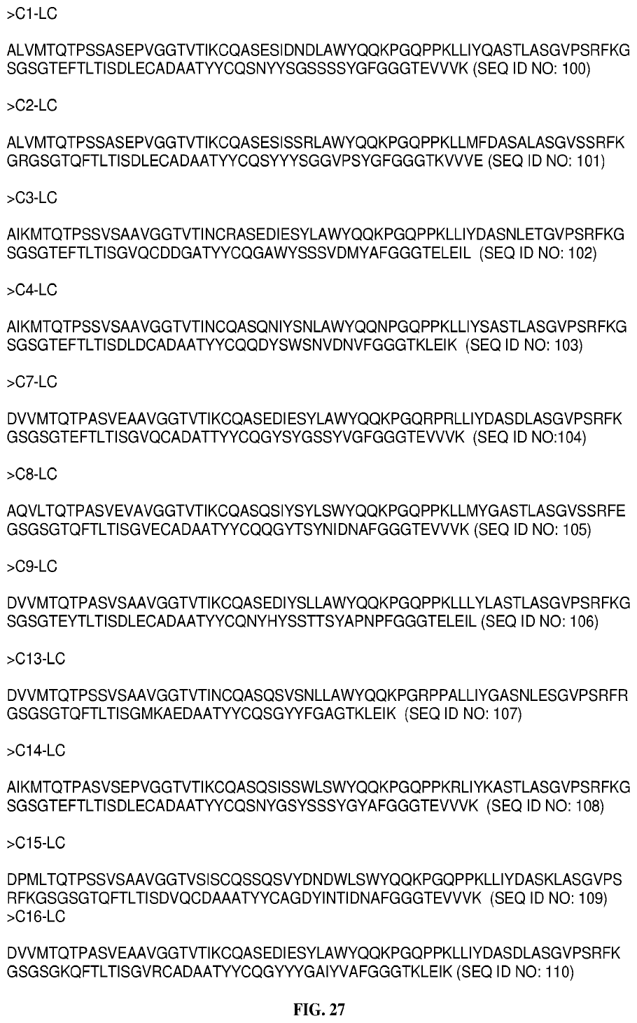

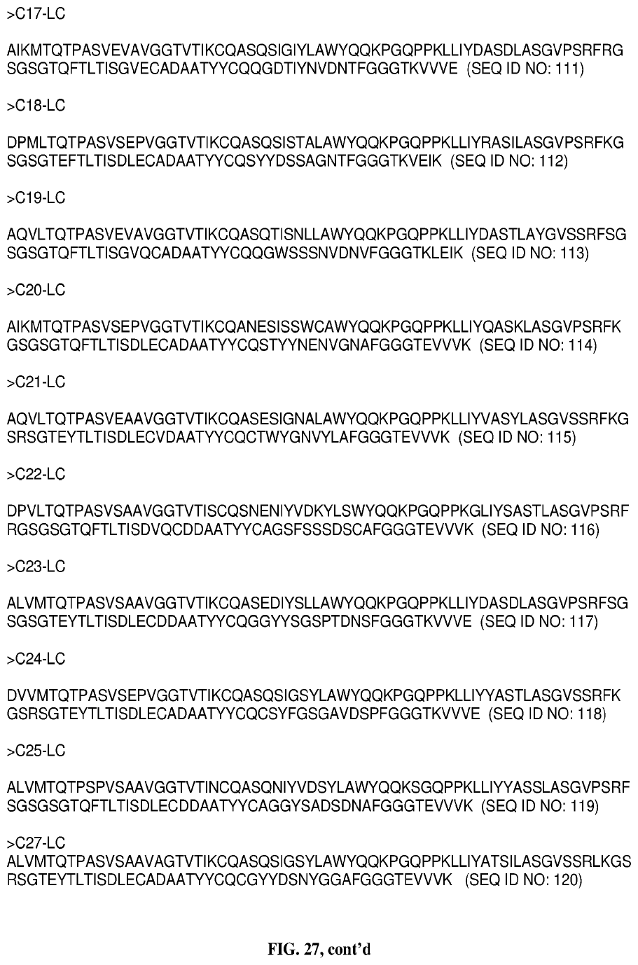

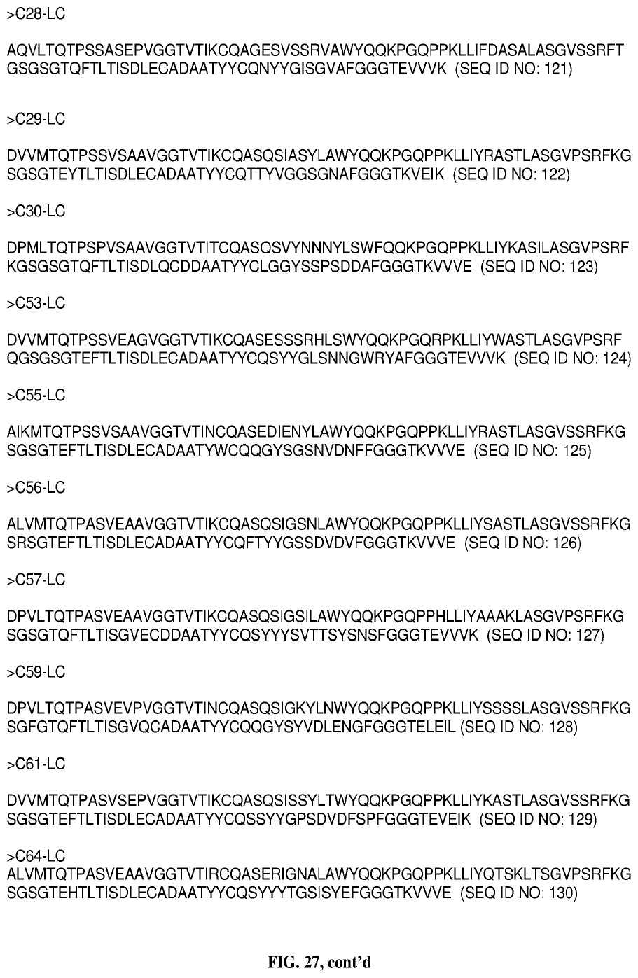

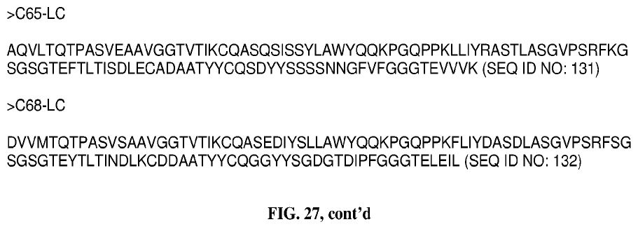

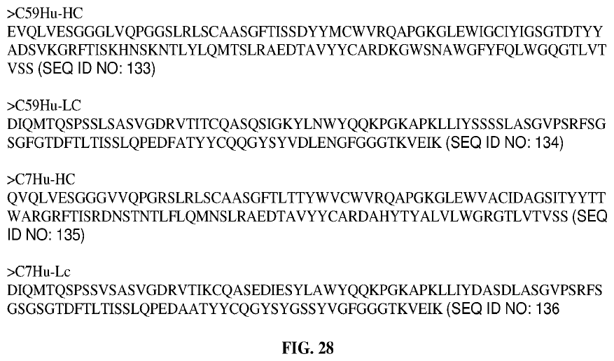

In another aspect, the present disclosure provides antigen-binding proteins that compete with one of the exemplified antibodies or antigen-binding fragment binding to the epitope described herein for specific binding to CHI3L1. Such antigen binding proteins can also bind to the same epitope as one of the herein exemplified antibodies or the antigen-binding fragment, or an overlapping epitope. Antigen-binding proteins that compete with or bind to the same epitope as the exemplified antibodies are expected to show similar functional properties. The exemplified antibodies include those described above, including those with the heavy and light chain variable regions and CDRs included in FIGS. 26, 27 and 28 and Table A.

### C. Engineering of Antibody Sequences

In various embodiments, one may choose to engineer sequences of the identified antibodies for a variety of reasons, such as improved expression, improved cross-reactivity or diminished off-target binding. The following is a general discussion of relevant techniques for antibody engineering.

Hybridomas may be cultured, then cells lysed, and total RNA extracted. Random hexamers may be used with RT to generate cDNA copies of RNA, and then PCR performed using a multiplex mixture of PCR primers expected to amplify all human variable gene sequences. PCR product can be cloned into pGEM-T Easy vector, then sequenced by automated DNA sequencing using standard vector primers. Assay of binding and neutralization may be performed using antibodies collected from hybridoma supernatants and purified by FPLC, using Protein G columns. Recombinant full-length IgG antibodies may be generated by subcloning heavy and light chain Fv DNAs from the cloning vector into an IgG plasmid vector, transfected into 293 Freestyle cells or CHO cells, and antibodies collected a purified from the 293 or CHO cell supernatant.

The rapid availability of antibody produced in the same host cell and cell culture process as the final cGMP manufacturing process has the potential to reduce the duration of process development programs. Lonza has developed a generic method using pooled transfectants grown in CDACF medium, for the rapid production of small quantities (up to 50 g) of antibodies in CHO cells. Although slightly slower than a true transient system, the advantages include a higher product concentration and use of the same host and process as the production cell line. Example of growth and productivity of GS-CHO pools, expressing a model antibody, in a disposable bioreactor: in a disposable bag bioreactor culture (5 L working volume) operated in fed-batch mode, a harvest antibody concentration of 2 g/L was achieved within 9 weeks of transfection.

Antibody molecules will comprise fragments (such as F(ab′), F(ab′)₂) that are produced, for example, by the proteolytic cleavage of the mAbs, or single-chain immunoglobulins producible, for example, via recombinant means. Such antibody derivatives are monovalent. In one embodiment, such fragments can be combined with one another, or with other antibody fragments or receptor ligands to form “chimeric” binding molecules. Significantly, such chimeric molecules may contain substituents capable of binding to different epitopes of the same molecule.

### 1. Antigen Binding Modifications

In related embodiments, the antibody is a derivative of the disclosed antibodies, e.g., an antibody comprising the CDR sequences identical to those in the disclosed antibodies (e.g., a chimeric, or CDR-grafted antibody). Alternatively, one may wish to make modifications, such as introducing conservative changes into an antibody molecule. In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: basic amino acids: arginine (+3.0), lysine (+3.0), and histidine (−0.5); acidic amino acids: aspartate (+3.0±1), glutamate (+3.0±1), asparagine (+0.2), and glutamine (+0.2); hydrophilic, nonionic amino acids: serine (+0.3), asparagine (+0.2), glutamine (+0.2), and threonine (−0.4), sulfur containing amino acids: cysteine (−1.0) and methionine (−1.3); hydrophobic, nonaromatic amino acids: valine (−1.5), leucine (−1.8), isoleucine (−1.8), proline (−0.5±1), alanine (−0.5), and glycine (0); hydrophobic, aromatic amino acids: tryptophan (−3.4), phenylalanine (−2.5), and tyrosine (−2.3).

It is understood that an amino acid can be substituted for another having a similar hydrophilicity and produce a biologically or immunologically modified protein. In such changes, the substitution of amino acids whose hydrophilicity values are within ±2 is preferred, those that are within ±1 are particularly preferred, and those within ±0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

The present disclosure also contemplates isotype modification. By modifying the Fc region to have a different isotype, different functionalities can be achieved. For example, changing to IgG₁ can increase antibody dependent cell cytotoxicity, switching to class A can improve tissue distribution, and switching to class M can improve valency.

Modified antibodies may be made by any technique known to those of skill in the art, including expression through standard molecular biological techniques, or the chemical synthesis of polypeptides. Methods for recombinant expression are addressed elsewhere in this document.

### 2. Fc Region Modifications

The antibodies disclosed herein can also be engineered to include modifications within the Fc region, typically to alter one or more functional properties of the antibody, such as serum half-life, complement fixation, Fc receptor binding, and/or effector function (e.g., antigen-dependent cellular cytotoxicity). Furthermore, the antibodies disclosed herein can be chemically modified (e.g., one or more chemical moieties can be attached to the antibody) or be modified to alter its glycosylation, again to alter one or more functional properties of the antibody. Each of these embodiments is described in further detail below. The numbering of residues in the Fc region is that of the EU index of Kabat. The antibodies disclosed herein also include antibodies with modified (or blocked) Fc regions to provide altered effector functions. See, e.g., U.S. Pat. No. 5,624,821; WO2003/086310; WO2005/120571; WO2006/0057702. Such modification can be used to enhance or suppress various reactions of the immune system, with possible beneficial effects in diagnosis and therapy. Alterations of the Fc region include amino acid changes (substitutions, deletions and insertions), glycosylation or deglycosylation, and adding multiple Fc. Changes to the Fc can also alter the half-life of antibodies in therapeutic antibodies, enabling less frequent dosing and thus increased convenience and decreased use of material. This mutation has been reported to abolish the heterogeneity of inter-heavy chain disulfide bridges in the hinge region.

In one embodiment, the hinge region of CH1 is modified such that the number of cysteine residues in the hinge region is increased or decreased. This approach is described further in U.S. Pat. No. 5,677,425. The number of cysteine residues in the hinge region of CH1 is altered, for example, to facilitate assembly of the light and heavy chains or to increase or decrease the stability of the antibody. In another embodiment, the antibody is modified to increase its biological half-life. Various approaches are possible. For example, one or more of the following mutations can be introduced: T252L, T254S, T256F, as described in U.S. Pat. No. 6,277,375. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022. In yet other embodiments, the Fc region is altered by replacing at least one amino acid residue with a different amino acid residue to alter the effector function(s) of the antibodies. For example, one or more amino acids selected from amino acid residues 234, 235, 236, 237, 297, 318, 320 and 322 can be replaced with a different amino acid residue such that the antibody has an altered affinity for an effector ligand but retains the antigen binding ability of the parent antibody. The effector ligand to which affinity is altered can be, for example, an Fc receptor or the C1 component of complement. This approach is described in further detail in U.S. Pat. Nos. 5,624,821 and 5,648,260.

In another example, one or more amino acid residues within amino acid positions 231 and 239 are altered to thereby alter the ability of the antibody to fix complement. This approach is described further in PCT Publication WO 94/29351. In yet another example, the Fc region is modified to increase or decrease the ability of the antibodies to mediate antibody dependent cellular cytotoxicity (ADCC) and/or to increase or decrease the affinity of the antibodies for an Fcγ receptor by modifying one or more amino acids at the following positions: 238, 239, 243, 248, 249, 252, 254, 255, 256, 258, 264, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 329, 330, 331, 333, 334, 335, 337, 338, 340, 360, 373, 376, 378, 382, 388, 389, 398, 414, 416, 419, 430, 434, 435, 437, 438 or 439. This approach is described further in PCT Publication WO 00/42072. Moreover, the binding sites on human IgG1 for FcγR1, FcγRII, FcγRIII and FcRn have been mapped and variants with improved binding have been described. Specific mutations at positions 256, 290, 298, 333, 334 and 339 were shown to improve binding to FcγRIII. Additionally, the following combination mutants were shown to improve FcγRIII binding: T256A/S298A, S298A/E333A, S298A/K224A and S298A/E333A/K334A.

In one embodiment, the Fc region is modified to decrease the ability of the antibodies to mediate effector function and/or to increase anti-inflammatory properties by modifying residues 243 and 264. In one embodiment, the Fc region of the antibody is modified by changing the residues at positions 243 and 264 to alanine. In one embodiment, the Fc region is modified to decrease the ability of the antibody to mediate effector function and/or to increase anti-inflammatory properties by modifying residues 243, 264, 267 and 328. In still another embodiment, the antibody comprises a particular glycosylation pattern. For example, an aglycosylated antibody can be made (i.e., the antibody lacks glycosylation). The glycosylation pattern of an antibody may be altered to, for example, increase the affinity or avidity of the antibody for an antigen. Such modifications can be accomplished by, for example, altering one or more of the glycosylation sites within the antibody sequence. For example, one or more amino acid substitutions can be made that result removal of one or more of the variable region framework glycosylation sites to thereby eliminate glycosylation at that site. Such aglycosylation may increase the affinity or avidity of the antibody for antigen. See, e.g., U.S. Pat. Nos. 5,714,350 and 6,350,861.

An antibody may also be made in which the glycosylation pattern includes hypofucosylated or afucosylated glycans, such as a hypofucosylated antibodies or afucosylated antibodies have reduced amounts of fucosyl residues on the glycan. The antibodies may also include glycans having an increased amount of bisecting GlcNac structures. Such altered glycosylation patterns have been demonstrated to increase the ADCC ability of antibodies. Such modifications can be accomplished by, for example, expressing the antibodies in a host cell in which the glycosylation pathway was been genetically engineered to produce glycoproteins with particular glycosylation patterns. These cells have been described in the art and can be used as host cells in which to express recombinant antibodies of the invention to thereby produce an antibody with altered glycosylation. For example, the cell lines Ms704, Ms705, and Ms709 lack the fucosyltransferase gene, FUT8 (u (1,6)-fucosyltransferase), such that antibodies expressed in the Ms704, Ms705, and Ms709 cell lines lack fucose on their carbohydrates. The Ms704, Ms705, and Ms709 FUT8^{-/-} cell lines were created by the targeted disruption of the FUT8 gene in CHO/DG44 cells using two replacement vectors (see U.S. Patent Publication No. 20040110704. As another example, EP 1 176 195 describes a cell line with a functionally disrupted FUT8 gene, which encodes a fucosyl transferase, such that antibodies expressed in such a cell line exhibit hypofucosylation by reducing or eliminating the α-1,6 bond-related enzyme. EP 1 176 195 also describes cell lines which have a low enzyme activity for adding fucose to the N-acetylglucosamine that binds to the Fc region of the antibody or does not have the enzyme activity, for example the rat myeloma cell line YB2/0 (ATCC CRL 1662). PCT Publication WO 03/035835 describes a variant CHO cell line, Lec13 cells, with reduced ability to attach fucose to Asn(297)-linked carbohydrates, also resulting in hypofucosylation of antibodies expressed in that host cell. Antibodies with a modified glycosylation profile can also be produced in chicken eggs, as described in PCT Publication WO 06/089231. Alternatively, antibodies with a modified glycosylation profile can be produced in plant cells, such as Lemna (U.S. Pat. No. 7,632,983). Methods for production of antibodies in a plant system are disclosed in the U.S. Pat. Nos. 6,998,267 and 7,388,081. PCT Publication WO 99/54342 describes cell lines engineered to express glycoprotein-modifying glycosyl transferases (e.g., β(1,4)-N-acetylglucosaminyltransferase III (GnTIII)) such that antibodies expressed in the engineered cell lines exhibit increased bisecting GlcNac structures which results in increased ADCC activity of the antibodies.

Alternatively, the fucose residues of the antibodies can be cleaved off using a fucosidase enzyme; e.g., the fucosidase α-L-fucosidase removes fucosyl residues from antibodies. Antibodies disclosed herein further include those produced in lower eukaryote host cells, in particular fungal host cells such as yeast and filamentous fungi have been genetically engineered to produce glycoproteins that have mammalian- or human-like glycosylation patterns. A particular advantage of these genetically modified host cells over currently used mammalian cell lines is the ability to control the glycosylation profile of glycoproteins that are produced in the cells such that compositions of glycoproteins can be produced wherein a particular N-glycan structure predominates (see, e.g., U.S. Pat. Nos. 7,029,872 and 7,449,308). These genetically modified host cells have been used to produce antibodies that have predominantly particular N-glycan structures.

In addition, since fungi such as yeast or filamentous fungi lack the ability to produce fucosylated glycoproteins, antibodies produced in such cells will lack fucose unless the cells are further modified to include the enzymatic pathway for producing fucosylated glycoproteins (See for example, PCT Publication WO2008112092). In particular embodiments, the antibodies disclosed herein further include those produced in lower eukaryotic host cells and which comprise fucosylated and nonfucosylated hybrid and complex N-glycans, including bisected and multiantennary species, including but not limited to N-glycans such as GlcNAc(1-4)Man3GlcNAc2; Gal(1-4)GlcNAc(1-4)Man3GlcNAc2; NANA(1-4)Gal(1-4)GlcNAc(1-4)Man3GlcNAc2. In particular embodiments, the antibody compositions provided herein may comprise antibodies having at least one hybrid N-glycan selected from the group consisting of GlcNAcMan5GlcNAc2; GalGlcNAcMan5GlcNAc2; and NANAGalGlcNAcMan5GlcNAc2. In particular aspects, the hybrid N-glycan is the predominant N-glycan species in the composition. In further aspects, the hybrid N-glycan is a particular N-glycan species that comprises about 30%, 40%, 50%, 60%, 70%, 80%, 90%, 95%, 97%, 98%, 99%, or 100% of the hybrid N-glycans in the composition.

In particular embodiments, the antibody compositions provided herein comprise antibodies having at least one complex N-glycan selected from the group consisting of GlcNAcMan3GlcNAc2; GalGlcNAcMan3GlcNAc2; NANAGalGlcNAcMan3GlcNAc2; GlcNAc2Man3GlcNAc2; GalGlcNAc2Man3GlcNAc2; Gal2GlcNAc2Man3GlcNAc2; NANAGal2GlcNAc2Man3GlcNAc2; and NANA2Gal2GlcNAc2Man3GlcNAc2. In particular aspects, the complex N-glycan is the predominant N-glycan species in the composition. In further aspects, the complex N-glycan is a particular N-glycan species that comprises about 30%, 40%, 50%, 60%, 70%, 80%, 90%, 95%, 97%, 98%, 99%, or 100% of the complex N-glycans in the composition. In particular embodiments, the N-glycan is fusosylated. In general, the fucose is in an α1,3-linkage with the GlcNAc at the reducing end of the N-glycan, an α1,6-linkage with the GlcNAc at the reducing end of the N-glycan, an α1,2-linkage with the Gal at the non-reducing end of the N-glycan, an α1,3-linkage with the GlcNac at the non-reducing end of the N-glycan, or an α1,4-linkage with a GlcNAc at the non-reducing end of the N-glycan.

Therefore, in particular aspects of the above the glycoprotein compositions, the glycoform is in an α1,3-linkage or α1,6-linkage fucose to produce a glycoform selected from the group consisting of Man5GlcNAc2(Fuc), GlcNAcMan5GlcNAc2(Fuc), Man3GlcNAc2(Fuc), GlcNAcMan3GlcNAc2(Fuc), GlcNAc2Man3GlcNAc2(Fuc), GalGlcNAc2Man3GlcNAc2(Fuc), Gal2GlcNAc2Man3GlcNAc2(Fuc), NANAGal2GlcNAc2Man3GlcNAc2(Fuc), and NANA2Gal2GlcNAc2Man3GlcNAc2(Fuc); in an α1,3-linkage or α1,4-linkage fucose to produce a glycoform selected from the group consisting of GlcNAc(Fuc)Man5GlcNAc2, GlcNAc(Fuc)Man3GlcNAc2, GlcNAc2(Fuc1-2)Man3GlcNAc2, GalGlcNAc2(Fuc1-2)Man3GlcNAc2, Gal2GlcNAc2(Fuc1-2)Man3GlcNAc2, NANAGal2GlcNAc2(Fuc1-2)Man3GlcNAc2, and NANA2Gal2GlcNAc2(Fucl-2)Man3GlcNAc2; or in an α1,2-linkage fucose to produce a glycoform selected from the group consisting of Gal(Fuc)GlcNAc2Man3GlcNAc2, Gal2(Fuc1-2)GlcNAc2Man3GlcNAc2, NANAGal2(Fuc1-2)GlcNAc2Man3GlcNAc2, and NANA2Gal2(Fuc1-2)GlcNAc2Man3GlcNAc2.

In further aspects, the antibodies comprise high mannose N-glycans, including but not limited to, Man8GlcNAc2, Man7GlcNAc2, Man6GlcNAc2, Man5GlcNAc2, Man4GlcNAc2, or N-glycans that consist of the Man3GlcNAc2 N-glycan structure. In further aspects of the above, the complex N-glycans further include fucosylated and non-fucosylated bisected and multiantennary species. As used herein, the terms “N-glycan” and “glycoform” are used interchangeably and refer to an N-linked oligosaccharide, for example, one that is attached by an asparagine-N-acetylglucosamine linkage to an asparagine residue of a polypeptide. N-linked glycoproteins contain an N-acetylglucosamine residue linked to the amide nitrogen of an asparagine residue in the protein.

### D. Single Chain Antibodies

A Single Chain Variable Fragment (scFv) is a fusion of the variable regions of the heavy and light chains of immunoglobulins, linked together with a short (usually serine, glycine) linker. This chimeric molecule retains the specificity of the original immunoglobulin, despite removal of the constant regions and the introduction of a linker peptide. This modification usually leaves the specificity unaltered. These molecules were created historically to facilitate phage display where it is highly convenient to express the antigen binding domain as a single peptide. Alternatively, scFv can be created directly from subcloned heavy and light chains derived from a hybridoma. Single chain variable fragments lack the constant Fc region found in complete antibody molecules, and thus, the common binding sites (e.g., protein A/G) used to purify antibodies. These fragments can often be purified/immobilized using Protein L since Protein L interacts with the variable region of kappa light chains.

Flexible linkers generally are comprised of helix- and turn-promoting amino acid residues such as alaine, serine and glycine. However, other residues can function as well. Tang et al. (1996) used phage display as a means of rapidly selecting tailored linkers for single-chain antibodies (scFvs) from protein linker libraries. A random linker library was constructed in which the genes for the heavy and light chain variable domains were linked by a segment encoding an 18-amino acid polypeptide of variable composition. The scFv repertoire (approx. 5×10⁶ different members) was displayed on filamentous phage and subjected to affinity selection with hapten. The population of selected variants exhibited significant increases in binding activity but retained considerable sequence diversity. Screening 1054 individual variants subsequently yielded a catalytically active scFv that was produced efficiently in soluble form. Sequence analysis revealed a conserved proline in the linker two residues after the V_{H} C terminus and an abundance of arginines and prolines at other positions as the only common features of the selected tethers.

The recombinant antibodies of the present disclosure may also involve sequences or moieties that permit dimerization or multimerization of the receptors. Such sequences include those derived from IgA, which permit formation of multimers in conjunction with the J-chain. Another multimerization domain is the Gal4 dimerization domain. In other embodiments, the chains may be modified with agents such as biotin/avidin, which permit the combination of two antibodies.

In a separate embodiment, a single-chain antibody can be created by joining receptor light and heavy chains using a non-peptide linker or chemical unit. Generally, the light and heavy chains will be produced in distinct cells, purified, and subsequently linked together in an appropriate fashion (i.e., the N-terminus of the heavy chain being attached to the C-terminus of the light chain via an appropriate chemical bridge).

Cross-linking reagents are used to form molecular bridges that tie functional groups of two different molecules, e.g., a stabilizing and coagulating agent. However, it is contemplated that dimers or multimers of the same analog or heteromeric complexes comprised of different analogs can be created. To link two different compounds in a step-wise manner, hetero-bifunctional cross-linkers can be used that eliminate unwanted homopolymer formation.

An exemplary hetero-bifunctional cross-linker contains two reactive groups: one reacting with primary amine group (e.g., N-hydroxy succinimide) and the other reacting with a thiol group (e.g., pyridyl disulfide, maleimides, halogens, etc.). Through the primary amine reactive group, the cross-linker may react with the lysine residue(s) of one protein (e.g., the selected antibody or fragment) and through the thiol reactive group, the cross-linker, already tied up to the first protein, reacts with the cysteine residue (free sulfhydryl group) of the other protein (e.g., the selective agent).

It is preferred that a cross-linker having reasonable stability in blood will be employed. Numerous types of disulfide-bond containing linkers are known that can be successfully employed to conjugate targeting and therapeutic/preventative agents. Linkers that contain a disulfide bond that is sterically hindered may prove to give greater stability in vivo, preventing release of the targeting peptide prior to reaching the site of action. These linkers are thus one group of linking agents.

Another cross-linking reagent is SMPT, which is a bifunctional cross-linker containing a disulfide bond that is “sterically hindered” by an adjacent benzene ring and methyl groups. It is believed that steric hindrance of the disulfide bond serves a function of protecting the bond from attack by thiolate anions such as glutathione which can be present in tissues and blood, and thereby help in preventing decoupling of the conjugate prior to the delivery of the attached agent to the target site.

The SMPT cross-linking reagent, as with many other known cross-linking reagents, lends the ability to cross-link functional groups such as the SH of cysteine or primary amines (e.g., the epsilon amino group of lysine). Another possible type of cross-linker includes the hetero-bifunctional photoreactive phenylazides containing a cleavable disulfide bond such as sulfosuccinimidyl-2-(p-azido salicylamido) ethyl-1,3′-dithiopropionate. The N-hydroxy-succinimidyl group reacts with primary amino groups and the phenylazide (upon photolysis) reacts non-selectively with any amino acid residue.

In addition to hindered cross-linkers, non-hindered linkers also can be employed in accordance herewith. Other useful cross-linkers, not considered to contain or generate a protected disulfide, include SATA, SPDP and 2-iminothiolane (Wawrzynczak & Thorpe, 1987). The use of such cross-linkers is well understood in the art. Another embodiment involves the use of flexible linkers.

U.S. Pat. No. 4,680,338, describes bifunctional linkers useful for producing conjugates of ligands with amine-containing polymers and/or proteins, especially for forming antibody conjugates with chelators, drugs, enzymes, detectable labels and the like. U.S. Pat. Nos. 5,141,648 and 5,563,250 disclose cleavable conjugates containing a labile bond that is cleavable under a variety of mild conditions. This linker is particularly useful in that the agent of interest may be bonded directly to the linker, with cleavage resulting in release of the active agent. Particular uses include adding a free amino or free sulfhydryl group to a protein, such as an antibody, or a drug.

U.S. Pat. No. 5,856,456 provides peptide linkers for use in connecting polypeptide constituents to make fusion proteins, e.g., single chain antibodies. The linker is up to about 50 amino acids in length, contains at least one occurrence of a charged amino acid (preferably arginine or lysine) followed by a proline, and is characterized by greater stability and reduced aggregation. U.S. Pat. No. 5,880,270 discloses aminooxy-containing linkers useful in a variety of immunodiagnostic and separative techniques.

### E. Purification

In certain embodiments, the antibodies of the present disclosure may be purified. The term “purified,” as used herein, is intended to refer to a composition, isolatable from other components, wherein the protein is purified to any degree relative to its naturally-obtainable state. A purified protein therefore also refers to a protein, free from the environment in which it may naturally occur. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. Other methods for protein purification include, precipitation with ammonium sulfate, PEG, antibodies and the like or by heat denaturation, followed by centrifugation; gel filtration, reverse phase, hydroxylapatite and affinity chromatography; and combinations of such and other techniques.

In purifying an antibody of the present disclosure, it may be desirable to express the polypeptide in a prokaryotic or eukaryotic expression system and extract the protein using denaturing conditions. The polypeptide may be purified from other cellular components using an affinity column, which binds to a tagged portion of the polypeptide. As is generally known in the art, it is believed that the order of conducting the various purification steps may be changed, or that certain steps may be omitted, and still result in a suitable method for the preparation of a substantially purified protein or peptide.

Commonly, complete antibodies are fractionated utilizing agents (i.e., protein A) that bind the Fc portion of the antibody. Alternatively, antigens may be used to simultaneously purify and select appropriate antibodies. Such methods often utilize the selection agent bound to a support, such as a column, filter or bead. The antibodies is bound to a support, contaminants removed (e.g., washed away), and the antibodies released by applying conditions (salt, heat, etc.).

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. Another method for assessing the purity of a fraction is to calculate the specific activity of the fraction, to compare it to the specific activity of the initial extract, and to thus calculate the degree of purity. The actual units used to represent the amount of activity will, of course, be dependent upon the particular assay technique chosen to follow the purification and whether or not the expressed protein or peptide exhibits a detectable activity.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

### V. Treatment Methods

### A. Formulation and Administration

The present disclosure provides pharmaceutical compositions comprising anti-CHI3L1 antibodies and antigens for generating the same. Such compositions comprise a prophylactically or therapeutically effective amount of an antibody or a fragment thereof, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a particular carrier when the pharmaceutical composition is administered intravenously. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulations can include standard carriers such as pharmaceutical grades of mannitol, lactose, starch, magnesium stearate, sodium saccharine, cellulose, magnesium carbonate, etc. Examples of suitable pharmaceutical agents are described in “Remington's Pharmaceutical Sciences.” Such compositions will contain a prophylactically or therapeutically effective amount of the antibody or fragment thereof, preferably in purified form, together with a suitable amount of carrier so as to provide the form for proper administration to the patient. The formulation should suit the mode of administration, which can be oral, intravenous, intraarterial, intrabuccal, intranasal, nebulized, bronchial inhalation, or delivered by mechanical ventilation.

Antibodies of the present disclosure, as described herein, can be formulated for parenteral administration, e.g., formulated for injection via the intradermal, intravenous, intramuscular, subcutaneous, intra-tumoral or even intraperitoneal routes. The antibodies could alternatively be administered by a topical route directly to the mucosa, for example by nasal drops, inhalation, or by nebulizer. Pharmaceutically acceptable salts, include the acid salts and those which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Passive transfer of antibodies, known as artificially acquired passive immunity, generally will involve the use of intravenous injections. The forms of antibody can be human or animal blood plasma or serum, as pooled human immunoglobulin for intravenous (IVIG) or intramuscular (IG) use, as high-titer human IVIG or IG from immunized or from donors recovering from disease, and as monoclonal antibodies (MAb). Such immunity generally lasts for only a short period of time, and there is also a potential risk for hypersensitivity reactions, and serum sickness, especially from gamma globulin of non-human origin. However, passive immunity provides immediate protection. The antibodies will be formulated in a carrier suitable for injection, i.e., sterile and syringeable.

Generally, the ingredients of compositions of the disclosure are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

### B. Cancer Cell Therapies

In another aspect, the present disclosure provides immune cells which express a chimeric antigen receptor (CAR). In some embodiment, The CAR comprises an antigen-binding fragment provided herein. In an embodiment, the CAR protein includes from the N-terminus to the C-terminus: a leader peptide, an anti-CHI3L1 heavy chain variable domain, a linker domain, an anti-CHI3L1 light chain variable domain, a human IgG1-CH2-CH3 domain, a spacer region, a CD28 transmembrane domain, a 4-1BB intracellular co-stimulatory signaling and a CD3 (intracellular T cell signaling domain.

Also provided are methods for immunotherapy comprising administering an effective amount of the immune cells of the present disclosure. In one embodiments, a medical disease or disorder is treated by transfer of an immune cell population that elicits an immune response. In certain embodiments of the present disclosure, cancer or infection is treated by transfer of an immune cell population that elicits an immune response. Provided herein are methods for treating or delaying progression of cancer in an individual comprising administering to the individual an effective amount an antigen-specific cell therapy.

The immune cells may be T cells (e.g., regulatory T cells, CD4+ T cells, CD8+ T cells, or gamma-delta T cells), NK cells, invariant NK cells, NKT cells, or macrophages. Also provided herein are methods of producing and engineering the immune cells as well as methods of using and administering the cells for adoptive cell therapy, in which case the cells may be autologous or allogeneic. Thus, the immune cells may be used as immunotherapy, such as to target cancer cells.

The immune cells may be isolated from subjects, particularly human subjects. The immune cells can be obtained from healthy human subjects, healthy volunteers, or healthy donors. The immune cells can be obtained from a subject of interest, such as a subject suspected of having a particular disease or condition, a subject suspected of having a predisposition to a particular disease or condition, or a subject who is undergoing therapy for a particular disease or condition. Immune cells can be collected from any location in which they reside in the subject including, but not limited to, blood, cord blood, spleen, thymus, lymph nodes, and bone marrow. The isolated immune cells may be used directly, or they can be stored for a period of time, such as by freezing.

The immune cells may be enriched/purified from any tissue where they reside including, but not limited to, blood (including blood collected by blood banks or cord blood banks), spleen, bone marrow, tissues removed and/or exposed during surgical procedures, and tissues obtained via biopsy procedures. Tissues/organs from which the immune cells are enriched, isolated, and/or purified may be isolated from both living and non-living subjects, wherein the non-living subjects are organ donors. In particular embodiments, the immune cells are isolated from blood, such as peripheral blood or cord blood. In some aspects, immune cells isolated from cord blood have enhanced immunomodulation capacity, such as measured by CD4- or CD8-positive T cell suppression. In specific aspects, the immune cells are isolated from pooled blood, particularly pooled cord blood, for enhanced immunomodulation capacity. The pooled blood may be from 2 or more sources, such as 3, 4, 5, 6, 7, 8, 9, 10 or more sources (e.g., donor subjects).

The population of immune cells can be obtained from a subject in need of therapy or suffering from a disease associated with reduced immune cell activity. Thus, the cells will be autologous to the subject in need of therapy. Alternatively, the population of immune cells can be obtained from a donor, preferably a histocompatibility matched donor. The immune cell population can be harvested from the peripheral blood, cord blood, bone marrow, spleen, or any other organ/tissue in which immune cells reside in said subject or donor. The immune cells can be isolated from a pool of subjects and/or donors, such as from pooled cord blood.

When the population of immune cells is obtained from a donor distinct from the subject, the donor is preferably allogeneic, provided the cells obtained are subject-compatible in that they can be introduced into the subject. Allogeneic donor cells are may or may not be human-leukocyte-antigen (HLA)-compatible. To be rendered subject-compatible, allogeneic cells can be treated to reduce immunogenicity.

The immune cells can be genetically engineered to express antigen receptors such as engineered TCRs and/or chimeric antigen receptors (CARs). For example, the host cells (e.g., autologous or allogeneic T-cells) are modified to express a T cell receptor (TCR) having antigenic specificity for a cancer antigen. In particular embodiments, NK cells are engineered to express a TCR. The NK cells may be further engineered to express a CAR. Multiple CARs and/or TCRs, such as to different antigens, may be added to a single cell type, such as T cells or NK cells.

Suitable methods of modification are known in the art. See, for instance, Sambrook et al., supra; and Ausubel et al., CURRENT PROTOCOLS IN MOLECULAR BIOLOGY, Greene Publishing Associates and John Wiley & Sons, N Y, 1994. For example, the cells may be transduced to express a T cell receptor (TCR) having antigenic specificity for a cancer antigen using transduction techniques described in Heemskerk et al. (2008) and Johnson et al. (2009).

In some embodiments, the cells comprise one or more nucleic acids introduced via genetic engineering that encode one or more antigen receptors, and genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature (e.g., chimeric).

### C. Cancer Combination Therapies

It may also be desirable to provide combination treatments using antibodies of the present disclosure in conjunction with additional anti-cancer therapies. These therapies would be provided in a combined amount effective to achieve a reduction in one or more disease parameter. This process may involve contacting the cells/subjects with the both agents/therapies at the same time, e.g., using a single composition or pharmacological formulation that includes both agents, or by contacting the cell/subject with two distinct compositions or formulations, at the same time, wherein one composition includes the antibody and the other includes the other agent.

Alternatively, the antibody may precede or follow the other treatment by intervals ranging from minutes to weeks. One would generally ensure that a significant period of time did not expire between the time of each delivery, such that the therapies would still be able to exert an advantageously combined effect on the cell/subject. In such instances, it is contemplated that one would contact the cell with both modalities within about 12-24 hours of each other, within about 6-12 hours of each other, or with a delay time of only about 12 hours. In some situations, it may be desirable to extend the time period for treatment significantly; however, where several 10 days (2, 3, 4, 5, 6 or 7) to several weeks (1, 2, 3, 4, 5, 6, 7 or 8) lapse between the respective administrations.

It also is conceivable that more than one administration of either the anti-CH3L1 antibody or the other therapy will be desired. Various combinations may be employed, where the antibody is “A,” and the other therapy is “B,” as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are contemplated. To kill cells, inhibit cell growth, inhibit metastasis, inhibit angiogenesis or otherwise reverse or reduce the malignant phenotype of tumor cells, using the methods and compositions of the present invention, one may contact a target cell or site with an antibody and at least one other therapy. These therapies would be provided in a combined amount effective to kill or inhibit proliferation of cancer cells. This process may involve contacting the cells/site/subject with the agents/therapies at the same time.

Particular agents contemplated for combination therapy with antibodies of the present disclosure include chemotherapy and hematopoietic stem cell transplantation. Chemotherapy may include cytarabine (ara-C) and an anthracycline (most often daunorubicin), high-dose cytarabine alone, all-trans-retinoic acid (ATRA) in addition to induction chemotherapy, usually an anthracycline, histamine dihydrochloride (Ceplene) and interleukin 2 (Proleukin) after the completion of consolidation therapy, gemtuzumab ozogamicin (Mylotarg) for patients aged more than 60 years with relapsed AML who are not candidates for high-dose chemotherapy, clofarabine, as well as targeted therapies, such as kinase inhibitors, farnesyl transferase inhibitors, decitabine, and inhibitors of MDR1 (multidrug-resistance protein), or arsenic trioxide or relapsed acute promyelocytic leukemia (APL).

In certain embodiments, the agents for combination therapy are one or more drugs selected from the group consisting of a topoisomerase inhibitor, an anthracycline topoisomerase inhibitor, an anthracycline, a daunorubicin, a nucleoside metabolic inhibitor, a cytarabine, a hypomethylating agent, a low dose cytarabine (LDAC), a combination of daunorubicin and cytarabine, a daunorubicin and cytarabine liposome for injection, Vyxeos®, an azacytidine, Vidaza®, a decitabine, an all-trans-retinoic acid (ATRA), an arsenic, an arsenic trioxide, a histamine dihydrochloride, Ceplene®, an interleukin-2, an aldesleukin, Proleukin®, a gemtuzumab ozogamicin, Mylotarg®, an FLT-3 inhibitor, a midostaurin, Rydapt®, a clofarabine, a farnesyl transferase inhibitor, a decitabine, an IDH1 inhibitor, an ivosidenib, Tibsovo®, an IDH2 inhibitor, an enasidenib, Idhifa®, a smoothened (SMO) inhibitor, a glasdegib, an arginase inhibitor, an IDO inhibitor, an epacadostat, a BCL-2 inihbitor, a venetoclax, Venclexta®, a platinum complex derivative, oxaliplatin, a kinase inhibitor, a tyrosine kinase inhibitor, a PI3 kinase inhibitor, a BTK inhibitor, an ibrutinib, IMBRUVICA®, an acalabrutinib, CALQUENCE®, a zanubrutinib, a PD-1 antibody, a PD-L1 antibody, a CTLA-4 antibody, a LAG3 antibody, an ICOS antibody, a TIGIT antibody, a TIM3 antibody, a CD40 antibody, a 4-1BB antibody, a CD47 antibody, a SIRPla antibody or fusions protein, an antagonist of E-selectin, an antibody binding to a tumor antigen, an antibody binding to a T-cell surface marker, an antibody binding to a myeloid cell or NK cell surface marker, an alkylating agent, a nitrosourea agent, an antimetabolite, an antitumor antibiotic, an alkaloid derived from a plant, a hormone therapy medicine, a hormone antagonist, an aromatase inhibitor, and a P-glycoprotein inhibitor.

### D. Hepatotoxicity Combination Therapies

It may also be desirable to provide combination treatments using antibodies of the present disclosure in conjunction with additional hepatotoxicity therapies. These therapies would be provided in a combined amount effective to achieve a reduction in one or more disease parameter. This process may involve contacting the cells/subjects with the both agents/therapies at the same time, e.g., using a single composition or pharmacological formulation that includes both agents, or by contacting the cell/subject with two distinct compositions or formulations, at the same time, wherein one composition includes the antibody and the other includes the other agent.

Alternatively, the antibody may precede or follow the other treatment by intervals ranging from minutes to weeks. One would generally ensure that a significant period of time did not expire between the time of each delivery, such that the therapies would still be able to exert an advantageously combined effect on the cell/subject. In such instances, it is contemplated that one would contact the cell with both modalities within about 12-24 hours of each other, within about 6-12 hours of each other, or with a delay time of only about 12 hours. In some situations, it may be desirable to extend the time period for treatment significantly; however, where several 10 days (2, 3, 4, 5, 6 or 7) to several weeks (1, 2, 3, 4, 5, 6, 7 or 8) lapse between the respective administrations.

It also is conceivable that more than one administration of either the anti-CH3L1 antibody or the other therapy will be desired. Various combinations may be employed, where the antibody is “A,” and the other therapy is “B,” as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are contemplated. This process may involve contacting the cells/site/subject with the agents/therapies at the same time or at different times. The other therapy may be supportive care, including pain medication and fluids, and in some instances an anti-toxin.

### VI. Antibody Conjugates

Antibodies of the present disclosure may be linked to at least one agent to form an antibody conjugate. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules which have been attached to antibodies include toxins, anti-tumor agents, therapeutic enzymes, radionuclides, antiviral agents, chelating agents, cytokines, growth factors, and oligo- or polynucleotides. By contrast, a reporter molecule is defined as any moiety which may be detected using an assay. Non-limiting examples of reporter molecules which have been conjugated to antibodies include enzymes, radiolabels, haptens, fluorescent labels, phosphorescent molecules, chemiluminescent molecules, chromophores, photoaffinity molecules, colored particles or ligands, such as biotin.

Antibody-drug conjugates have emerged as a breakthrough approach to the development of cancer therapeutics. Antibody-drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment (Leal et al., 2014). As antibody engineering and linker-payload optimization are becoming more and more mature, the discovery and development of new ADCs are increasingly dependent on the identification and validation of new targets that are suitable to this approach and the generation of targeting MAbs. Two criteria for ADC targets are upregulated/high levels of expression in tumor cells and robust internalization.

Antibody conjugates are also preferred for use as diagnostic agents. Antibody diagnostics generally fall within two classes, those for use in in vitro diagnostics, such as in a variety of immunoassays, and those for use in vivo diagnostic protocols, generally known as “antibody-directed imaging.” Many appropriate imaging agents are known in the art, as are methods for their attachment to antibodies (see, for e.g., U.S. Pat. Nos. 5,021,236, 4,938,948, and 4,472,509). The imaging moieties used can be paramagnetic ions, radioactive isotopes, fluorochromes, NMR-detectable substances, and X-ray imaging agents.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for therapeutic and/or diagnostic application, one might mention astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and/or yttrium⁹⁰. ¹²⁵I is often being preferred for use in certain embodiments, and technicium^{99m} and/or indium¹¹¹ are also often preferred due to their low energy and suitability for long range detection. Radioactively labeled monoclonal antibodies of the present disclosure may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the disclosure may be labeled with technetium^{99m} by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA).

Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Another type of antibody conjugates contemplated in the present disclosure are those intended primarily for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. Preferred secondary binding ligands are biotin and avidin and streptavidin compounds. The use of such labels is well known to those of skill in the art and are described, for example, in U.S. Pat. Nos. 3,817,837, 3,850,752, 3,939,350, 3,996,345, 4,277,437, 4,275,149 and 4,366,241.

Yet another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this may not be advantageous since it results in loss of antigen binding by the antibody conjugate.

Molecules containing azido groups may also be used to form covalent bonds to proteins through reactive nitrene intermediates that are generated by low intensity ultraviolet light (Potter and Haley, 1983). In particular, 2- and 8-azido analogues of purine nucleotides have been used as site-directed photoprobes to identify nucleotide binding proteins in crude cell extracts (Owens & Haley, 1987; Atherton et al., 1985). The 2- and 8-azido nucleotides have also been used to map nucleotide binding domains of purified proteins (Khatoon et al., 1989; King et al., 1989; Dholakia et al., 1989) and may be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the antibody (U.S. Pat. Nos. 4,472,509 and 4,938,948). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

In other embodiments, derivatization of immunoglobulins by selectively introducing sulfhydryl groups in the Fc region of an immunoglobulin, using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region have also been disclosed in the literature (O'Shannessy et al., 1987). This approach has been reported to produce diagnostically and therapeutically promising antibodies which are currently in clinical evaluation.

### VII. Immunodetection Methods

In still further embodiments, the present disclosure concerns immunodetection methods for binding, purifying, removing, quantifying and otherwise generally detecting CHI3L1-related cancers. While such methods can be applied in a traditional sense, another use will be in quality control and monitoring of vaccine stocks, where antibodies according to the present disclosure can be used to assess the amount or integrity (i.e., long term stability) of antigens. Alternatively, the methods may be used to screen various antibodies for appropriate/desired reactivity profiles.

Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. In particular, a competitive assay for the detection and quantitation of CHI3L1 also is provided. The steps of various useful immunodetection methods have been described in the scientific literature, such as, e.g., Doolittle and Ben-Zeev (1999), Gulbis and Galand (1993), De Jager et al. (1993), and Nakamura et al. (1987). In general, the immunobinding methods include obtaining a sample suspected of containing CHI3L1-related cancers, and contacting the sample with a first antibody in accordance with the present disclosure, as the case may be, under conditions effective to allow the formation of immunocomplexes.

These methods include methods for detecting or purifying CHI3L1 or CHI3L1-related cancer cells from a sample. The antibody will preferably be linked to a solid support, such as in the form of a column matrix, and the sample suspected of containing the CHI3L1-related cancer cells will be applied to the immobilized antibody. The unwanted components will be washed from the column, leaving the CHI3L1-expressing cells immunocomplexed to the immobilized antibody, which is then collected by removing the organism or antigen from the column.

The immunobinding methods also include methods for detecting and quantifying the amount of CHI3L1-related cancer cells or related components in a sample and the detection and quantification of any immune complexes formed during the binding process. Here, one would obtain a sample suspected of containing CHI3L1-related cancer cells and contact the sample with an antibody that binds CHI3L1 or components thereof, followed by detecting and quantifying the amount of immune complexes formed under the specific conditions. In terms of antigen detection, the biological sample analyzed may be any sample that is suspected of containing CHI3L1-related cancers, such as a tissue section or specimen, a homogenized tissue extract, a biological fluid, including blood and serum, or a secretion, such as feces or urine.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to CHI3L1. After this time, the sample-antibody composition, such as a tissue section, ELISA plate, dot blot or Western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. Patents concerning the use of such labels include U.S. Pat. Nos. 3,817,837, 3,850,752, 3,939,350, 3,996,345, 4,277,437, 4,275,149 and 4,366,241. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two-step approach. A second binding ligand, such as an antibody that has binding affinity for the antibody, is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection uses two different antibodies. A first biotinylated antibody is used to detect the target antigen, and a second antibody is then used to detect the biotin attached to the complexed biotin. In that method, the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

### A. ELISAs

Immunoassays, in their most simple and direct sense, are binding assays. Certain preferred immunoassays are the various types of enzyme linked immunosorbent assays (ELISAs) and radioimmunoassays (RIA) known in the art. Immunohistochemical detection using tissue sections is also particularly useful. However, it will be readily appreciated that detection is not limited to such techniques, and western blotting, dot blotting, FACS analyses, and the like may also be used.

In one exemplary ELISA, the antibodies of the disclosure are immobilized onto a selected surface exhibiting protein affinity, such as a well in a polystyrene microtiter plate. Then, a test composition suspected of containing the CHI3L1-related cancer cells is added to the wells. After binding and washing to remove non-specifically bound immune complexes, the bound antigen may be detected. Detection may be achieved by the addition of another anti-CHI3L1 antibody that is linked to a detectable label. This type of ELISA is a simple “sandwich ELISA.” Detection may also be achieved by the addition of a second anti-CHI3L1 antibody, followed by the addition of a third antibody that has binding affinity for the second antibody, with the third antibody being linked to a detectable label.

In another exemplary ELISA, the samples suspected of containing the CHI3L1-related cancer cells are immobilized onto the well surface and then contacted with the anti-CHI3L1 antibodies of the disclosure. After binding and washing to remove non-specifically bound immune complexes, the bound anti-CHI3L1 antibodies are detected. Where the initial anti-CHI3L1 antibodies are linked to a detectable label, the immune complexes may be detected directly. Again, the immune complexes may be detected using a second antibody that has binding affinity for the first anti-CHI3L1 antibody, with the second antibody being linked to a detectable label.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. Preferably, this will be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

### B. Western Blot

The Western blot (alternatively, protein immunoblot) is an analytical technique used to detect specific proteins in a given sample of tissue homogenate or extract. It uses gel electrophoresis to separate native or denatured proteins by the length of the polypeptide (denaturing conditions) or by the 3-D structure of the protein (native/non-denaturing conditions). The proteins are then transferred to a membrane (typically nitrocellulose or PVDF), where they are probed (detected) using antibodies specific to the target protein.

Samples may be taken from whole tissue or from cell culture. In most cases, solid tissues are first broken down mechanically using a blender (for larger sample volumes), using a homogenizer (smaller volumes), or by sonication. Cells may also be broken open by one of the above mechanical methods. Assorted detergents, salts, and buffers may be employed to encourage lysis of cells and to solubilize proteins. Protease and phosphatase inhibitors are often added to prevent the digestion of the sample by its own enzymes. Tissue preparation is often done at cold temperatures to avoid protein denaturing.

The proteins of the sample are separated using gel electrophoresis. Separation of proteins may be by isoelectric point (pI), molecular weight, electric charge, or a combination of these factors. The nature of the separation depends on the treatment of the sample and the nature of the gel. This is a very useful way to determine a protein. It is also possible to use a two-dimensional (2-D) gel which spreads the proteins from a single sample out in two dimensions. Proteins are separated according to isoelectric point (pH at which they have neutral net charge) in the first dimension, and according to their molecular weight in the second dimension.

In order to make the proteins accessible to antibody detection, they are moved from within the gel onto a membrane made of nitrocellulose or polyvinylidene difluoride (PVDF). The membrane is placed on top of the gel, and a stack of filter papers placed on top of that. The entire stack is placed in a buffer solution which moves up the paper by capillary action, bringing the proteins with it. Another method for transferring the proteins is called electroblotting and uses an electric current to pull proteins from the gel into the PVDF or nitrocellulose membrane. The proteins move from within the gel onto the membrane while maintaining the organization they had within the gel. As a result of this blotting process, the proteins are exposed on a thin surface layer for detection (see below). Both varieties of membrane are chosen for their non-specific protein binding properties (i.e., binds all proteins equally well). Protein binding is based upon hydrophobic interactions, as well as charged interactions between the membrane and protein. Nitrocellulose membranes are cheaper than PVDF, but are far more fragile and do not stand up well to repeated probings. The uniformity and overall effectiveness of transfer of protein from the gel to the membrane can be checked by staining the membrane with Coomassie Brilliant Blue or Ponceau S dyes. Once transferred, proteins are detected using labeled primary antibodies, or unlabeled primary antibodies followed by indirect detection using labeled protein A or secondary labeled antibodies binding to the Fc region of the primary antibodies.

### C. Immunohistochemistry

The antibodies of the present disclosure may also be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors, and is well known to those of skill in the art (Brown et al., 1990; Abbondanzo et al., 1990; Allred et al., 1990).

Briefly, frozen-sections may be prepared by rehydrating 50 ng of frozen “pulverized” tissue at room temperature in phosphate buffered saline (PBS) in small plastic capsules; pelleting the particles by centrifugation; resuspending them in a viscous embedding medium (OCT); inverting the capsule and/or pelleting again by centrifugation; snap-freezing in −70° C. isopentane; cutting the plastic capsule and/or removing the frozen cylinder of tissue; securing the tissue cylinder on a cryostat microtome chuck; and/or cutting 25-50 serial sections from the capsule. Alternatively, whole frozen tissue samples may be used for serial section cuttings.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections. Again, whole tissue samples may be substituted.

### D. Immunodetection Kits

In still further embodiments, the present disclosure concerns immunodetection kits for use with the immunodetection methods described above. As the antibodies may be used to detect CHI3L1-related cancer cells, the antibodies may be included in the kit. The immunodetection kits will thus comprise, in suitable container means, a first antibody that binds to an CHI3L1, and optionally an immunodetection reagent.

In certain embodiments, the antibody may be pre-bound to a solid support, such as a column matrix and/or well of a microtitre plate. The immunodetection reagents of the kit may take any one of a variety of forms, including those detectable labels that are associated with or linked to the given antibody. Detectable labels that are associated with or attached to a secondary binding ligand are also contemplated. Exemplary secondary ligands are those secondary antibodies that have binding affinity for the first antibody.

Further suitable immunodetection reagents for use in the present kits include the two-component reagent that comprises a secondary antibody that has binding affinity for the first antibody, along with a third antibody that has binding affinity for the second antibody, the third antibody being linked to a detectable label. As noted above, a number of exemplary labels are known in the art and all such labels may be employed in connection with the present disclosure.

The kits may further comprise a suitably aliquoted composition of CHI3L1, whether labeled or unlabeled, as may be used to prepare a standard curve for a detection assay. The kits may contain antibody-label conjugates either in fully conjugated form, in the form of intermediates, or as separate moieties to be conjugated by the user of the kit. The components of the kits may be packaged either in aqueous media or in lyophilized form.

The container means of the kits will generally include at least one vial, test tube, flask, bottle, syringe or other container means, into which the antibody may be placed, or preferably, suitably aliquoted. The kits of the present disclosure will also typically include a means for containing the antibody, antigen, and any other reagent containers in close confinement for commercial sale. Such containers may include injection or blow-molded plastic containers into which the desired vials are retained.

### E. Flow Cytometry and FACS

The antibodies of the present disclosure may also be used in flow cytometry or FACS. Flow cytometry is a laser- or impedance-based technology employed in many detection assays, including cell counting, cell sorting, biomarker detection and protein engineering. The technology suspends cells in a stream of fluid and passing them through an electronic detection apparatus, which allows simultaneous multiparametric analysis of the physical and chemical characteristics of up to thousands of particles per second. Flow cytometry is routinely used in the diagnosis disorders, especially blood cancers, but has many other applications in basic research, clinical practice and clinical trials.

Fluorescence-activated cell sorting (FACS) is a specialized type of cytometry. It provides a method for sorting a heterogenous mixture of biological cells into two or more containers, one cell at a time, based on the specific light scattering and fluorescent characteristics of each cell. In general, the technology involves a cell suspension entrained in the center of a narrow, rapidly flowing stream of liquid. The flow is arranged so that there is a large separation between cells relative to their diameter. A vibrating mechanism causes the stream of cells to break into individual droplets. Just before the stream breaks into droplets, the flow passes through a fluorescence measuring station where the fluorescence of each cell is measured. An electrical charging ring is placed just at the point where the stream breaks into droplets. A charge is placed on the ring based immediately prior to fluorescence intensity being measured, and the opposite charge is trapped on the droplet as it breaks form the stream. The charged droplets then fall through an electrostatic deflection system that diverts droplets into containers based upon their charge.

In certain embodiments, to be used in flow cytometry or FACS, the antibodies of the present disclosure are labeled with fluorophores and then allowed to bind to the cells of interest, which are analyzed in a flow cytometer or sorted by a FACS machine.

### VIII. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Materials and Methods

Animal experiments and procedures. C57BL/6J and CD44^{-/-} mice were purchased from the Jackson Laboratory and colonies were maintained at the animal core facility of University of Texas Health Science Center (UTHealth). C57BL/6J, not C57BL/6N, was used as WT control because both CHI3L1^{-/-} and CD44^{-/-} mice are on the C57BL/6J background, determined by PCR (data not shown). CHI3L1^{-/-} mice were provided by Dr. Jack A Elias (Brown University, Providence, RI, United States). All mice were maintained. Animal studies described in this manuscript has been approved and conducted under the oversight of the UTHealth Institutional Animal Care and Use Committee (IACUC). Mice (8-12 weeks old) were starved overnight (5:00 μm to 9:00 am) and injected intraperitoneally (i.p.) with APAP (Sigma, A7085) at a dose of 210 mg/kg for male mice and 325 mg/kg for female mice, as female mice are less susceptible to APAP-induced liver injury.55 Regarding sex as a biological variable, male mice have been the choice in the vast majority of the studies reported in the literature.^{7,54,56} However, Male and female mice respond to APAP treatment similarly, although their dose responses are different (ED FIG. 7). In some experiments, mice received APAP were immediately injection intraperitoneally (i.p.) with either PBS (100 μl) or recombinant mouse CHI3L1 (rCHI3L1, 500 ng/mouse in 100 μl, Sino Biological 50929-M08H). Liver frozen sections were harvested at time point indicated in the figure legends and immunofluorescence staining was performed to detect KCs, LSECs and platelets using anti-F4/80, anti-CD31 and anti-CD41 antibodies, respectively. Liver paraffin sections and serum were harvested at time point indicated in the figure legends and H&E staining, ALT measurements were performed to examine liver injury.

Blocking endogenous CD44. Mice were injected i.p with control (Ctrl) IgG (BD Pharmingen, 559478, 50 μg/mouse) or anti-CD44 antibody (BD Pharmingen, 553131, 50 μg/mouse) in CHI3L1^{-/-} reconstituted with rCHI3L1 at 30 min prior to APAP treatment or Con A treatment.

Blocking endogenous Podoplanin. Mice were injected i.v. (with Ctrl IgG (Bioxcell InvivoMab, BE0087, 100 μg/mouse) or anti-Podoplanin antibody (Bioxcell InvivoMab, BE0236, 100 μg/mouse) in CHI3L1^{-/-} reconstituted with rCHI3L1 at 16 h prior to APAP treatment.

Platelet depletion. WT mice were injected i.v. with Ctrl IgG (BD Pharmingen, 553922, 2 mg/kg) or CD41 antibody (BD Pharmingen, 553847, 2 mg/kg) to deplete platelets at 12 h prior to APAP treatment or Con A treatment.

KC depletion. WT mice were injected i.v. with empty liposomes (PBS, 100 μl/mouse) or clodronate-containing liposomes (CLDN, 100 μl/mouse) to deplete KCs at 9 h prior to APAP treatment or Con A treatment. Clodronate liposomes were generated as previously described⁵⁴.

Evaluation of the effects of anti-CHI3L1 monoclonal antibodies. To examine the therapeutic potential of anti-mouse CHI3L1 mAbs, WT mice were treated with APAP and after 3 h injected (i.p.) with either Ctrl IgG or anti-mouse CHI3L1 antibody clones (C15, C43, C53, C61, C64, C55, C59). To examine the therapeutic potential of anti-human CHI3L1 mAbs, CHI3L1^{-/-} mice treated with APAP were immediately injected (i.p.) with either PBS (100 μl) or recombinant human CHI3L1 (rhCHI3L1, 1 μg/mouse in 100 μl, Sino Biological 11227-H08H). After 3 h, these mice were then divided into two groups that were injected (i.p.) with either Ctrl IgG or anti-human CHI3L1 mAbs C7.

Measurements of alanine aminotransferase (ALT) and cytokine levels. Serum ALT levels were measured using a diagnostic assay kit (Teco Dignostics, Anaheim CA). The levels of IL-4, IL-13, IL-10, IL-6, IFN-γ and CHI3L1 in serum and liver tissue samples were determined by sandwich enzyme-linked immunosorbent assay (ELISA, R&D) according to the manufacturer's protocol.

Preparation of liver cells and in vitro cell culture. Hepatic nonparenchymal cells (NPCs) and hepatocytes were isolated as previously described⁵⁷. In brief, mice were anesthetized and liver tissues were perfused with EGTA solution, followed by a 0.04% collagenase digestion buffer. After digestion, gall bladder was removed, and digested liver was cut into small pieces to undergo tubing digestion. Live hepatocytes and NPCs were further isolated by gradient centrifugation by percoll (Sigma) and Optiprep (Stemcell). In order to further obtain purified LSEC and KCs, LSEC and KCs fractions were stained with phycoerythrin (PE)-conjugated anti-CD146 (for LSEC, Invitrogen, 12-1469-42), and anti-F4/80 (for KCs, Invitrogen, 12-4801-82) antibodies, respectively and LSEC and KCs were positively selected using EasySep™ Mouse PE Positive Selection Kit (Stemcell technologies) following manufacturers' instructions. Each subset will yield a purity around 90%.

Co-culture of KCs and washed platelets. Isolated KCs were cultured in DMEM with 10% fetal bovine serum and pre-treated with Podoplanin antibody (Bioxcell InvivoMab, BE0236, 2 μg/ml) for 30 mins and then co-culture with washed platelets for 30 mins. Wash out unbound platelets and stain Podoplanin and Clec-2 on KCs.

Isolation of washed platelets. Mouse whole blood was collected with anti-coagulant ACD solution from Inferior vena cava. Platelets were further isolated by additional washes of Tyrode's buffer. Isolated washed platelets were subject for functional assay after incubation with PGI2 (Sigma, P6188) for 30 mins.

Flow cytometry. Isolated NPCs were further incubated with 2 μg rCHI3L1 for 2 h. After wash, resuspended cell pellets were first incubated with 1 μl anti-mouse FcγRII/III (Becton Dickinson, Franklin Lakes, NJ, USA) to minimize non-specific antibody binding and then stained with Anti-mouse CD45-V655 (eBioscience, 15520837), F4/80-APC/Cy7 (Biolegend, 123118), Ly6C-APC (BD Pharmingen, 560595), Ly6G-V450 (BD Pharmingen, 560603), CD146-PerCP-Cy5.5 (BD Pharmingen, 562134), CD44-PE (BD Pharmingen, 553134), Anti-6× His tag-FITC (abcam, ab1206). Cells were analysed on a CytoFLEX LX Flow Cytometer (Beckman coulter, IN, USA) using FlowJo software (Tree Star, Ashland, OR, USA). For flow cytometric analysis, CD45+ cells were gated to exclude endothelial cells, hepatic stellate cells, and residue hepatocytes. Within CD45+ cells, CD44+ cells that bind CHI3L1 were back gated to determine cells types.

Extraction of liver proteins, immunoprecipitation, and mass spectrometry. Snap frozen liver tissues were pulverized to extract liver proteins in STE buffer. Protein concentration was measured by BCA kit (Thermo Scientific, 23225) following instructions.

For immunoprecipitation in Kupffer cells lysis. Proteins were extracted from KCs lysis and incubated with 5 μg mouse rCHI3L1, following by immunoprecipitation with 2 μg Rabbit IgG (negative control, Peprotech, 500-p00) or 2 μg anti-his tag antibody (Abnova, MAB12807). Dynabeads Protein G (Invitrogen, 1003D) were used to pull down antibodies-binding proteins. Immunoprecipitated proteins were subject to mass spectrometry analysis in proteomics core facility at UTHealth.

For immunoprecipitation of liver homogenates. 10 mg liver proteins extracted from CD44^{-/-} and WT mice treated with APAP for 2 h were incubated with 5 μg rCHI3L1, followed by immunoprecipitation with 2 μg anti-CD44 antibody (BD Pharmingen, 553131). Dynabeads Protein G (Invitrogen, 1003D) were used to pull down antibodies-binding proteins. Input protein and immunoprecipitated proteins were subject to western blot analysis.

For in vitro immunoprecipitation assay. 2 μg rhCHI3L1(Sino Biological, His Tag, 11227-H08H) or 2 μg human Ctrl protein were incubated with 2 μg human CD44 (Sino Biological, Fc Tag, 12211-H02H) and immunoprecipitated with 2 μg anti-his tag antibody (Abnova, MAB12807), followed by pull-down by Dynabeads Protein G (Invitrogen, 1003D). Input protein and immunoprecipitated proteins were subject to western blot analysis.

Western blotting. Samples were prepared with loading buffer and boiled to load onto SDS-PAGE gels. Nitrocellulose membranes (Bio-Rad) were used to transfer proteins. Primary antibodies used to detect specific proteins include anti-CHI3L1 (Proteintech, 12036-1-AP, 1:1000), anti-CD44 (abcam, ab25340, 1:500), anti-R-actin (Cell Signaling, 4970, 1:1000), anti-his tag (Abnova, MAB12807, 1:1000), anti-Cyp2e1 (LifeSpan BioSciences, LS-C6332, 1:500), anti-APAP adducts54 (provided by Dr. Lance R. Pohl, NIH, 1:500). Secondary antibodies include Goat anti-Rabbit IgG (Jackson ImmunoResearch, 111-035-144, 1:1000), Goat anti-Rat (Jackson ImmunoResearch, 112-035-003, 1:1000).

Bio-layer interferometry. The binding affinity between CD44-Fc and His-CHI3L1 was measured by using in the Octet system 8-channel Red96 (Menlo Park). Protein A biosensors and kinetics buffer were purchased from Pall Life Sciences (Menlo Park). CD44-Fc protein was immobilized onto Protein A biosensors and incubated with varying concentrations of recombinant His-CHI3L1 in solution (1000 nM to 1.4 nM). Binding kinetic constants were determined using 1:1 fitting model with ForteBio's data analysis software 7.0, and the KD was calculated using the ratio koff/kon (the highest 4 concentrations were used to calculate the KD.).

Immunohistochemical (IHC) and immunofluorescent (IF) stainings.

H&E and IHC stainings were performed on paraffin sections using the following antibodies: anti-human CD41 (Proteintech, 24552-2-AP, 1:200), anti-human CD68 (Thermo Fisher, MA5-13324, 1:100), anti-human CHI3L1 (Proteintech, 12036-1-AP, 1:100), anti-mouse F4/80 (Bio Rad, MCA497R, 1:200). IF staining were performed on frozen sections using the following antibodies: anti-mouse CD41 (BD Bioscience, Clone MWReg 30), anti-CD31 (Biolegend, 102516, 1:100), mouse F4/80 (Biolegend, 123122, 1:100), anti-CD44 (abcam, clone KM81, ab112178, 1:200), anti-CHI3L1 (Proteintech, 12036-1-AP, 1:100), anti-Podoplanin (Novus, biological, NB600-1015, 1:100), anti-Clec-2 (Biorbyt, orb312182, 1:100). Alexa 488-conjugated donkey anti-rat immunoglobulin (Invitrogen, A-21208, 1:1000) was used as a secondary antibody for CD41, CD44 detection. Alexa 488-conjugated goat anti-rabbit immunoglobulin (Invitrogen, A-11034, 1:1000) was used as a secondary antibody for Clec-2 detection. Alexa 594-conjugated goat anti-rabbit immunoglobulin (Invitrogen, A-11012, 1:1000) was used as a secondary antibody for CHI3L1 detection. Alexa 594-conjugated goat anti-hamster immunoglobulin (Invitrogen, A-21113, 1:1000) was used as a secondary antibody for Podoplanin detection. Nuclei were detected by Hoechst (Invitrogen, H3570, 1:10000).

Quantitative Real-Time Reverse Transcriptase Polymerase Chain Reaction (qRT-PCR). Total RNA was isolated from 1×10⁶ cells using RNeasy Mini Kit (Qiagen, Valencia, CA). After the removal of genomic DNA, RNA is reversely transcribed into cDNA using Moloney murine leukemia virus RT (Invitrogen, Carlsbad, CA) with oligo (dT) primers (Invitrogen). Quantitative PCR was performed using SYBR green master mix (applied biosystem) in triplicates on a Real-Time PCR 7500 SDS system and software following manufacturer's instruction (Life Technologies, Grand Island, NY, USA). RNA content was normalized based on amplification of 18S ribosomal RNA (rRNA) (18S). Change folds=normalized data of experimental sample/normalized data of control. The specific primer pairs used for PCR are listed in Table 1.

TABLE 1
Real-Time PCR Primers

| | Forward(F)/ | | |
|---|---|---|---|
| | Reverse(R) | | |
| Gene | Primer | Primer sequences | SEQ ID NO: |
| Podoplanin | F | ACCGTGCCAGTGTTGTTCTG | 143 |
| | R | AGCACCTGTGGTTGTTATTTTGT | 144 |
| PSGL-1 | F | GAAAGGGCTGATTGTGACCCC | 145 |
| | R | AGTAGTTCCGCACTGGGTACA | 146 |
| CD40 | F | TGTCATCTGTGAAAAGGTGGTC | 147 |
| | R | ACTGGAGCAGCGGTGTTATG | 148 |
| CD147 | F | GTGGCGTTGACATCGTTGG | 149 |
| | R | CTATGTACTTCGTATGCAGGTCG | 150 |
| ICAM | F | GTGATGCTCAGGTATCCATCCA | 151 |
| | R | CACAGTTCTCAAAGCACAGCG | 152 |
| Fc receptor | F | AGGGCCTCCATCTGGACTG | 153 |
| | R | GTGGTTCTGGTAATCATGCTCTG | 154 |
| LFA1 | F | CCAGACTTTTGCTACTGGGAC | 155 |
| | R | GCTTGTTCGGCAGTGATAGAG | 156 |
| VWF | F | CTCTTTGGGGACGACTTCATC | 157 |
| | R | TCCCGAGAATGGAGAAGGAAC | 158 |

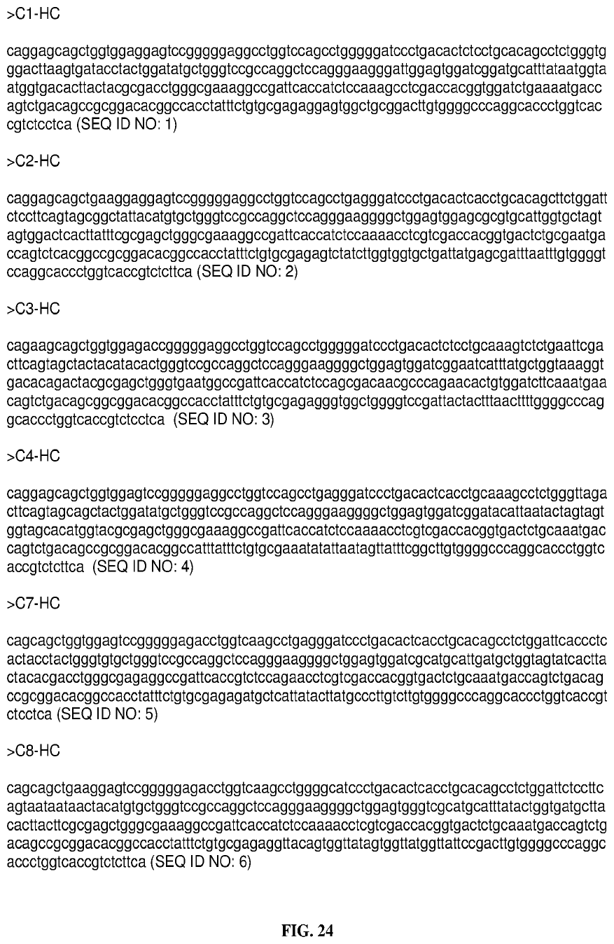

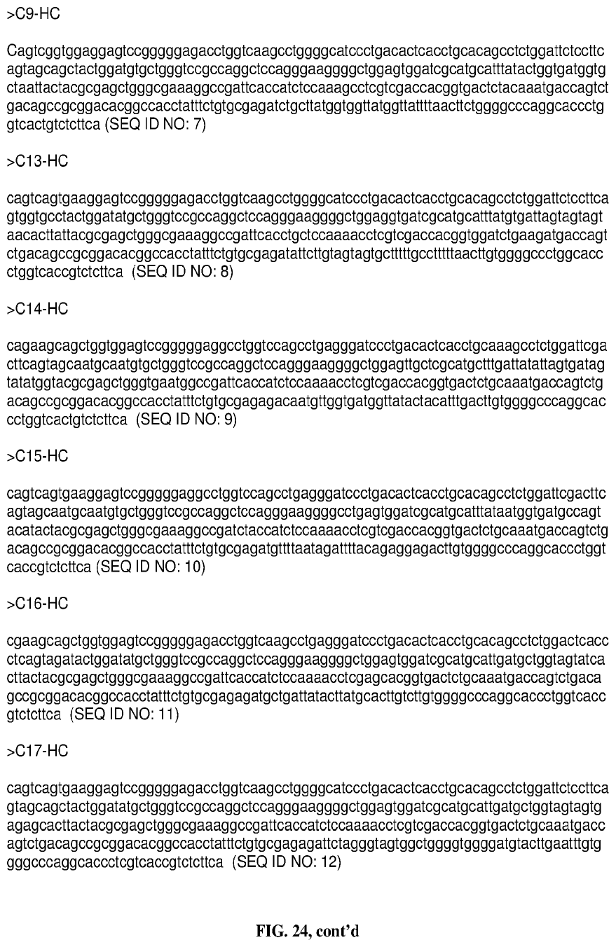

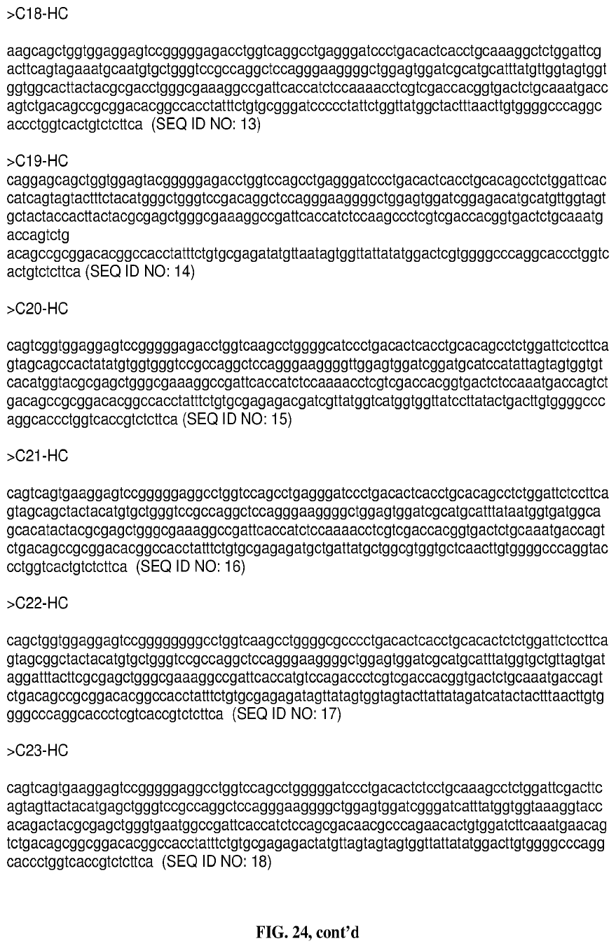

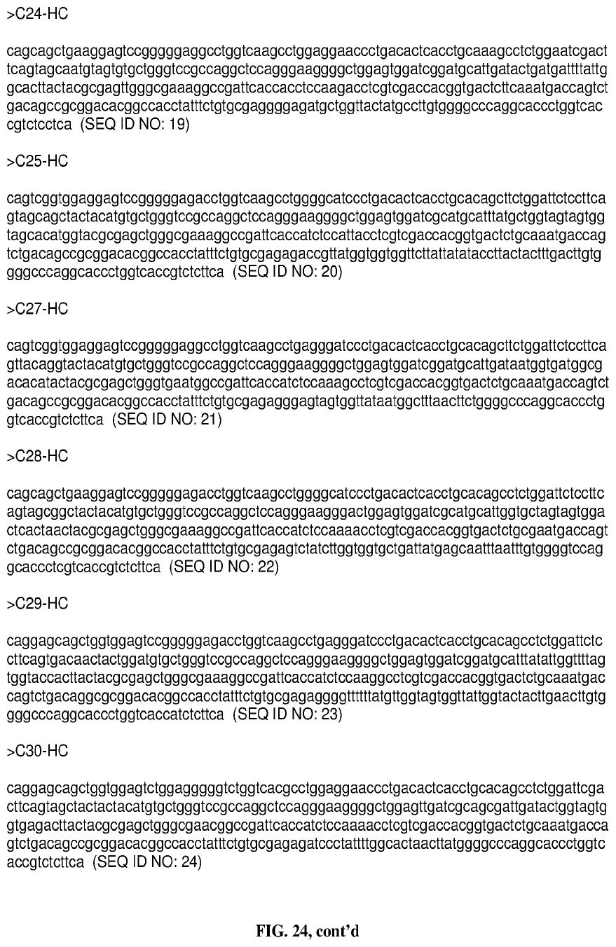

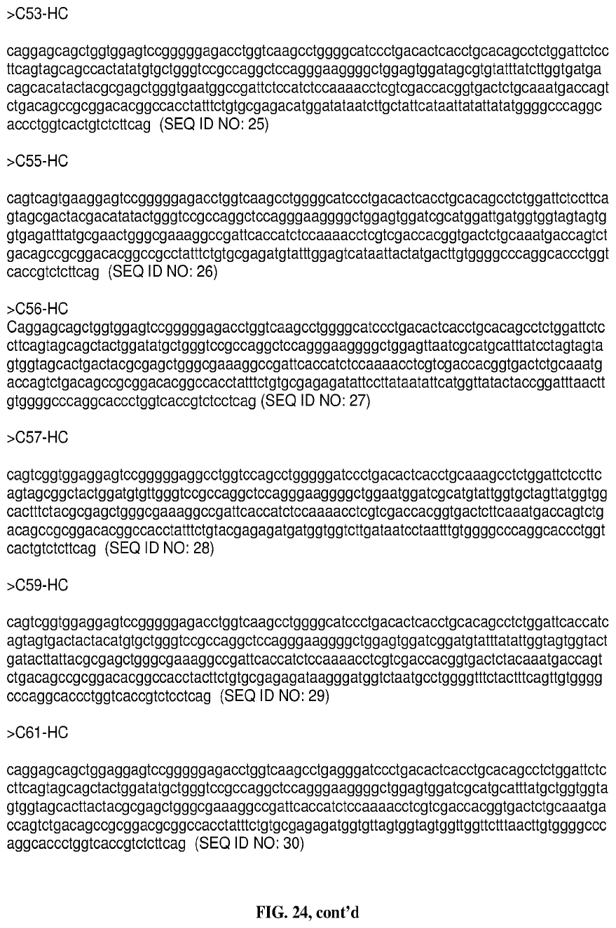

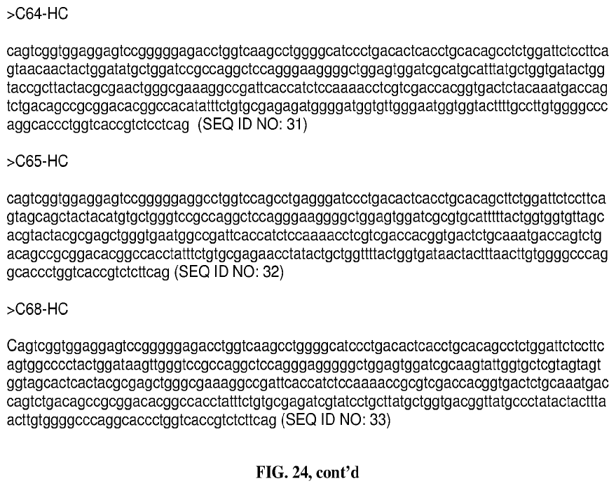

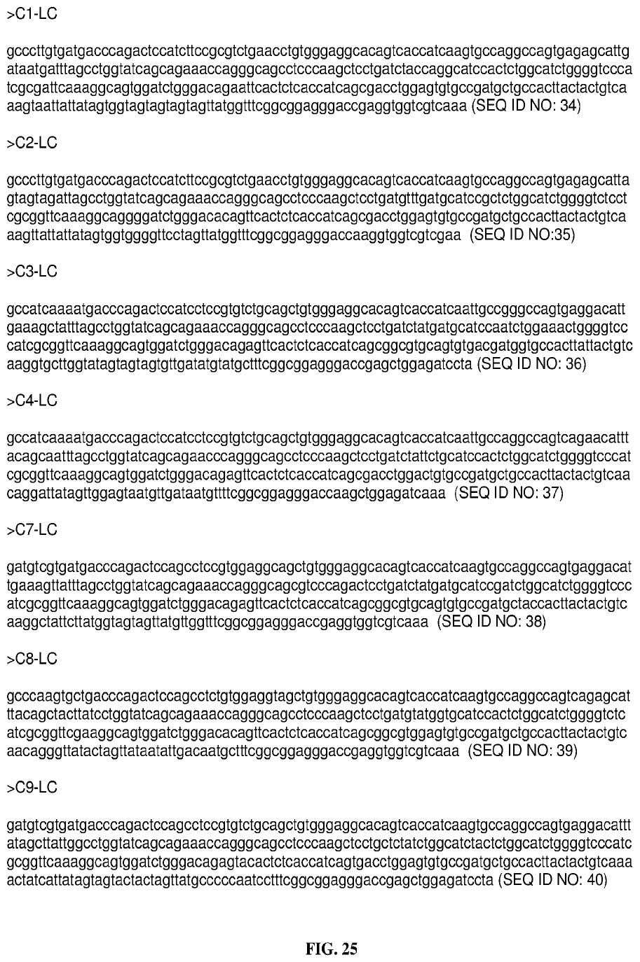

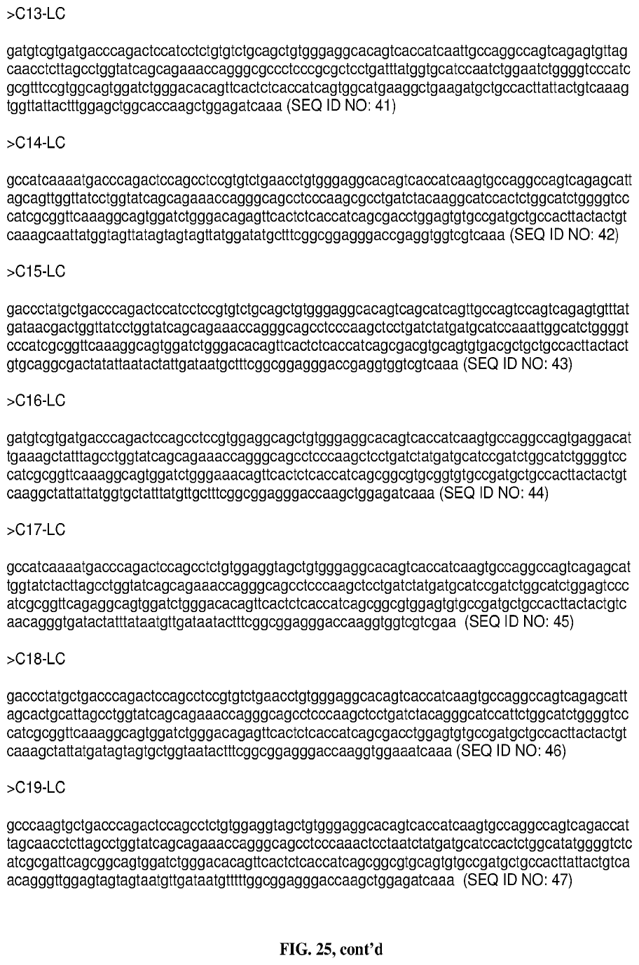

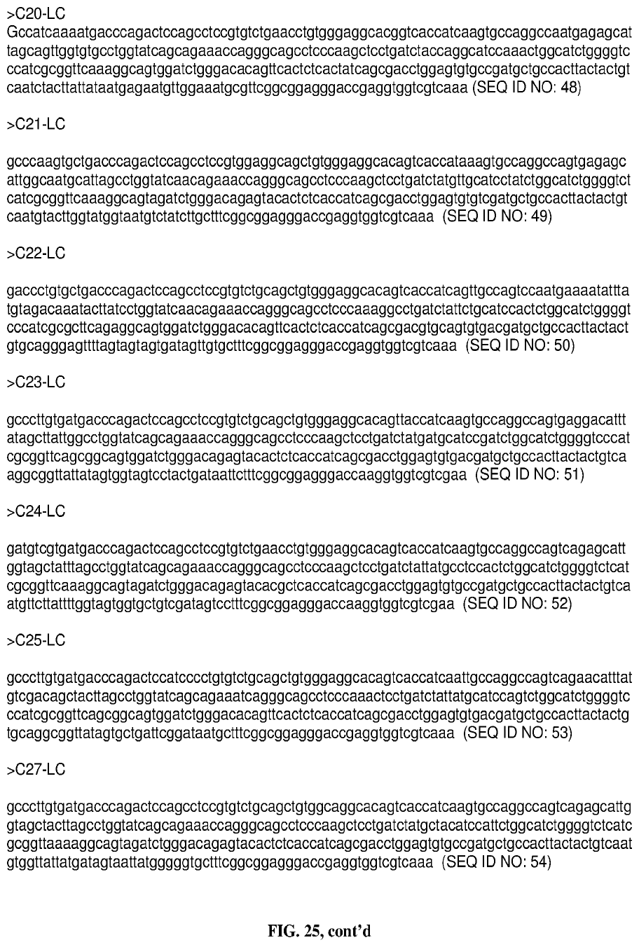

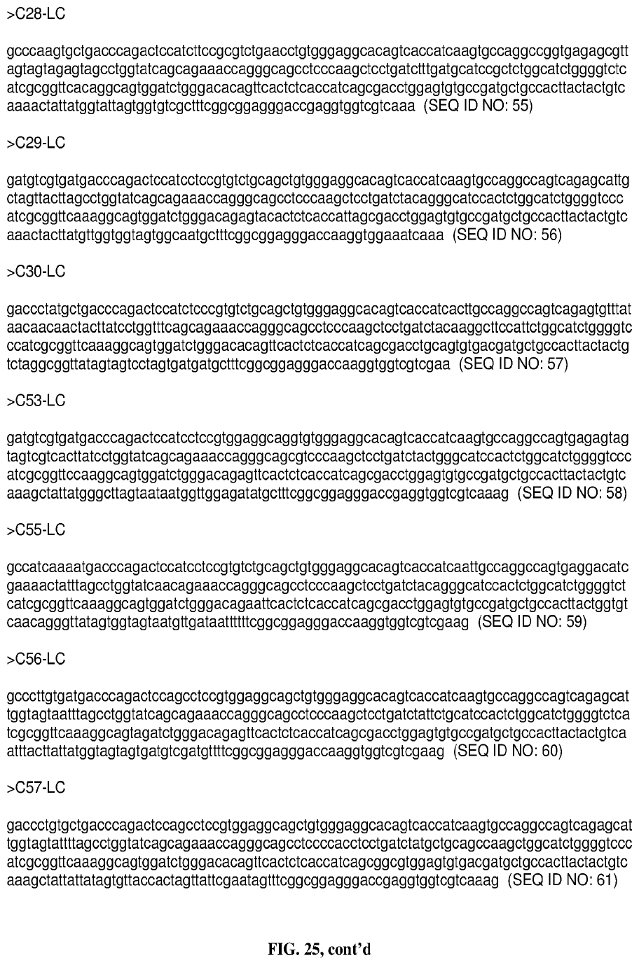

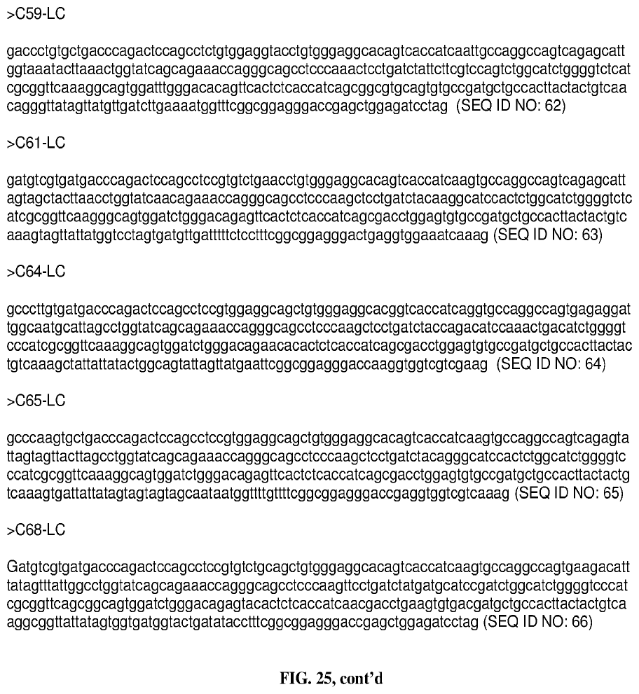

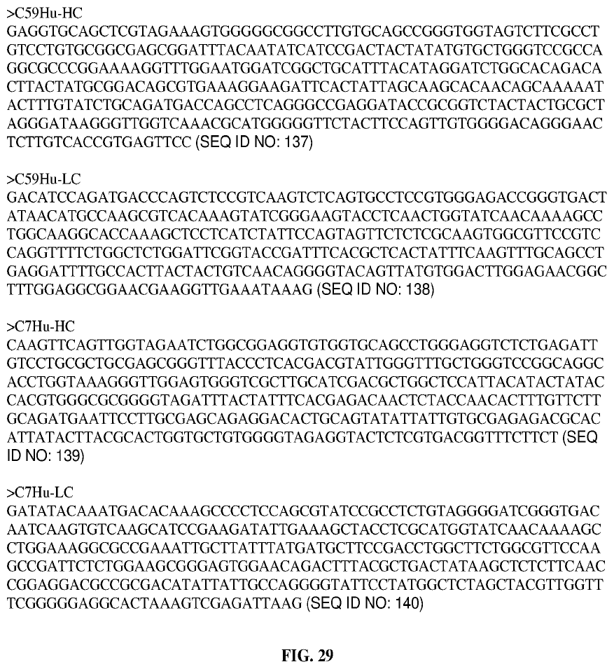

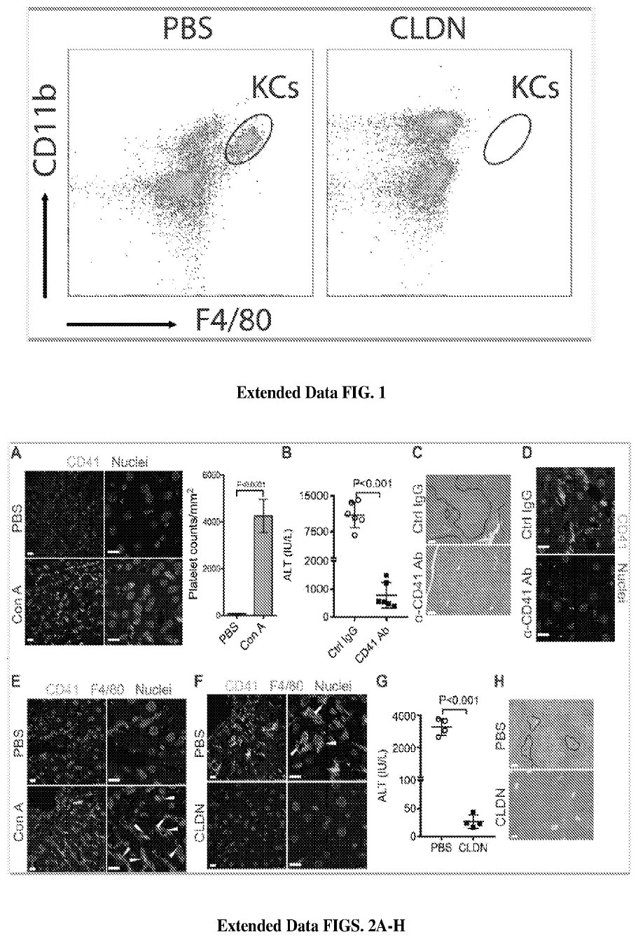

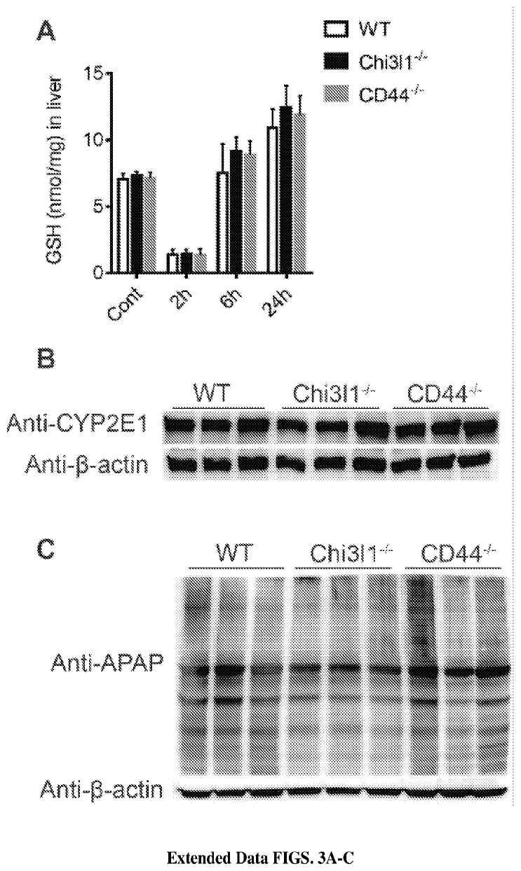

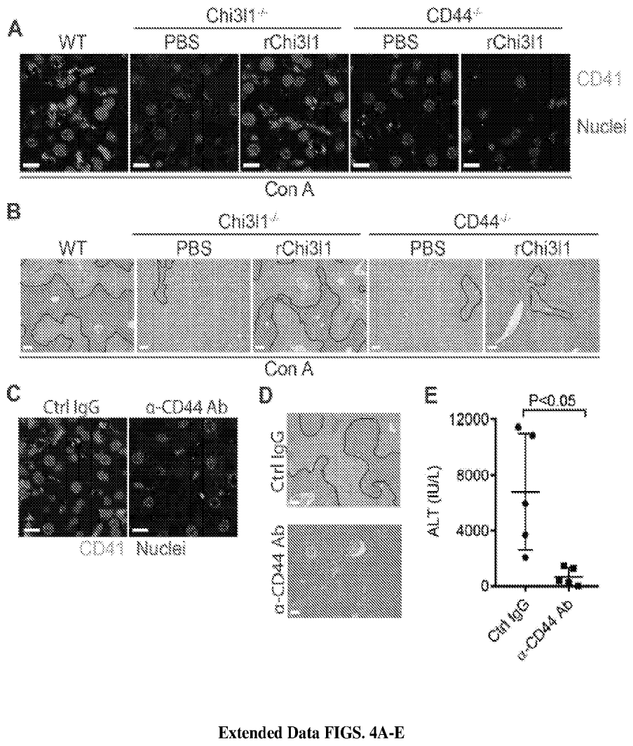

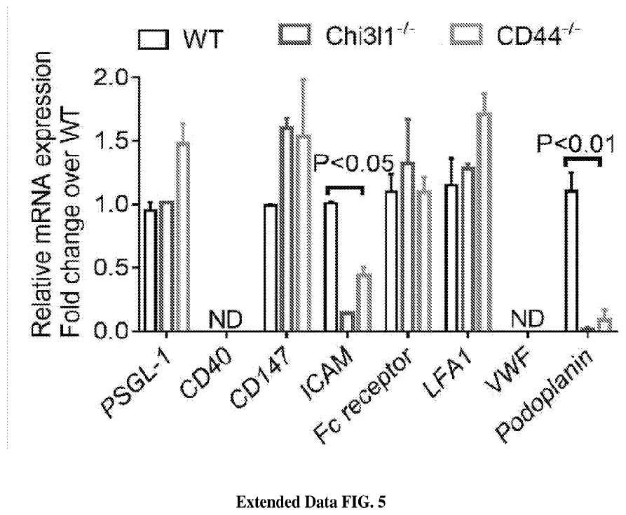

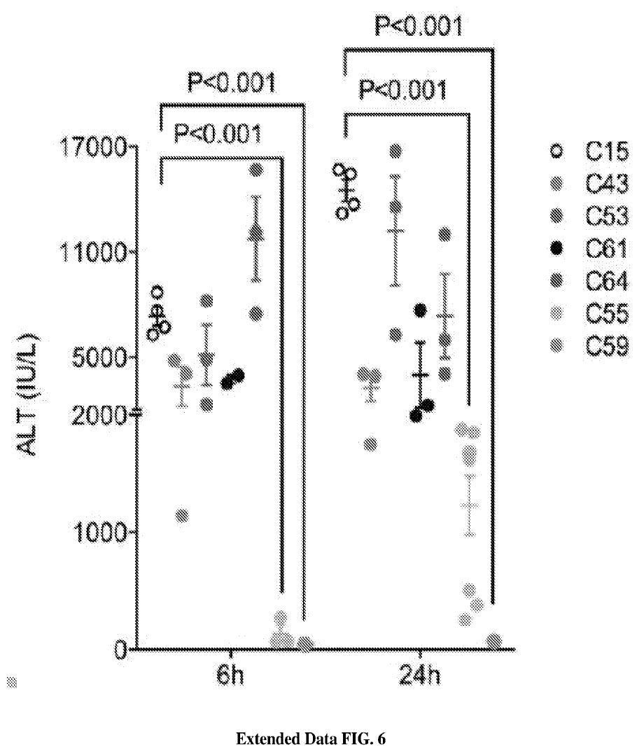

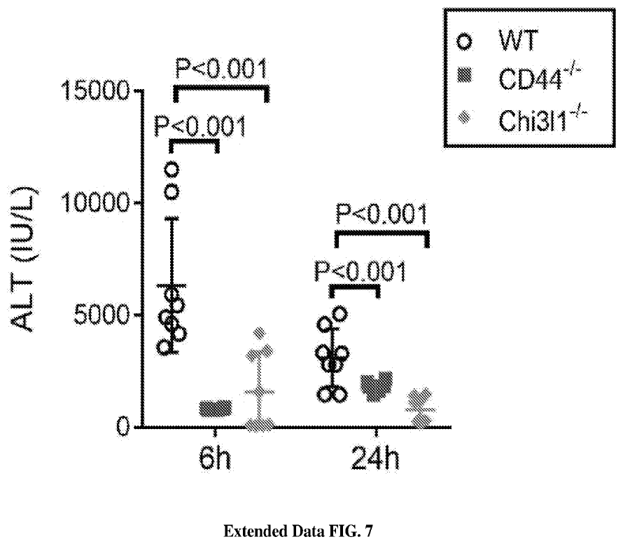

Generation of CHI3L1 mAbs. Monoclonal antibodies (mAbs) against CHI3L1 were generated by immunization of rabbits and isolation of antibodies from single B cells. Human CHI3L1 protein was used for antibody generation and was expressed in HEK293 cells. The protein has a 6×HIS-tag and avi-tag and the recombinantly expressed protein is purified to >95% purity using Ni-NTA resin. Rabbits (NZW, Charles River) were immunized with the recombinantly produced CHI3L1 using standard immunization procedures with 3 boost injections after primary priming immunization. The titer of anti-CHI3L1 sera was determined by series of dilutions of serum in ELISA for binding on CHI3L1 protein coated on 96-well plates (max-sorb plates, Nunc). When serum titer reached >10⁶ and peripheral blood samples were collected from the immunized rabbits for B cell isolation from the freshly prepared peripheral blood mononuclear cells (PBMCs). PBMCs were prepared using the IACCUSPIN™ System-Histopaque®-1077 kit (Sigma) and antigen specific memory B cells were enriched using Miltenyi Streptavidin MicroBeads isolation system. Single B cells were plated into 96-well cell culture plates and cultured with feeder cells and cytokine mixture in the culture medium with 10% FBS at 37° C. for 7-10 days in a cell culture incubator with 5% CO₂ and 95% humidity. The antibodies in the culture supernatants were assayed for CHI3L1 bindings. Cells from the positives wells were lysed, total RNA was isolated, and cDNA was synthesized using a superscript reverse transcriptase II (Invitrogen) according to manufacturer's suggestion. DNA sequences of antibody variable regions from both heavy chains and light chains were amplified by polymerase chain reaction (PCR) using a set of designed primers for antibody sequence amplification. Antibody variable fragments were cloned into an expression vector for production of each antibodies. Variable sequences of both DNA and amino acid sequences are listed in the FIGS. 24-29. CDRs of the anti-CHI3L1 monoclonal antibodies are identified using the IMGT program (world-wide web at IMGT.org) and are listed in Tables A & B.

Expression and purification of anti-CHI3L1 mAbs. Selected CHI3L1 binding hits were expressed as full length IgGs using a mammalian expression vector system in human embryonic kidney (HEK293) cells (Invitrogen). Antibodies were purified using protein A affinity resin by a fast protein liquid chromatography (FPLC). Purified CHI3L1 binding antibodies were characterized for their binding affinity and biological properties.

Binding affinity of anti-CHI3L1 monoclonal antibodies to the CHI3L1 protein using ELISA method. Binding of CHI3L1 by monoclonal antibodies was first screened by ELISA using supernatants collected from the B cell cultures and cross reactivity to mouse CHI3L1 protein was determined (FIG. 7).

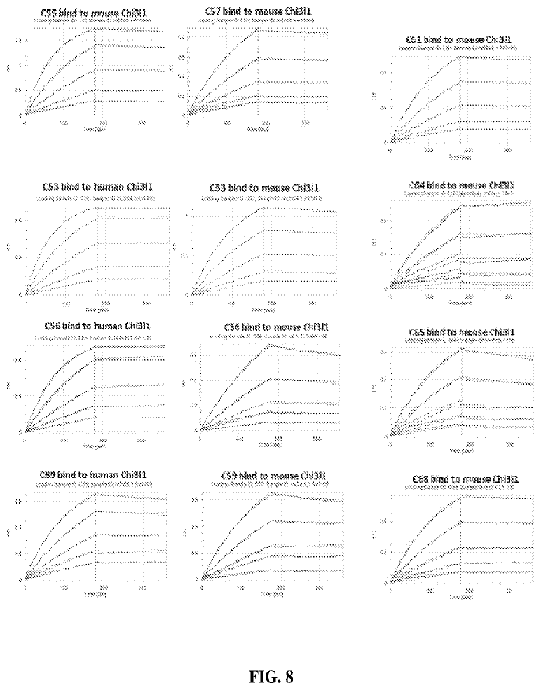

Binding affinity of anti-CHI3L1 monoclonal antibodies determined using Bio-layer interferometry (BLI), a sensor-based Octet instrument. For antibody affinity measurement, antibody (30 μg/mL) was loaded onto the protein A biosensors for 4 min. Following by kinetics buffer washing to baseline, the loaded biosensors were exposed to a series of recombinant CHI3L1 protein at 0.1-200 nM range and background subtraction was used to correct for any background from sensor drifting. All experiments were performed with shaking at 1,000 rpm. Background wavelength shifts were measured from reference biosensors that were loaded with antibody and followed with buffer without antigen. Kinetic sensorgrams for each antibodies are shown in FIG. 8. ForteBio's data analysis software was used to fit the data to a 1:1 binding model to extract an association rate and dissociation rate. The KD was calculated using the ratio of koff/kon and the estimated values of KD for CHI3L1 mAbs in Table C.

Epitope binning and grouping of CHI3L1 mAbs. Pairwise binding competition among anti-CHI3L1 mAbs was used to determine the binding epitopes of each mAbs using Octet instrument and protein A biosensors. The epitope bins are summarized in Table D. Fragments of CHI3L1 proteins were used to group CHI3L1 antibodies and two groups is listed in Table E.

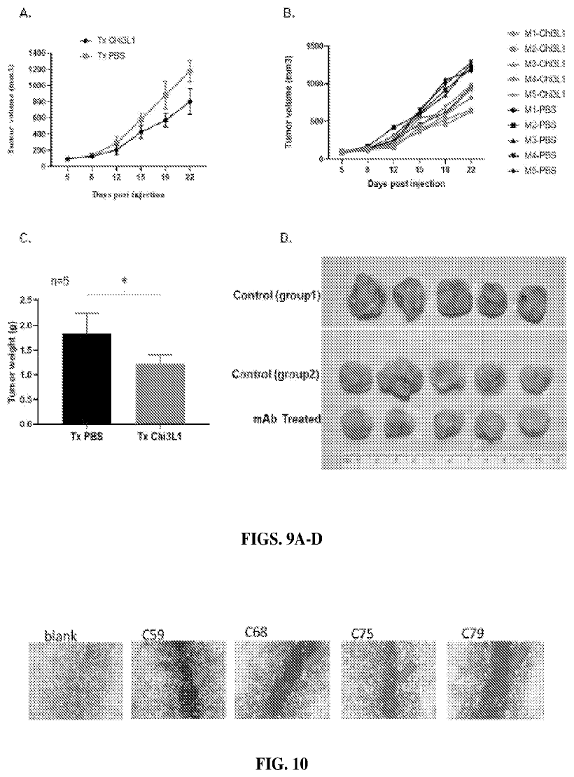

In vivo study of CHI3L1 antibody for inhibition of breast cancer tumor growth. Mouse breast tumor cells (4T1) were implanted at 1 million/mouse at mammary fat pads of 5-7 week old female mice. Mice were treated with CHI3L1 antibody C59 at 10 mg/kg once a week after 4 days of tumor cell implantation and establishing local tumors. Tumor size was monitored twice weekly until study termination when tumor sizes in control group reach to close to 10% of body weight. CHI3L1 mAb C59 that recognize mouse CHI3L1 showed significant inhibition of tumor growth as shown in both tumor growth curves and tumor weight in the end of the study (FIGS. 9A-D).

CHI3L1 antibody inhibited cancer cell (EMT6) migration in vitro. Cancer cells were cultured for 24 hours and scratch gaps were made on adherent cells using a sterile tip. Chil3L1 mAb were added at 10 μg/ml and treated in the cell cultures for 48 hours before imaging (FIG. 10).

Statistical analyses. Data were presented as mean±SEM. Statistical analyses were carried out using GraphPad Prism (GraphPad Software). Comparisons between two groups were carried out using unpaired Student t test. Comparisons among multiple groups (n>=3) were carried out using one-way ANOVA. P values are as labeled and less than 0.05 was considered significant. Platelets counts/mm², Platelets aggregates area, Platelet adherence to KCs or LSECs, KCs size were analyzed by Image J software.

### Example 2—Results and Discussion

Hepatic accumulation of platelets and their contribution to acute liver injury. Thrombocytopenia is often observed in patients with APAP overdose.^{2,4-6} The inventors hypothesized that the platelets may be recruited to the liver. They performed immunohistochemical (IHC) staining of liver biopsies from patients with APAP-induced liver injury (AILI) and detected markedly increased numbers of platelets compared with normal liver biopsies (FIG. 1A). Similarly, substantial accumulation of platelets in the liver was observed in mice treated with APAP (FIG. 1B). Curiously, the platelets accumulate in areas around the central veins where injury ensues. It is reported that depletion of platelets prior to APAP treatment can prevent liver injury in mice.⁷ These data demonstrated that even after APAP treatment, depletion of platelets could still attenuate AILI (FIGS. 1C-E). These data also indicate a critical contribution of platelets to AILI. An important question is how do platelets accumulate in the liver?

Kupffer cells (KCs) promote hepatic platelet accumulation. Two recent studies demonstrated that Kupffer cells mediate platelet recruitment into the liver during bacterial infection and nonalcoholic fatty liver disease.12,13 To examine if KCs have a similar effect in AILI, the inventors stained liver biopsies from patients with AILI using anti-CD68 and anti-CD41 antibodies. The data showed that the platelets were located either directly on or in the vicinity of the macrophages (FIG. 2A). In the livers of APAP-treated mice, IHC staining for KCs (anti-F4/80 antibody) and platelets (anti-CD41 antibody) also showed attachment of platelets to KCs (FIG. 2B). Quantification of the staining confirmed that there were higher numbers of platelets adherent to KCs than to LSECs (FIG. 2C). These observations led to the hypothesis that KCs might play an essential role in the recruitment of platelets into the liver during injury. To test this hypothesis, the inventors depleted KCs using clodronate encapsulated in liposomes (CLDN) at 9 h prior to APAP treatment and measured KCs and platelets in the liver at 6 h post-APAP challenge. They confirmed that KCs were nearly completely depleted (Extended Data FIG. 1). Compared to control mice treated with empty liposomes, hepatic platelet accumulation was abrogated (FIG. 2D) and liver injury was significantly reduced in CLDN-treated mice (FIGS. 2E-F). These data suggest that KCs play a crucial role in the platelet recruitment into the liver, thereby contributing to the early phase of AILI.

To substantiate the above findings in another model of acute liver injury, the inventors performed similar experiments in mice treated with concanavalin A (Con A). Con A-induced liver injury (CILI) simulates immune-mediated acute hepatitis.³⁰ They observed a significant increase of the number of intrahepatic platelets in mice treated with Con A for 6 h, compared to PBS-treated control mice (Extended Data FIG. 2A). Platelet depletion by an anti-CD41 antibody markedly attenuated CILI (Extended Data FIGS. 2B-D). IHC co-staining for F4/80 and CD41 showed platelet adhesion to KCs (Extended Data FIG. 2E). Furthermore, KCs-depletion by CLDN abrogated platelet accumulation in the liver after Con A treatment and significantly reduced CILI (Extended Data FIGS. 2F-H). These data revealed that the KCs-mediated hepatic platelet accumulation and the pathological role of platelets may be a common phenomenon during acute liver injury.

Identification of CHI3L1 and its receptor as potential activator of KCs. A natural next question is how KC are regulated to promote platelet recruitment. It is not likely that APAP can directly modulate KCs because APAP toxicity is caused by the reactive metabolite, N-acetyl-para-quinone imine (NAPQI). The inventors hypothesized that KCs are regulated by a soluble factor released rapidly upon liver injury. To identify this factor, the inventors utilized two unbiased approaches. First, they compared global gene expression profiles of liver tissues from APAP- vs. PBS-treated mice. Second, they measured protein expression levels of a panel of soluble factors known to modulate macrophage activation.^{18,31} Both sets of data showed a dramatic increase of Chi31 after APAP challenge (FIG. 2H). To investigate the involvement of CHI3L1 in activating KCs to promote platelet recruitment, the inventors first examined the effect of CHI3L1 on hepatic platelet accumulation. Compared with WT mice, the CHI3L1^{-/-} mice had a dramatically reduced number of platelets in the liver after APAP treatment. Administration of mouse recombinant CHI3L1 protein (rCHI3L1) to the CHI3L1^{-/-} mice restored the platelet accumulation in the liver (FIG. 2I). These data suggest that CHI3L1 is critical in facilitating platelet accumulation in the liver.

CHI3L1 interacts with CD44 on KCs. To further understand how CHI3L1 mediates the function of KCs to recruit platelets, the inventors set out to identify its receptor on KCs. They isolated KCs from WT mice treated with APAP for 3 h and incubated the cells with His-tagged rCHI3L1. The cell lysate was subjected to immunoprecipitation using an anti-His antibody. The “pulled down” fraction was subjected to LC/MS analyses. A partial list of proteins identified, including CHI3L1 itself, is shown in (Extended Data Table 1). Because CD44 is a cell membrane protein, the inventors decided to investigate it is a receptor for CHI3L1. They performed/immunoprecipitation experiments using liver homogenates obtained from WT and CD44^{-/-} mice after APAP treatment. As shown in FIG. 3A, the anti-CD44 antibody could “pull down” CHI3L1 from WT but not CD44^{-/-} liver homogenates. Interferometry measurements using recombinant human CHI3L1 (rhCHI3L1) revealed a high binding affinity between CHI3L1 and CD44 (Kd=251 nM; FIG. 3B). Moreover, the inventors incubated rhCHI3L1 with human CD44 and then immunoprecipitated with an anti-CD44 antibody. The data confirmed a direct binding between CHI3L1 and CD44 (FIG. 3C). Consistent with the notion that many cell types express CD44, they detected CD44 express on various hepatic non-parenchymal cells (NPCs) except B cells and DCs (summarized in Extended Data Table 2). However, when they incubated liver NPCs with His-tagged rCHI3L1, the inventors observed that the only CD44-expressing cells that could bind to CHI3L1 were KCs (FIG. 3D). Together, these results suggest that CD44 is a receptor for CHI3L1 on KCs.

CHI3L1 promotes platelets adhesion to KCs through CD44. To investigate the involvement of CHI3L1 and CD44 in hepatic platelet recruitment and liver injury, the inventors treated WT, CHI3L1^{-/-} and CD44^{-/-} mice with APAP. In contrast to the large number of platelets accumulated in the liver of WT mice, very few platelets could be found in the livers of CHI3L1^{-/-} and CD44^{-/-} mice (FIG. 4A). Injection of rCHI3L1 to the CHI3L1^{-/-}, but not CD44^{-/-} mice, restored platelet accumulation in the liver and enhanced liver injury toward the level observed in WT mice (FIGS. 4A-C). Most importantly, the majority of the platelets by CHI3L1/CD44 axis appear to adhere to KCs. Moreover, blocking CD44 by an antibody in CHI3L1^{-/-} mice abrogated the effects of rCHI3L1 in promoting KCs to recruit platelets and thus far less liver injury (FIGS. 4D-F). Together, these data demonstrate that CD44 is required for the function of CHI3L1 in promoting hepatic platelets accumulation and AILI.

Cytochrome P450 2E1 (CYP2E1)-mediated APAP bio-activation to NAPQI and the detoxification of NAPQI by glutathione (GSH) are important in determining the degrees of AILI.³²⁻³⁸ Thus, although unexpected, the inventors examined the possibility that deletion of CHI3L1 or CD44 might affect APAP bio-activation. They did not observe any differences in the levels of GSH, liver CYP2E1 protein expression, or NAPQI-protein adducts among WT, CHI3L1^{-/-} and CD44^{-/-} mice (Extended Data FIGS. 3A-C). These data indicate that CHI3L1 and CD44 deletion do not affect APAP bio-activation and its direct toxicity to hepatocytes.

Furthermore, the inventors examined the role of CHI3L1/CD44 axis in promoting platelet recruitment and liver injury in the CILI model. They found that rCHI3L1 could restore hepatic platelet accumulation and enhance liver injury in CHI3L1^{-/-} mice, but not in CD44^{-/-} mice (Extended Data FIGS. 4A-B). Moreover, the effect of rCHI3L1 on CHI3L1^{-/-} mice was abrogated by blocking CD44 using an antibody (Extended Data FIGS. 4C-E). These data indicate that the role of CHI3L1/CD44 axis in hepatic platelet accumulation and liver injury is not limited to AILI.

CHI3L1/CD44 signaling in KCs upregulates podoplanin expression and platelet adhesion. To investigate how CHI3L1/CD44 signaling in KCs promotes platelet recruitment, the inventors measured KC-expression of a panel of adhesion molecules important in platelet recruitment.³⁹⁻⁴⁴ They found that podoplanin is expressed at a much higher level in KCs from WT mice than those from CHI3L1^{-/-} or CD44^{-/-} mice (Extended Data FIG. 5). To compare cellular expression of podoplanin, the inventors isolated KCs, LSECs, hepatic stellate cells (HSC) and hepatocytes from naïve and APAP-treated WT mice. As shown in FIG. 5A, the mRNA level of podoplanin was up-regulated after APAP treatment, but only in KCs, not in other major cell types of the liver. Moreover, the expression levels of podoplanin were dramatically lower in KCs from CHI3L1^{-/-} and CD44^{-/-} mice than those from WT mice. Interestingly, rCHI3L1 treatment to CHI3L1^{-/-}, but not CD44^{-/-} mice, could markedly increase the levels of podoplanin mRNA and protein expressions in KCs (FIGS. 5B-D).

To further examine the role of podoplanin in mediating platelet adhesion to KCs, the inventors blocked podoplanin using an anti-podoplanin antibody in CHI3L1^{-/-} mice reconstituted with rCHI3L1. As shown in FIGS. 5E-G, blockade of podoplanin not only abrogated rCHI3L1-mediated platelet recruitment into the liver, but also significantly reduced its effect on increasing AILI in CHI3L1^{-/-} mice. C-type lectin-like receptor 2 (Clec-2) is the only receptor on platelets that has been reported to bind to podoplanin⁴³. To further elucidate the role of podoplanin in mediating KCs/platelet adhesion, the inventors isolated KCs from WT mice treated with APAP. After treating the KCs with anti-podoplanin antibody or IgG as control, they added platelets. Immunofluorescence staining of podoplanin and Clec-2 showed that the Clec-2 expressing platelets only bound to IgG-treated, but not anti-podoplanin-treated KCs (FIG. 5H). Together, these data demonstrate that KCs recruit platelets through podoplanin and Clec-2 interaction, and that the podoplanin expression on KCs is regulated by CHI3L1/CD44 signaling.

Evaluation of the therapeutic potential of targeting CHI3L1 in the treatment of AILI. AILI is a serious medical problem, which lacks effective therapies. While elucidating the underlining biology of CHI3L1 in AILI, the inventors also generated monoclonal antibodies specifically recognizing either the mouse or human CHI3L1. First, they screened a panel of anti-mouse CHI3L1 antibodies (α-mCHI3L1 Ab) for their efficacies of attenuating AILI. After 3 h of APAP challenge, the inventors injected WT mice with a panel of α-mCHI3L1 Abs or IgG as control. They found that clones 55 and 59 (C55 and C59) could markedly reduce ALT levels at 6 h and 24 h post-APAP (Extended Data FIG. 6). They also performed additional experiments using C59 and the data showed that C59 could effectively inhibit hepatic platelet accumulation (FIG. 6A) and attenuate APAP-induced hepatocyte necrosis (FIG. 6B).

Interestingly, in both serum samples and liver biopsies from patients with AILI, the protein expression levels of CHI3L1 were dramatically increased compared to those from normal individuals (FIGS. 6D-E). Because the amino acid sequence homology between human and mouse CHI3L1 is quite high (76%), the inventors treated CHI3L1^{-/-} mice with recombinant human CHI3L1 (rhCHI3L1) in order to evaluate the effects of the anti-human CHI3L1 antibodies (α-hCHI3L1 Ab) the inventors generated. The data showed that rhCHI3L1, similar to mouse CHI3L1, could promote hepatic platelets recruitment and increase AILI in the CHI3L1^{-/-} mice (FIGS. 6F-H). They screened all α-hCHI3L1 Abs generated via immunohistochemistry staining on liver biopsies from patients with AILI and picked the clone worked best for in vivo studies. To the inventors' excitement, the α-hCHI3L1 Ab treatment could abrogate platelet recruitment and dramatically reduce liver injury (FIGS. 6F-H). Together, these data indicate that monoclonal antibody-based blocking of CHI3L1 may be an effective therapeutic strategy to treat AILI, and potentially other acute liver injuries.

Monoclonal antibodies targeting chitinase 3-like-1 (CHI3L1) that can be used to treat overdose acetaminophen (APAP)-induced liver failure and hepatocellular carcinoma (HCC). Acetaminophen (APAP) overdose results in death or liver transplantation in more than one-third of patients. As of 2018, the most common cause of acute liver failure in the United States remains to be overdose APAP-induced liver injury (AILI). It is estimated that 60 million Americans take APAP-containing products weekly and approximately 30,000 patients are admitted to intensive care units every year due to AILI. The direct cost of AILI is as high as $87 million annually. The effectiveness of the only antidote, N-acetylcysteine (NAC), declines rapidly after APAP ingestion, long before patients are admitted to the clinic with symptoms of severe liver injury. Therefore, there is an unmet need for developing new life-saving treatment to attenuate AILI.

HCC is one of the most common cancers with more than half a million new cases occurring worldwide each year. According to the World Health Organization (WHO), more than 1 million patients are projected to die from liver cancer by 2030, making it the 6th leading cause of cancer-related death. The incidence of HCC has been rising in the United States in past several decades. At present, HCC affects approximately 33,000 American and causes 27,000 deaths each year. Treatment options for HCC are very limited. A multikinase inhibitor, sorafenib, is currently the only first-line chemotherapy. However, the majority of the patients present with advanced HCC that is refractory to chemotherapy. Recently, an immune check-point inhibitor (nivolumab) has been approved by the FDA as a second line treatment. Given the critical role of inflammation in the development of HCC, more immunotherapies targeting the tumor microenvironment will emerge as a successful treatment approach.

CHI3L1 (YKL-40 in humans) is a chitinase-like soluble protein without chitinase activities. It can be produced by multiple cell types, including macrophages, neutrophils, fibroblasts, synovial cells, endothelial cells, and tumor cells. CHI3L1 has been implicated in many biological processes including apoptosis, inflammation, oxidative stress, infection, and tumor metastasis. But its involvement in APAP-induced liver failure has not been reported previously. Although there are clinical reports describing an association between HCC and elevated CHI3L1 expression, the role of CHI3L1 in HCC has not been previously reported. Targeting CHI3L1 to delay/treat HCC progression has also not been previously reported.

Anti-CHI3L1 antibodies markedly attenuate APAP-induced liver injury (AILI). Using a mouse model of AILI, the inventors found that CHI3L1, signaling through its receptor CD44, critically contributes to AILI. These data demonstrated that CHI3L1^{-/-} and CD44^{-/-} mice were resistant to AILI. Administration of recombinant CHI3L1 to CHI3L1^{-/-} mice, but not CD44^{-/-} mice, could restore AILI toward the degrees seen in wild-type (WT) mice. The inventors generated monoclonal antibodies specifically recognizing either the mouse or human CHI3L1. They then evaluated the therapeutic potential of these antibodies. For the anti-mouse antibodies, the inventors injected the antibodies to WT mice followed by APAP challenge. For the anti-human antibodies, they injected the antibodies to CHI3L1^{-/-} mice reconstituted with recombinant human CHI3L1. After screening a panel of the antibodies, the inventors identified several clones that could profoundly attenuate AILI. A manuscript describing these results is attached.

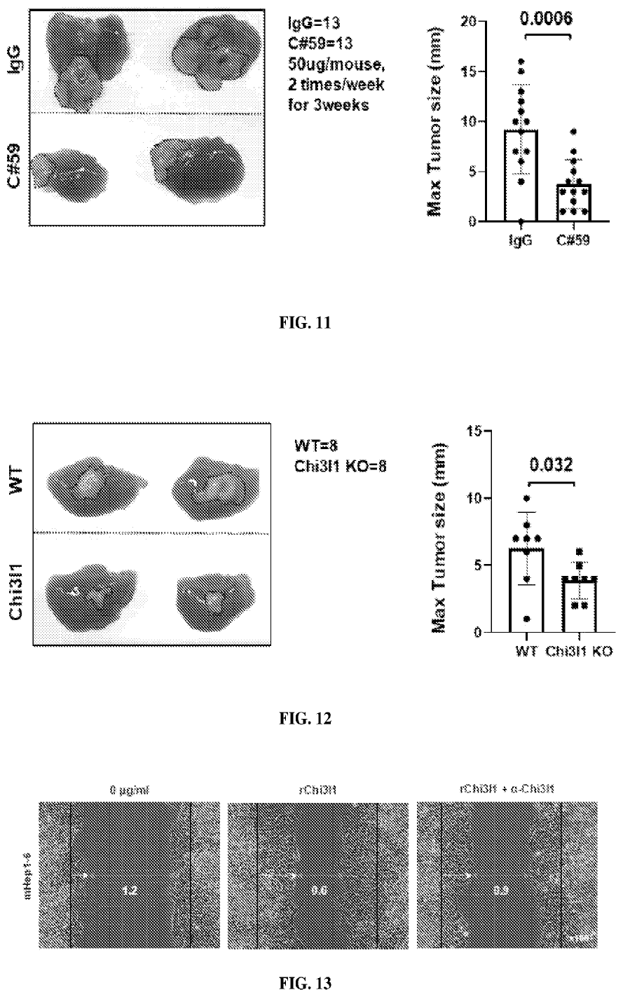

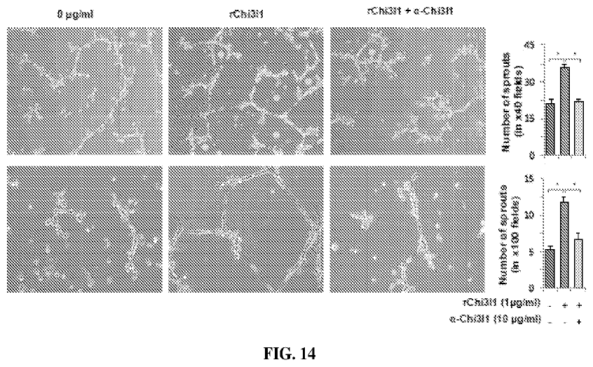

Anti-CHI3L1 antibodies significantly reduce HCC development. The inventors used a hepatic orthotopic murine model of HCC by injecting 2 million mouse HCC cell line (mHepa1-6) to the liver of WT C57B/6J mice. After one week, the mice were divided into two groups. One group was treated with an anti-mouse CHI3L1 antibody (C #59, 50 μg) 2 times per week for 3 weeks. The other group was treated with rabbit IgG as control. The data showed a profound inhibitory effect of C #59 on tumor growth. Combined results from three independent experiments are shown in FIG. 11. Furthermore, the inventors injected mHepa1-6 cells to the liver of WT and CHI3L1^{-/-} mice. After 4 weeks, the tumor sizes were significantly smaller in CHI3L1^{-/-} mice than WT mice, FIG. 12. It is likely that CHI3L1 in the tumor microenvironment plays a role in promoting tumor growth.

Discussion. While not wishing to be bound by any particular mechanism, increasing evidence suggests that platelets are sequestered within sinusoids in injured liver.^{7,9-13} Beyond their central role in hemostasis and tissue regeneration, platelets have been suggested to contribute to the initiation of liver injury.^{7,13,45} Here, the inventors identified KCs as a key player in intrahepatic platelet recruitment during early stages of acute liver injury. These studies revealed a critical role of the CHI3L1/CD44 signaling in mediating this function of KCs. Importantly, the inventors found that neutralizing CHI3L1 with monoclonal antibodies could effectively inhibit hepatic platelet accumulation and mitigate liver injury caused by APAP, supporting the potential and feasibility of targeting CHI3L1 as a therapeutic strategy to treat AILI.

Recently a study was published describing CD44 as a CHI3L1 receptor in gastric cancer.⁴⁶ Several other receptors for CHI3L1, including IL-13Rα2, CRTH2, TMEM219, and galectin-3 have also been reported.⁴⁶⁻⁵⁰ The finding that CHI3L1 binds to multiple receptors suggests a diverse involvement of CHI3L1 under different disease context. The inventors' observation that CHI3L1 predominantly binds to KCs, but not other CD44-expressing cells in the liver, suggests two possibilities. First, CHI3L1 may bind to a specific isoform of CD44 that is uniquely expressed by KCs. Second, KCs, but not other hepatic CD44-expressing cells, express a co-receptor that facilitates CHI3L1 binding to CD44 on KCs. These possibilities warrant further investigation.

As shown in the examples, platelets adhere to KCs through interaction between Clec-2 on platelets and podoplanin on KCs. In agreement with this finding, it has been reported that KCs recruit platelets via podoplanin/Clec-2 interaction during systemic S. typhimurium infection.⁵¹ Nonetheless, other pairs of receptor/ligand interactions have also been described to mediate platelet/KC adhesion. For example, Bacillus cereus- and Methicillin-resistant Staphylococcus aureus (MRSA) bacterial infection triggers sustained platelet adhesion to KCs through the interaction between glycoprotein IIb (GPIIb) and von Willebrand Factor (vWF).¹² A study of NASH reported that the hepatic recruitment of platelets was dependent on the interaction between GPIb on platelets and mucosal vascular addressin cell adhesion molecule 1 (MAdCAM-1) on KCs, whereas podoplanin, Clec-2 or vWF was not involved.¹³ The involvement of KCs in AILI has been a topic of debate, as KC depletion studies in mice have yielded opposing results.⁵²⁻⁵⁴ The current study suggests that the timing of CLDN treatment may be the key to reconcile the discrepancies. In the majority of the previous studies, mice were treated with APAP at 48 h after CLDN. Although KCs are depleted, there are abundant infiltrating macrophages at this time point. Thus, the phenotype observed in these studies reflects the function of the infiltrating macrophages, not just a result of KC depletion. Therefore, to avoid the confounding factor of infiltrating macrophages, the inventors chose a time point at which KCs were depleted but infiltrating macrophages were absent in the current studies to examine the role of KCs in platelet recruitment and AILI.

As demonstrated herein a previously unrecognized involvement of the CHI3L1/CD44 axis in AILI and provided insights into the mechanism by which CHI3L1/CD44 signaling promotes hepatic platelet accumulation and liver injury after APAP challenge. To take the inventors' findings one step further toward the clinic, they generated mouse and human anti-CHI3L1 monoclonal antibodies. These data demonstrated the feasibility of targeting CHI3L1 by a neutralizing antibody to attenuate AILI. Together, these studies provided strong evidence for the potential of targeting CHI3L1 as a novel therapeutic strategy to improve the clinical outcomes of AILI and perhaps other acute liver injury conditions.

Extended Data Table 1

| | | Score | Mass | Matches | | Sequence | | emPAI |
|---|---|---|---|---|---|---|---|---|
| 1 | sp\|Q8C196\|CPSM_MOUSE | 26134 | 165711 | 1380 | (123 [symbol] | 111 | (103) | 25.51 |
| 2 | sp\|O35490\|BHMT1_MOUSE | 3803 | 45448 | 191 | (167) | 24 | (19) | 7.21 |
| 3 | sp\|Q61362\|CH3L1_MOUSE | 3419 | 44150 | 211 | (187) | 25 | (25) | 9.79 |
| 4 | sp\|Q8BWT1\|THIM_MOUSE | 2532 | 42260 | 111 | (107) | 26 | (26) | 26.4 |
| 5 | sp\|P54869\|HMCS2_MOUSE | 2127 | 57300 | 107 | (92) | 27 | (25) | 6.47 |
| 6 | sp\|P56480\|ATPB_MOUSE | 1942 | 56265 | 101 | (89) | 27 | (26) | 8.71 |
| 7 | sp\|P63038\|CH60_MOUSE | 1782 | 61088 | 75 | (69) | 36 | (35) | 12.07 |
| 8 | sp\|Q03265\|ATPA_MOUSE | 1761 | 59830 | 86 | (79) | 33 | (31) | 7.97 |
| 9 | tr\|Q3UJ34\|Q3UJ34_MOUSE | 1725 | 46840 | 72 | (66) | 25 | (23) | 8.45 |
| 10 | sp\|Q8R0Y6\|AL1L1_MOUSE | 1545 | 99502 | 77 | (68) | 49 | (42) | 4.57 |
| 11 | tr\|A2NHM3\|A2NHM3_MOUSE | 970 | 24435 | 46 | (43) | 12 | (12) | 6.79 |
| 12 | tr\|I6L9E2\|I6L9E2_MOUSE | 910 | 26130 | 46 | (42) | 11 | (11) | 5.09 |
| 13 | sp\|Q8BMS1\|ECHA_MOUSE | 1510 | 83302 | 52 | (50) | 23 | (21) | 2.07 |
| 14 | tr\|Q3T9S7\|Q3T9S7_MOUSE | 1396 | 130519 | 70 | (64) | 42 | (37) | 2.28 |
| 15 | tr\|A0A0A0MQF6\|A0A0A0MQF6_MOUSE | 1343 | 38914 | 53 | (51) | 11 | (11) | 2.13 |
| 16 | tr\|Q3TVM2\|Q3TVM2_MOUSE | 1328 | 57073 | 56 | (51) | 22 | (20) | 3.3 |
| 17 | sp\|Q9DBM2\|ECHP_MOUSE | 1208 | 78822 | 53 | (44) | 27 | (25) | 2.84 |
| 18 | tr\|Q8C6E3\|Q8C6E3_MOUSE | 1197 | 60083 | 55 | (49) | 28 | (23) | 3.95 |
| 19 | tr\|Q5FW97\|Q5FW97_MOUSE | 1191 | 47453 | 50 | (45) | 22 | (20) | 5.13 |
| 20 | sp\|Q9QXD6\|F16P1_MOUSE | 1185 | 37288 | 46 | (45) | 16 | (16) | 6.7 |
| 21 | tr\|Q3TF14\|Q3TF14_MOUSE | 1178 | 48170 | 63 | (54) | 25 | (22) | 5.82 |
| 22 | sp\|Q9DBT9\|M2GD_MOUSE | 1164 | 97422 | 54 | (54) | 32 | (32) | 2.51 |
| 23 | sp\|P19157\|GSTP1_MOUSE | 1160 | 23765 | 51 | (46) | 10 | (8) | 4.55 |
| 24 | tr\|Q3TJ66\|Q3TJ66_MOUSE | 1086 | 39952 | 66 | (58) | 24 | (23) | 10.77 |
| 25 | tr\|Q3TQD9\|Q3TQD9_MOUSE | 1001 | 100662 | 52 | (47) | 32 | (29) | 1.97 |
| 26 | sp\|P19096\|FAS_MOUSE | 999 | 274994 | 46 | (44) | 38 | (37) | 0.62 |
| 27 | sp\|Q9R0H0\|ACOX1_MOUSE | 971 | 75000 | 51 | (48) | 27 | (25) | 2.93 |
| 28 | tr\|Q3UEQ9\|Q3UEQ9_MOUSE | 968 | 103650 | 39 | (37) | 25 | (23) | 1.54 |
| 29 | sp\|P26039\|TLN1_MOUSE | 950 | 271820 | 38 | (37) | 37 | (36) | 0.55 |
| 30 | sp\|Q546G4\|Q546G4_MOUSE | 949 | 70700 | 42 | (37) | 26 | (24) | 2.73 |
| 31 | sp\|Q61176\|ARGI1_MOUSE | 939 | 34957 | 38 | (36) | 15 | (15) | 4.59 |
| 32 | tr\|Q3UA81\|Q3UA81_MOUSE | 911 | 50423 | 52 | (46) | 15 | (13) | 2.78 |
| 33 | sp\|P32020\|NLTP_MOUSE | 906 | 59715 | 41 | (36) | 23 | (20) | 3.03 |
| 34 | tr\|CD44\|CD44_MOUSE | 881 | 72614 | 52 | (52) | 3 | (3) | 0.19 |
| 35 | tr\|Q3UQ71\|Q3UQ71_MOUSE | 868 | 63073 | 40 | (39) | 20 | (20) | 2.94 |
| 36 | tr\|D3Z041\|D3Z041_MOUSE | 859 | 79011 | 43 | (34) | 26 | (21) | 1.88 |
| 37 | tr\|A0A0R41135\|A0A0R41135_MOUSE | 804 | 53165 | 33 | (30) | 19 | (18) | 2.75 |
| 38 | sp\|P26443\|DHE3_MOUSE | 804 | 61640 | 48 | (43) | 25 | (23) | 4.56 |

[symbol]

indicates data missing or illegible when filed

Extended Data Table 1. Representative list of proteins bound to CHI3L1 via MS analysis. KCs were isolated from WT mice treated with APAP for 2 h, and then incubate rCHI3L1 with KCs lysis overnight. Proteins bound to rCHI3L1 were immune-precipitated with an anti-His antibody and undergo protein ID mass spectrometry analysis.

Extended Data Table 2

| Liver Cells | Marker | CD44 expression |
|---|---|---|
| Hepatocytes | FSC^{hi}, SSC^{hi} | − |
| LSECs | CD45 [symbol] CD146⁺ | ++ |
| Kupffer Cells | CD45⁺ F4/80^{hi} | ++ |
| Neutrophils | CD45⁺CD11b^{hi}Ly6G⁺ | + |
| Monocytes | CD45⁺CD11b^{hi}Ly6C⁺ | + |
| Dendritic cells | CD45⁺CD11c⁺MHCHII⁺ | − |
| B cells | CD19⁺ | − |
| T cells | CD3⁺ | ++ |
| NK cells | CD3⁺NK1.1⁺ | ++ |
| Eosinophils | CD45⁺Siglec F⁺ | ++ |
| HSCs | V450^{hi} | ++ |

[symbol]

indicates data missing or illegible when filed

Extended Data Table 2. Summary of CD44 expression by various cells in the mouse liver. Mouse hepatocytes and NPCs were isolated from mouse treated with APAP for either 2 h and 24 h. Isolated liver cells were labeled with indicated cell surface markers to define each distinguished cell populations. Each cell population were gated on single live cell population. (n=3 mice/group).

TABLE A
Amino acid sequences for CDRs of CHI3L1 mAbs

| | Heavy Chain | | | | | | Light Chain | | | | |
|---|---|---|---|---|---|---|---|---|---|---|---|
| | | SEQ ID | | SEQ | | SEQ ID | | SEQ ID | | | SEQ ID |
| mAbs | CDR1 | NO: | CDR2 | ID NO: | CDR3 | NO: | CDR1 | NO: | CDR2 | CDR3 | NO: |
| C1 | GWDLS | 159 | CIYNG | 192 | ARGVAADL | 225 | ESIDN | 258 | QAS | QSNYYSG | 291 |
| | DTY | | NG | | | | D | | | SSSSYG | |
| C2 | GFSFSS | 160 | IGASS | 193 | ARVYLGG | 226 | ESISS | 259 | DAS | QSYYYSG | 292 |
| | GYY | | GL | | ADYERFNL | | R | | | GVPSYG | |
| C3 | EFDFSS | 161 | IYAGK | 194 | ARGWLGS | 227 | EDIES | 260 | DAS | QGAWYS | 293 |
| | | | GDT | | DYYFNF | | Y | | | SSVDMYA | |
| C4 | GLDFSS | 162 | INTSS | 195 | AKYINSYF | 228 | QNIYS | 261 | SAS | QQDYSW | 294 |
| | SYW | | GS | | GL | | N | | | SNVDNV | |
| C7 | GFTLTT | 163 | IDAGS | 196 | ARDAHYTY | 229 | EDIES | 262 | DAS | QGYSYGS | 295 |
| | YW | | IT | | ALVL | | Y | | | SYVG | |
| C8 | GFSFSN | 164 | IYTGD | 197 | ARGYSGY | 230 | QSIYS | 263 | GAS | QQGYTSY | 296 |
| | NNY | | AYT | | SGYGYSD | | Y | | | NIDNA | |
| | | | | | L | | | | | | |
| C9 | GFSFSS | 165 | IYTGD | 198 | ARSAYGG | 231 | EDIYS | 264 | LAS | QNYHYSS | 297 |
| | SYW | | GAN | | YGYFNF | | L | | | TTSYAPN | |
| | | | | | | | | | | P | |
| C13 | GFSFSG | 166 | IYVIS | 199 | ARYSCSSA | 232 | QSVS | 265 | GAS | QSGYY | 298 |
| | AYW | | SSNT | | FCLFNL | | NL | | | | |
| C14 | GFDFSS | 167 | FDYIS | 200 | ARDNVGD | 233 | QSISS | 266 | KAS | QSNYGSY | 299 |
| | NA | | DSI | | GYTTFDL | | W | | | SSSYGYA | |
| C15 | GFDFSS | 168 | TYNGD | 201 | ARCFNRFY | 234 | QSVY | 267 | DAS | AGDYINTI | 300 |
| | NA | | AST | | RGDL | | DNDW | | | DNA | |
| C16 | GLTLSR | 169 | IDAGS | 202 | ARDADYTY | 235 | EDIES | 268 | DAS | QGYYYGA | 301 |
| | YW | | IT | | ALVL | | Y | | | IYVA | |
| C17 | GFSFSS | 170 | IDAGS | 203 | ARDSRVV | 236 | QSIGI | 269 | DAS | QQGDTIY | 302 |
| | SYW | | SEST | | AGVGMYL | | Y | | | NVDNT | |
| | | | | | NL | | | | | | |
| C18 | GFDFSR | 171 | IYVGS | 204 | AGSPYSG | 237 | QSIST | 270 | RAS | QSYYDSS | 303 |
| | NA | | GGGT | | YGYFNL | | A | | | AGNT | |
| C19 | GFTISS | 172 | MHVG | 205 | ARYVNSG | 238 | QTISN | 271 | DAS | QQGWSS | 304 |
| | TFY | | SATTT | | YYMDS | | L | | | SNVDNV | |
| C20 | GFSFSS | 173 | IHISS | 206 | ARDDRYG | 239 | ESISS | 272 | QAS | QSTYYNE | 305 |
| | SHY | | GVT | | HGGYPYT | | W | | | NVGNA | |
| | | | | | DL | | | | | | |
| C21 | GFSFSS | 174 | IYNGD | 207 | ARDADYA | 240 | ESIGN | 273 | VAS | QCTWYG | 306 |
| | SYY | | GST | | GVVLNL | | A | | | NVYLA | |
| C22 | GFSFSS | 175 | IYGAV | 208 | ARDSYSG | 241 | ENIYV | 274 | SAS | AGSFSSS | 307 |
| | GYY | | SDRI | | STYYRSYY | | DKY | | | DSCA | |
| | | | | | FNL | | | | | | |
| C23 | GFDFSS | 176 | IYGGK | 209 | ARDYVSSS | 242 | EDIYS | 275 | DAS | QGGYYS | 308 |
| | | | GTT | | GYYMDL | | L | | | GSPTDNS | |
| C24 | GIDFSS | 177 | IDTDD | 210 | ARGDAGY | 243 | QSIGS | 276 | YAS | QCSYFGS | 309 |
| | NV | | FIGT | | YAL | | Y | | | GAVDSP | |
| C25 | GFSFSS | 178 | IYAGS | 211 | ARDRYGG | 244 | QNIYV | 277 | YAS | AGGYSAD | 310 |
| | SYY | | SGST | | GSYYIPYY | | DSY | | | SDNA | |
| | | | | | FDL | | | | | | |
| C27 | GFSFSY | 179 | IDNG | 212 | ARGSSGY | 245 | QSIGS | 278 | ATS | QCGYYDS | 311 |
| | RYY | | DGDT | | NGFNF | | Y | | | NYGGA | |
| C28 | GFSFSS | 180 | IGASS | 213 | ARVYLGG | 246 | ESVS | 279 | DAS | QNYYGIS | 312 |
| | GYY | | GLT | | ADYEQFNL | | SR | | | GVA | |
| C29 | GFSFSD | 181 | IYIGF | 214 | ARGVFYV | 247 | QSIAS | 280 | RAS | QTTYVGG | 313 |
| | NYW | | SGTT | | GSGYWYY | | Y | | | SGNA | |
| | | | | | LNL | | | | | | |
| C30 | GFDFSS | 182 | IDTGS | 215 | ARDPYFGT | 248 | QSVY | 281 | KAS | LGGYSSP | 314 |
| | YYY | | GET | | NL | | NNNY | | | SDDA | |
| C53 | GFSFSS | 183 | IYLGD | 216 | ARHGYNL | 249 | ESSS | 282 | WAS | QSYYGLS | 315 |
| | SHY | | DST | | AIHNYII | | RH | | | NNGWRY | |
| | | | | | | | | | | A | |
| C55 | GFSFSS | 184 | IDGG | 217 | ARCIWSHN | 250 | EDIEN | 283 | RAS | QQGYSG | 316 |
| | DYD | | SSGEI | | YYDL | | Y | | | SNVDNF | |
| C56 | GFSFSS | 185 | IYPSS | 218 | ARDIPYNIH | 251 | QSIGS | 284 | SAS | QFTYYGS | 317 |
| | SYW | | SGST | | GYTTGFNL | | N | | | SDVDV | |
| C57 | GFSFSS | 186 | IGASY | 219 | TRDDGGL | 252 | QSIGS | 285 | AAA | QSYYYSV | 318 |
| | GYW | | GGT | | DNPNL | | - | | | TTSYSNS | |
| C59 | GFTISS | 187 | IYIGS | 220 | ARDKGWS | 253 | QSIGK | 286 | SSS | QQGYSYV | 319 |
| | DYY | | GTDT | | NAWGFYF | | Y | | | DLENG | |
| | | | | | QL | | | | | | |
| C61 | GFSFSS | 188 | IYAGG | 221 | ARDGVSG | 254 | QSISS | 287 | KAS | QSSYYGP | 320 |
| | SYW | | SGST | | SGWFFNL | | Y | | | SDVDFSP | |
| C64 | GFSFSN | 189 | IYAGD | 222 | ARDGDGV | 255 | ERIGN | 288 | QTS | QSYYYTG | 321 |
| | NYW | | TGTA | | GNGGTFA | | A | | | SISYE | |
| | | | | | L | | | | | | |
| C65 | GFSFSS | 190 | IFTGG | 223 | ARTYTAGF | 256 | QSISS | 289 | RAS | QSDYYSS | 322 |
| | SYY | | VST | | TGDNYFNL | | Y | | | SSNNGFV | |
| C68 | GFSFSG | 191 | IGARS | 224 | ARSYPAYA | 257 | EDIYS | 290 | DAS | QGGYYS | 323 |
| | PYW | | SGST | | GDGYALY | | L | | | GDGTDIP | |
| | | | | | YFNL | | | | | | |

TABLE B
Nucleic acid sequences for CDRs of CHI3L1 mAbs

| | Heavy Chain | | | Light Chain | | |
|---|---|---|---|---|---|---|
| | (SEQ ID NO: ) | | | (SEQ ID NO: ) | | |
| mAbs | CDR1 | CDR2 | CDR3 | CDR1 | CDR2 | CDR3 |
| C1 | gggtgggacttaagtgat | atttataatggtaatg | gcgagaggagtggctgc | gagagcattgataatgat | caggcatcc | caaagtaattattatagtg |
| | acctactgg | gtgacact | ggacttg | | | gtagtagtagtagttatgg |
| | | | | | | t |
| | 324 | 325 | 326 | 327 | | 328 |
| C2 | ggattctccttcagtagcg | attggtgctagtagtg | gcgagagtctatcttggt | gagagcattagtagtaga | gatgcatcc | caaagttattattatagtg |
| | gctattac | gactcact | ggtgctgattatgagcg | | | gtggggttcctagttatgg |
| | | | atttaatttg | | | t |
| | 329 | 330 | 331 | 332 | | 333 |
| C3 | gaattcgacttcagtagct | atttatgctggtaaag | gcgagagggtggctgg | gaggacattgaaagcta | gatgcatcc | ggtgcttggtatagtagta |
| | actac | gtgacaca | ggtccgattactacttta | t | | gtgttgatatgtatgct |
| | | | acttt | | | |
| | 334 | 335 | 336 | 337 | - | 338 |
| C4 | gggttagacttcagtagca | attaatactagtagtg | gcgaaatatattaatag | cagaacatttacagcaa | tctgcatcc | caacaggattatagttgg |
| | gctactgg | gtagcaca | ttatttcggcttg | t | | agtaatgttgataatgtt |
| | 339 | 340 | 341 | 342 | - | 343 |
| C7 | ggattcaccctcactacct | attgatgctggtagtat | gcgagagatgctcatta | gaggacattgaaagtta | gatgcatcc | caaggctattcttatggta |
| | actgg | cact | tacttatgcccttgtcttg | t | | gtagttatgttggt |
| | 344 | 345 | 346 | 347 | | 348 |
| C8 | ggattctccttcagtaata | atttatactggtgatgc | gcgagaggttacagtgg | cagagcatttacagctac | ggtgcatcc | caacagggttatactagtt |
| | ataactac | ttacact | ttatagtggttatggttat | | | ataatattgacaatgct |
| | | | tccgacttg | | | |
| | 349 | 350 | 351 | 352 | | 353 |
| C9 | ggattctccttcagtagca | atttatactggtgatgg | gcgagatctgcttatggt | gaggacatttatagctta | ctggcatct | caaaactatcattatagta |
| | gctactgg | tgctaat | ggttatggttattttaact | | | gtactactagttatgcccc |
| | | | tc | | | caatcct |
| | 354 | 355 | 356 | 357 | - | 358 |
| C13 | ggattctccttcagtggtg | atttatgtgattagtag | gcgagatattcttgtagt | cagagtgttagcaacctc | ggtgcatcc | caaagtggttattac |
| | cctactgg | tagtaacact | agtgctttttgccttttta | | | |
| | | | acttg | | | |
| | 359 | 360 | 361 | 362 | - | 363 |
| C14 | ggattcgacttcagtagca | tttgattatattagtga | gcgagagacaatgttgg | cagagcattagcagttgg | aaggcatcc | caaagcaattatggtagtt |
| | atgca | tagtata | tgatggttatactacatt | | | atagtagtagttatggata |
| | | | tgacttg | | | tgct |
| | 364 | 365 | 366 | 367 | - | 368 |
| C15 | ggattcgacttcagtagca | atttataatggtgatgc | gcgagatgttttaatag | cagagtgtttatgataac | gatgcatcc | gcaggcgactatattaata |
| | atgca | cagtaca | attttacagaggagactt | gactgg | | ctattgataatgct |
| | | | g | | | |
| | 369 | 370 | 371 | 372 | - | 373 |
| C16 | ggactcaccctcagtagat | attgatgctggtagtat | gcgagagatgctgatta | gaggacattgaaagcta | gatgcatcc | caaggctattattatggtg |
| | actgg | cact | tacttatgcacttgtcttg | t | | ctatttatgttgct |
| | 374 | 375 | 376 | 377 | - | 378 |
| C17 | ggattctccttcagtagca | attgatgctggtagta | gcgagagattctagggt | cagagcattggtatctac | gatgcatcc | caacagggtgatactattt |
| | gctactgg | gtgagagcact | agtggctggggtgggga | | | ataatgttgataatact |
| | | | tgtacttgaatttg | | | |
| | 379 | 380 | 381 | 382 | - | 383 |
| C18 | ggattcgacttcagtagaa | atttatgttggtagtgg | gcgggatccccctattct | cagagcattagcactgc | agggcatcc | caaagctattatgatagta |
| | atgca | tggtggcact | ggttatggctactttaac | a | | gtgctggtaatact |
| | | | ttg | | | |
| | 384 | 385 | 386 | 387 | - | 388 |
| C19 | ggattcaccatcagtagta | atgcatgttggtagtg | gcgagatatgttaatag | cagaccattagcaacct | gatgcatcc | caacagggttggagtagt |
| | ctttctac | ctactaccact | tggttattatatggactc | c | | agtaatgttgataatgtt |
| | | | g | | | |
| | 389 | 390 | 391 | 392 | - | 393 |
| C20 | ggattctccttcagtagca | atccatattagtagtg | gcgagagacgatcgtta | gagagcattagcagttg | caggcatcc | caatctacttattataatg |
| | gccactat | gtgtcaca | tggtcatggtggttatcc | g | | agaatgttggaaatgcg |
| | | | ttatactgacttg | | | |
| | 394 | 395 | 396 | 397 | - | 398 |
| C21 | ggattctccttcagtagca | atttataatggtgatg | gcgagagatgctgatta | gagagcattggcaatgc | gttgcatcc | caatgtacttggtatggta |
| | gctactac | gcagcaca | tgctggcgtggtgctcaa | a | | atgtctatcttgct |
| | | | cttg | | | |
| | 399 | 400 | 401 | 402 | - | 403 |
| C22 | ggattctccttcagtagcg | atttatggtgctgttag | gcgagagatagttatag | gaaaatatttatgtaga | tctgcatcc | gcagggagttttagtagta |
| | gctactac | tgataggatt | tggtagtacttattatag | caaatac | | gtgatagttgtgct |
| | | | atcatactactttaactt | | | |
| | | | g | | | |
| | 404 | 405 | 406 | 407 | - | 408 |
| C23 | ggattcgacttcagtagtt | atttatggtggtaaag | gcgagagactatgttag | gaggacatttatagctta | gatgcatcc | caaggcggttattatagtg |
| | actac | gtaccaca | tagtagtggttattatat | | | gtagtcctactgataattct |
| | | | ggacttg | | | |
| | 409 | 410 | 411 | 412 | - | 413 |
| C24 | ggaatcgacttcagtagc | attgatactgatgattt | gcgaggggagatgctg | cagagcattggtagcta | tatgcctcc | caatgttcttattttggtag |
| | aatgta | tattggcact | gttactatgccttg | t | | tggtgctgtcgatagtcct |
| | 414 | 415 | 416 | 417 | - | 418 |
| C25 | ggattctccttcagtagca | atttatgctggtagtag | gcgagagaccgttatgg | cagaacatttatgtcga | tatgcatcc | gcaggcggttatagtgctg |
| | gctactac | tggtagcaca | tggtggttcttattatata | cagctac | | attcggataatgct |
| | | | ccttactactttgacttg | | | |
| | 419 | 420 | 421 | 422 | - | 423 |
| C27 | ggattctccttcagttaca | attgataatggtgatg | gcgagagggagtagtg | cagagcattggtagctac | gctacatcc | caatgtggttattatgata |
| | ggtactac | gcgacaca | gttataatggctttaact | | | gtaattatgggggtgct |
| | | | tc | | | |
| | 424 | 425 | 426 | 427 | - | 428 |
| C28 | ggattctccttcagtagcg | attggtgctagtagtg | gcgagagtctatcttggt | gagagcgttagtagtag | gatgcatcc | caaaactattatggtatta |
| | gctactac | gactcact | ggtgctgattatgagca | a | | gtggtgtcgct |
| | | | atttaatttg | | | |
| | 429 | 430 | 431 | 432 | - | 433 |
| C29 | ggattctccttcagtgaca | atttatattggttttagt | gcgagaggggttttttat | cagagcattgctagttac | agggcatcc | caaactacttatgttggtg |
| | actactgg | ggtaccact | gttggtagtggttattgg | | | gtagtggcaatgct |
| | | | tactacttgaacttg | | | |
| | 434 | 435 | 436 | 437 | - | 438 |
| C30 | ggattcgacttcagtagct | attgatactggtagtg | gcgagagatccctatttt | cagagtgtttataacaa | aaggcttcc | ctaggcggttatagtagtc |
| | actactac | gtgagact | ggcactaactta | caactac | | ctagtgatgatgct |
| | 439 | 440 | 441 | 442 | - | 443 |
| C53 | ggattctccttcagtagca | atttatcttggtgatga | gcgagacatggatataa | gagagtagtagtcgtca | tgggcatcc | caaagctattatgggctta |
| | gccactat | cagcaca | tcttgctattcataattat | c | | gtaataatggttggagata |
| | | | attata | | | tgct |
| | 444 | 445 | 446 | 447 | - | 448 |
| C55 | ggattctccttcagtagcg | attgatggtggtagta | gcgagatgtatttggagt | gaggacatcgaaaact | agggcatcc | caacagggttatagtggt |
| | actacgac | gtggtgagatt | cataattactatgacttg | at | | agtaatgttgataatttt |
| | 449 | 450 | 451 | 452 | - | 453 |
| C56 | ggattctccttcagtagca | atttatcctagtagtag | gcgagagatattccttat | cagagcattggtagtaa t | tctgcatcc | caatttacttattatggtag |
| | gctactgg | tggtagcact | aatattcatggttatact | | | tagtgatgtcgatgtt |
| | | | accggatttaacttg | | | |
| | 454 | 455 | 456 | 457 | - | 458 |
| C57 | ggattctccttcagtagcg | attggtgctagttatgg | acgagagatgatggtgg | cagagcattggtagtatt | gctgcagcc | caaagctattattatagtg |
| | gctactgg | tggcact | tcttgataatcctaatttg | | | ttaccactagttattcgaat |
| | | | | | | agt |
| | 459 | 460 | 461 | 462 | - | 463 |
| C59 | ggattcaccatcagtagtg | atttatattggtagtgg | gcgagagataagggat | cagagcattggtaaata | tcttcgtcc | caacagggttatagttatg |
| | actactac | tactgatact | ggtctaatgcctggggtt | c | | ttgatcttgaaaatggt |
| | | | tctactttcagttg | | | |
| | 464 | 465 | 466 | 467 | - | 468 |
| C61 | ggattctccttcagtagca | atttatgctggtggtag | gcgagagatggtgttag | cagagcattagtagcta | aaggcatcc | caaagtagttattatggtc |
| | gctactgg | tggtagcact | tggtagtggttggttcttt | c | | ctagtgatgttgatttttct |
| | | | aacttg | | | cct |
| | 469 | 470 | 471 | 472 | - | 473 |
| C64 | ggattctccttcagtaaca | atttatgctggtgatac | gcgagagatggggatg | gagaggattggcaatgc | cagacatcc | caaagctattattatactg |
| | actactgg | tggtaccgct | gtgttgggaatggtggt | a | | gcagtattagttatgaa |
| | | | acttttgccttg | | | |
| | 474 | 475 | 476 | 477 | - | 478 |
| C65 | ggattctccttcagtagca | atttttactggtggtgtt | gcgagaacctatactgc | cagagtattagtagttac | agggcatcc | caaagtgattattatagta |
| | gctactac | agcacg | tggttttactggtgataa | | | gtagtagcaataatggttt |
| | | | ctactttaacttg | | | tgtt |
| | 479 | 480 | 481 | 482 | - | 483 |
| C68 | ggattctccttcagtggcc | attggtgctcgtagta | gcgagatcgtatcctgct | gaagacatttatagttta | gatgcatcc | caaggcggttattatagtg |
| | cctactgg | gtggtagcact | tatgctggtgacggttat | | | gtgatggtactgatatacc t |
| | | | gccctatactactttaac | | | |
| | | | ttg | | | |
| | 484 | 485 | 486 | 487 | - | 488 |

TABLE C
The kinetic binding affinities of CHI3L1 mAbs to human CHI3L1 and mouse CHI3L1

| | hChi3l1 | | | | | mChi3l1 | | | | |
|---|---|---|---|---|---|---|---|---|---|---|
| Antibody | KD (M) | KD Error | kon(1/Ms) | kdis(1/s) | Full R\hat{ }2 | KD (M) | KD Error | kon(1/Ms) | kdis(1/s) | Full R\hat{ }2 |
| C53 | <1.0E−12 | 5.24E−11 | 1.37E+05 | <1.0E−07 | 0.9991 | 3.54E−09 | 8.43E−11 | 7.73E+04 | 2.73E−04 | 0.9994 |
| C55 | n.a. | n.a. | n.a. | n.a. | n.a. | 1.22E−09 | 4.40E−11 | 1.15E+05 | 1.41E−04 | 0.9996 |
| C56 | <1.0E−12 | 5.218E−11 | 1.89E+05 | <1.0E−07 | 0.9985 | 7.92E−09 | 1.83E−10 | 7.48E+04 | 5.92E−04 | 0.9987 |
| C57 | n.a. | n.a. | n.a. | n.a. | n.a. | 4.09E−09 | 1.73E−10 | 5.45E+04 | 2.23E−04 | 0.9988 |
| C59 | 1.418E−09 | 5.843E−11 | 1.66E+05 | 2.35E−04 | 0.9984 | 3.432E−09 | 1.075E−10 | 1.17E+05 | 4.02E−04 | 0.9982 |
| C61 | n.a. | n.a. | n.a. | n.a. | n.a. | 2.81E−09 | 1.08E−10 | 7.15E+04 | 2.01E−04 | 0.9992 |
| C64 | n.a. | n.a. | n.a. | n.a. | n.a. | 2.54E−10 | 3.15E−10 | 1.07E+05 | 2.71E−05 | 0.9862 |
| C65 | n.a. | n.a. | n.a. | n.a. | n.a. | 6.00E−09 | 2.27E−10 | 1.26E+05 | 7.56E−04 | 0.9851 |
| C68 | n.a. | n.a. | n.a. | n.a. | n.a. | 7.99E−10 | 6.70E−11 | 1.38E+05 | 1.10E−04 | 0.9989 |

n.a.: no detectable binding

TABLE D
Epitope bins of the anti-CHI3L1 antibodies

| | Bin | | | | | | | | |
|---|---|---|---|---|---|---|---|---|---|
| Anti- | Bin1 | | | Bin2 | Bin3 | Bin4 | | Bin5 | |
| bodies | C53 | C55 | C56 | C57 | C59 | C61 | C68 | C64 | C65 |
| C53 | + | + | + | | | | | | |
| C55 | + | + | + | | | | | | |
| C56 | + | + | + | | | | | | |
| C57 | | | | + | | | | | |
| C59 | | | | | + | | | | |
| C61 | | | | | | + | + | | |
| C68 | | | | | | + | + | | |
| C64 | | | | | | | | + | + |
| C65 | | | | | | | | + | + |

TABLE E
Grouping of CHI3L1 mAbs based on binding to different
fragments of CHI3L1 protein (AA: 22-256) and
fragment CHI3L1 protein (AA: 257-383)

| Chi3L1 protein | |
|---|---|
| fragments | mAbs binding group |
| AA: 22-256 | C1, C3, C7, C14, C17, C23, C24, C27, C29 |
| AA: 257-383 | C2, C4, C8, C9, C13, C15, C16, C18, C19, |
| | C20, C21, C25, C28, C30 |

### Example 3

Introduction. Hepatocellular carcinoma (HCC) is the fifth most common cancer and the second most common cancer-related death worldwide. It is an aggressive cancer with poor prognosis due to metastases and postsurgical recurrence. Pharmacological treatment choices for HCC are limited. For many years sorafenib, a tyrosine kinase inhibitor, was the only therapy available in advanced HCC. Several additional inultikinase inhibitors have recently been approved by the FDA. An imimunue checkpoint inhibitor, nivolumab (anti-PDI antibody) was also approved by the FDA in 2017 for the treatment of HCC in patients who have progressed on sorafenib. However, only a small portion of patients respond to the treatments.

HCC is an inflammation-driven disease. Up to 80% of HCC patients present with long-term liver inflammation and cirrhosis, suggesting that the tumor microenvironment plays a critical role in HCC progression. The TME of HCC is characterized by an immunosuppressive nature, attributable to the presence of various cells, including tumor-associated macrophages (TAMs), that inhibit anti-tumor T cell responses. Macrophages are categorized into classically activated (M1) and alternatively activated (M2) phenotypes based on cell surface markers and functional characteristics. M2 cells play important roles in promoting wound-healing, angiogenesis, and immunosuppression. These functions are beneficial during tissue repair after injury but facilitate tumor growth and metastasis. Analyses of human HCC samples showed that the majority of TAMs were alternatively activated M2 macrophages. These cells are known to produce a variety of mediators that promote tumor cell growth and angiogenesis. More importantly, TAMs exert important immunosuppressive functions. They produce a set of cytokines and chemokines, including IL-10, TGF-β, CCL17, 18, 22, and 24, to recruit and activate regulatory T cells, which in turn suppress cytotoxic T cell activation.

Chitinase 3-like 1 (also known as YKL-40) is a member of the 18-glycosyl hydrolase family consisting of chitinases and chitinase-like proteins, and it lacks chitinase activities. Although functional studies of Chi3l1 are limited, many clinical reports have described elevated serum levels of Chi3l1 in diseases involving inflammation and tissue remodeling, such as rheumatoid arthritis, inflammatory bowel disease, multiple sclerosis, diabetes, chronic obstructive lung disease, as well as cancers of many tissues including the lung, prostate, colon, breast, brain, kidney and liver.

With regard to HCC, proteomics and transcriptomics analyses revealed higher expression levels of Chi3l1 in the tumors than in peritumoral normal tissues. Moreover, the higher levels of Chi3l1 are associated with advanced tumor-node-metastasis stages, worse overall survival and disease-free survival. In HCC patients receiving curative resection, those with higher levels of Chi3l1 within 6 months after surgery had notably shorter overall survival than those with lower Chi3l1 levels. These findings suggest the potential of Chi3l1 to serve as an independent prognostic marker in HCC patients.

However, the mechanism of the pathophysiological involvement of Chi3l1 in HCC development has not been studied. Some evidence in the literature points toward a link between Chi3l1 and macrophage functions. In human and mouse fibrotic livers, the increase of Chi3l1 serum levels coincides with the increase of liver macrophages. It is shown that Chi3l1 inhibits apoptosis of macrophages in the liver and lung. Moreover, Chi3l1 is reported to promote alternative activation of peritoneal, alveolar, dermal and intestinal macrophages.

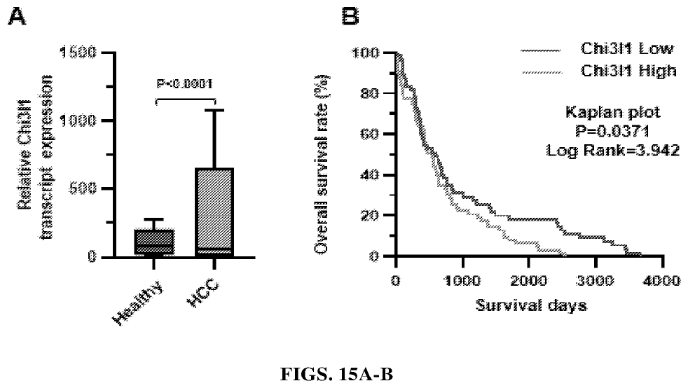

Increased Chi3l1 expression correlates with HCC severity. Analyses of Cancer Genome Atlas (TCGA) database revealed higher transcript levels of Chi31l in HCC patients than healthy controls (FIG. 15). Moreover, HCC patients with a high expression of Chi31l correlates with a lower survival rate compared to those with a low expression of Chi31l (FIG. 15).

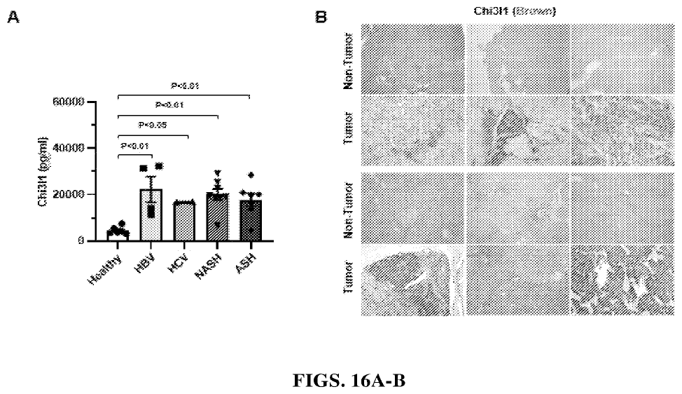

The inventors measured Chi31l protein levels in the plasma of HCC patients with different etiologies including HBV, HCV, NASH, and ASH. These data showed that regardless of the etiologies, the plasma levels of Chi31l protein were higher compared to healthy controls (FIGS. 16A-B). They also found that Chi31l was highly expressed in the tumors but not in adjacent normal liver tissues from HCC patients (FIGS. 16A-B).

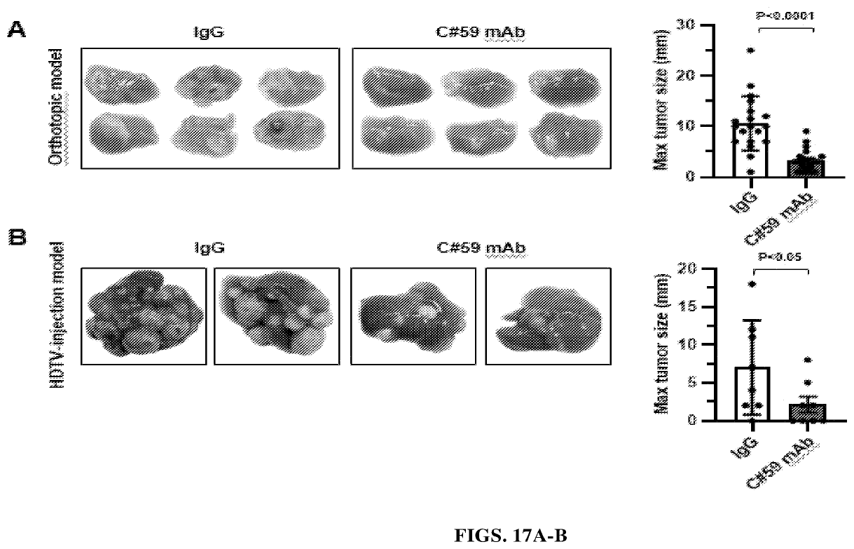

Treatment with anti-Chi3l1 monoclonal antibody (mAb) inhibits HCC progression. The inventors generated a panel of anti-Chi31l monoclonal antibodies (mAb). They found clone 59 (C59mAb) could markedly reduce HCC development using two mouse models of HCC (FIGS. 17A-B). In the first model, they implanted Chi31l-expressing Hepa 1-6 cells directly into the mouse liver (orthotopic model). After 1-week, mice were randomly divided into two groups treated with either IgG or α-Chi31l (200 μg/mouse) twice per week for 4 weeks. In the second model, the inventors introduced oncogenes (0-catenin and c-met) by hydrodynamic injection into the tail vein. Four weeks after the injection, mice were randomly divided into two groups treated with either IgG or α-Chi31l (200 μg/mouse) twice per week for additional 4 weeks. Data shown in FIGS. 17A-B suggest that neutralizing Chi31l by C59mAb effectively reduced tumor growth.

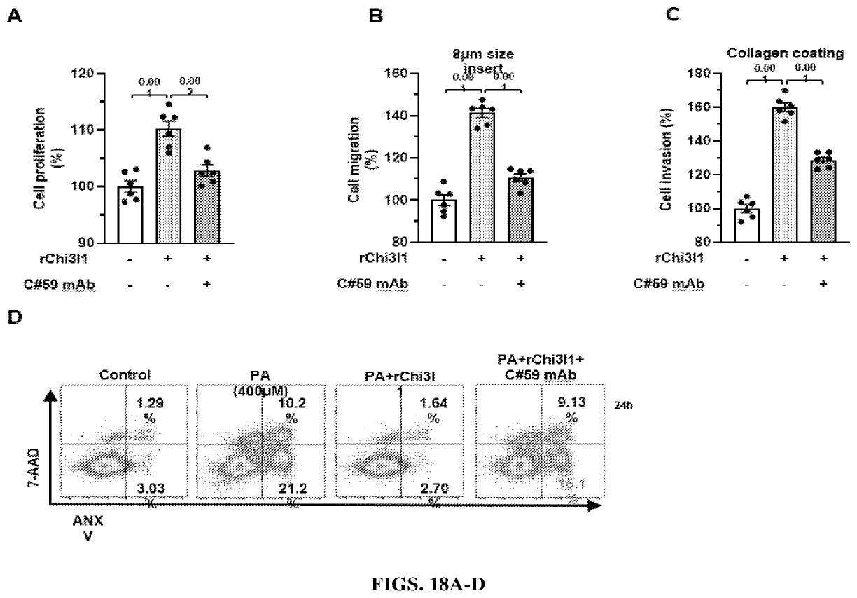

Determine the direct effects of Chi3l1 on tumor cells in vitro. The inventors treated hepal-6 cells with recombinant Chi31l protein (rChi31l) in the absence or presence of C59mAb in in vitro cell cultures. The data showed that cell proliferation, migration and invasion were significantly increased by rChi3l1 and that the effects of rChi3l1 were reduced by C59mAb (FIGS. 18A-B). Cells were treated with palmitic acid (PA) to induce apoptosis and analyzed by annexin V (ANX V) staining. The data showed that rChi3l1 inhibited apoptosis, which is reversed by C59mAb (FIGS. 18A-B).

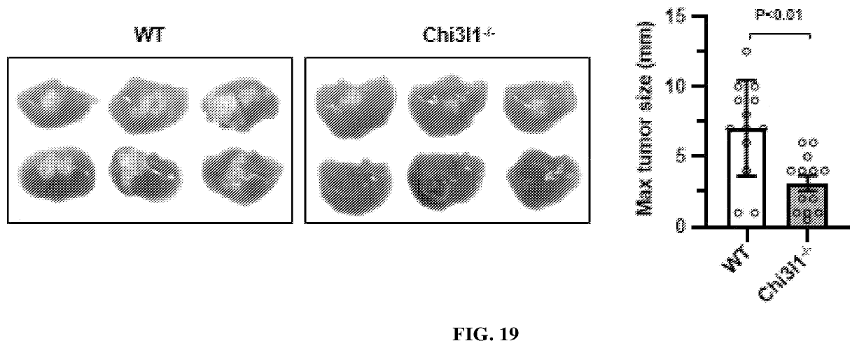

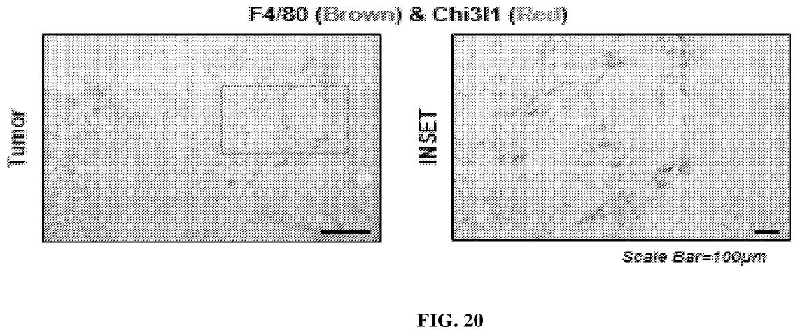

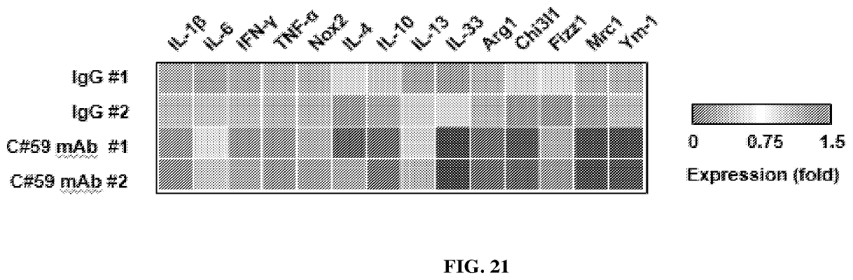

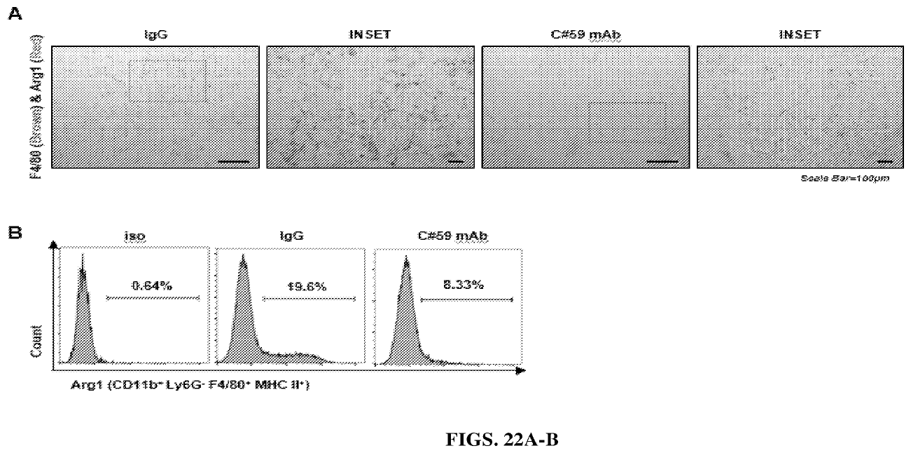

C59mAb suppresses the pro-tumorigenic phenotype of tumor-associated macrophages (TAMs). The inventors implanted hepa1-6 cells to the livers of WT and Chi3l1^{-/-} mice. The data showed that Chi3l1^{-/-} mice, which are deficient of Chi3l1 in tumor microenvironment but not in implanted tumor cells, developed significantly smaller tumors than WT mice (FIG. 19). This finding suggests that the effect of Chi3l1 on tumor microenvironment plays a critical role in its pro-tumorigenic function. Moreover, immunohistochemical staining (IHC) demonstrated that TAMs expressed Chi3l1 (FIG. 20). Comparison of gene expression profiles of TAMs from IgG- and C59mAb-treated mice unveiled that C59mAb suppressed M2-related genes, including Ym-1, Fizz1, Mrcl and arginase (Arg)1 (FIG. 21). IHC staining of Arg 1 and F4/80 (marker of macrophages) showed that ArgI was expressed by TAMs and that the expression levels were reduced in TAMs from C59mAb-treated mice (FIGS. 22A-B).

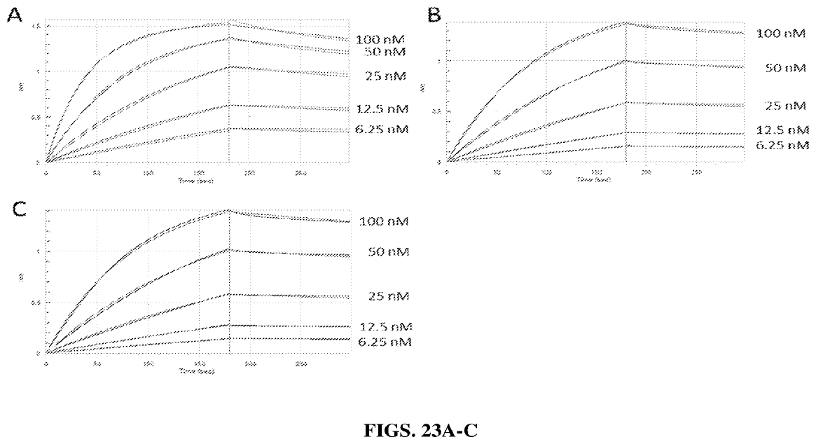

C59mAb enhances anti-tumor T cell responses. In the orthotopic mouse model of HCC, the inventors isolated immune cells from IgG- and C59mAb-treated mice. They investigated CD4+ and CD8+ activations by using flow cytometry to measure their production of TNFa and IFNg. As shown in FIGS. 23A-C, C59mAb treatment resulted in enhanced TNFa and IFNg expression by both CD4+ and CD8+ T cells.

Summary. These findings suggest that Chi3l1 promotes HCC progression through inducing a pro-tumorigenic phenotype of TAMs and thus suppressing anti-tumor T cell responses. Importantly, the inventors demonstrated that neutralizing Chi3l1 using C59mAb could inhibit the pro-tumorigenic effects of TAMs, thereby enhancing anti-tumor T cell immunity. These studies provide mechanistic insights into the pro-tumorigenic function of Chi3l1 and identify Chi3l1 as not only a biomarker but also a viable therapeutic target in treating HCC.

TABLE F
Kinetic binding parameters of C59Hu and
C7Hu determined using Octet BLI method

| mAb | Antigen | KD (M) | kon(1/Ms) | kdis(1/s) | R\hat{ }2 |
|---|---|---|---|---|---|
| C59Hu | Mouse | 4.44E−09 | 2.14E+05 | 9.50E−04 | 0.97 |
| | Chi3L1 | | | | |
| C59Hu | Human | 4.50E−09 | 1.41E+05 | 6.35E−04 | 0.98 |
| | Chi3L1 | | | | |
| C7Hu | Human | 8.80E−10 | 5.59E+05 | 4.92E−04 | 0.99 |
| | Chi3L1 | | | | |

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. Bernal, W., Auzinger, G., Dhawan, A. & Wendon, J. Acute liver failure. Lancet 376, 190-201, doi:10.1016/50140-6736(10)60274-7 (2010).
2. Stravitz, R. T. et al. Thrombocytopenia Is Associated With Multi-organ System Failure in Patients With Acute Liver Failure. Clin Gastroenterol Hepatol 14, 613-620 e614, doi:10.1016/j.cgh.2015.09.029 (2016).
3. European Association for the Study of the Liver. Electronic address, e. e. e. et al. EASL Clinical Practical Guidelines on the management of acute (fulminant) liver failure. J Hepatol 66, 1047-1081, doi:10.1016/j.jhep.2016.12.003 (2017).
4. Stravitz, R. T. et al. Role of procoagulant microparticles in mediating complications and outcome of acute liver injury/acute liver failure. Hepatology 58, 304-313, doi:10.1002/hep.26307 (2013).
5. Harrison, P. M., O'Grady, J. G., Keays, R. T., Alexander, G. J. & Williams, R. Serial prothrombin time as prognostic indicator in paracetamol induced fulminant hepatic failure. BMJ 301, 964-966 (1990).
6. Kerr, R. et al. Effects of acute liver injury on blood coagulation. J Thromb Haemost 1, 754-759 (2003).
7. Miyakawa, K. et al. Platelets and protease-activated receptor-4 contribute to acetaminophen-induced liver injury in mice. Blood 126, 1835-1843, doi:10.1182/blood-2014-09-598656 (2015).
8. Larson, A. M. et al. Acetaminophen-induced acute liver failure: results of a United States multicenter, prospective study. Hepatology 42, 1364-1372, doi:10.1002/hep.20948 (2005).
9. Lalor, P. F., Herbert, J., Bicknell, R. & Adams, D. H. Hepatic sinusoidal endothelium avidly binds platelets in an integrin-dependent manner, leading to platelet and endothelial activation and leukocyte recruitment. Am J Physiol Gastrointest Liver Physiol 304, G469-478, doi:10.1152/ajpgi.00407.2012 (2013).
10. Lang, P. A. et al. Aggravation of viral hepatitis by platelet-derived serotonin. Nat Med 14, 756-761, doi:10.1038/nm1780 (2008).
11. Lesurtel, M. et al. Platelet-derived serotonin mediates liver regeneration. Science 312, 104-107, doi:10.1126/science.1123842 (2006).
12. Wong, C. H., Jenne, C. N., Petri, B., Chrobok, N. L. & Kubes, P. Nucleation of platelets with blood-borne pathogens on Kupffer cells precedes other innate immunity and contributes to bacterial clearance. Nat Immunol 14, 785-792, doi:10.1038/ni.2631 (2013).
13. Malehmir, M. et al. Platelet GPIba is a mediator and potential interventional target for NASH and subsequent liver cancer. Nature Medicine 25, 641-655, doi:10.1038/s41591-019-0379-5 (2019).
14. Lee, C. G. et al. Role of chitin and chitinase/chitinase-like proteins in inflammation, tissue remodeling, and injury. Annu Rev Physiol 73, 479-501, doi:10.1146/annurev-physiol-012110-142250 (2011).
15. Hakala, B. E., White, C. & Recklies, A. D. Human cartilage gp-39, a major secretory product of articular chondrocytes and synovial cells, is a mammalian member of a chitinase protein family. J Biol Chem 268, 25803-25810 (1993).
16. Kirkpatrick, R. B. et al. Induction and expression of human cartilage glycoprotein 39 in rheumatoid inflammatory and peripheral blood monocyte-derived macrophages. Exp Cell Res 237, 46-54, doi:10.1006/excr.1997.3764 (1997).
17. Kawada, M., Hachiya, Y., Arihiro, A. & Mizoguchi, E. Role of mammalian chitinases in inflammatory conditions. Keio J Med 56, 21-27 (2007).
18. Lee, C. G. et al. Role of breast regression protein 39 (BRP-39)/chitinase 3-like-1 in Th2 and IL-13-induced tissue responses and apoptosis. J Exp Med 206, 1149-1166, doi:10.1084/jem.20081271 (2009).
19. Ponta, H., Sherman, L. & Herrlich, P. A. CD44: from adhesion molecules to signalling regulators. Nat Rev Mol Cell Biol 4, 33-45, doi:10.1038/nrm1004 (2003).
20. Ahrens, T. et al. Soluble CD44 inhibits melanoma tumor growth by blocking cell surface CD44 binding to hyaluronic acid. Oncogene 20, 3399-3408, doi:10.1038/sj.onc.1204435 (2001).
21. Alaniz, L. et al. Interaction of CD44 with different forms of hyaluronic acid. Its role in adhesion and migration of tumor cells. Cell Commun Adhes 9, 117-130 (2002).
22. Bartolazzi, A., Peach, R., Aruffo, A. & Stamenkovic, I. Interaction between CD44 and hyaluronate is directly implicated in the regulation of tumor development. J Exp Med 180, 53-66 (1994).
23. Blass, S. L., Pure, E. & Hunter, C. A. A role for CD44 in the production of IFN-gamma and immunopathology during infection with Toxoplasma gondii. J Immunol 166, 5726-5732 (2001).
24. Kittl, E. M. et al. Serum levels of soluble CD44 variant isoforms are elevated in rheumatoid arthritis. Rheumatol Int 16, 181-186 (1997).
25. Naor, D. & Nedvetzki, S. CD44 in rheumatoid arthritis. Arthritis Res Ther 5, 105-115 (2003).
26. Sato, M. I., Koshino, T. & Takagi, T. CD44 expression on chondrocytes in knees of DBA/1 mice with type II collagen-induced arthritis. Clin Exp Rheumatol 17, 185-190 (1999).
27. Lucarini, G. et al. Involvement of vascular endothelial growth factor, CD44 and CD133 in periodontal disease and diabetes: an immunohistochemical study. J Clin Periodontol 36, 3-10, doi:10.1111/j.1600-051X.2008.01338.x (2009).
28. Slevin, M. et al. Hyaluronan-mediated angiogenesis in vascular disease: uncovering RHAMM and CD44 receptor signaling pathways. Matrix Biol 26, 58-68, doi:10.1016/j.matbio.2006.08.261 (2007).
29. Wang, J. & Kubes, P. A Reservoir of Mature Cavity Macrophages that Can Rapidly Invade Visceral Organs to Affect Tissue Repair. Cell 165, 668-678, doi:10.1016/j.cell.2016.03.009 (2016).
30. Rahman, T. M. & Hodgson, H. J. Animal models of acute hepatic failure. Int J Exp Pathol 81, 145-157, doi:10.1046/j.1365-2613.2000.00144.x (2000).
31. Mosser, D. M. & Edwards, J. P. Exploring the full spectrum of macrophage activation. Nat Rev Immunol 8, 958-969, doi:10.1038/nri2448 (2008).
32. Mitchell, J. R. et al. Acetaminophen-induced hepatic necrosis. I. Role of drug metabolism. J Pharmacol Exp Ther 187, 185-194 (1973).
33. Jollow, D. J. et al. Acetaminophen-induced hepatic necrosis. II. Role of covalent binding in vivo. J Pharmacol Exp Ther 187, 195-202 (1973).
34. Cover, C. et al. Peroxynitrite-induced mitochondrial and endonuclease-mediated nuclear DNA damage in acetaminophen hepatotoxicity. J Pharmacol Exp Ther 315, 879-887, doi:10.1124/jpet.105.088898 (2005).
35. Chiu, H. et al. Role of p55 tumor necrosis factor receptor 1 in acetaminophen-induced antioxidant defense. Am J Physiol Gastrointest Liver Physiol 285, G959-966, doi:10.1152/ajpgi.00219.2003 (2003).
36. Gardner, C. R. et al. Reduced hepatotoxicity of acetaminophen in mice lacking inducible nitric oxide synthase: potential role of tumor necrosis factor-alpha and interleukin-10. Toxicol Appl Pharmacol 184, 27-36 (2002).
37. Hinson, J. A., Reid, A. B., McCullough, S. S. & James, L. P. Acetaminophen-induced hepatotoxicity: role of metabolic activation, reactive oxygen/nitrogen species, and mitochondrial permeability transition. Drug Metab Rev 36, 805-822, doi:10.1081/DMR-200033494 (2004).
38. Jaeschke, H., Knight, T. R. & Bajt, M. L. The role of oxidant stress and reactive nitrogen species in acetaminophen hepatotoxicity. Toxicol Lett 144, 279-288 (2003).
39. Hamburger, S. A. & McEver, R. P. GMP-140 mediates adhesion of stimulated platelets to neutrophils. Blood 75, 550-554 (1990).
40. Larsen, E. et al. PADGEM protein: a receptor that mediates the interaction of activated platelets with neutrophils and monocytes. Cell 59, 305-312 (1989).
41. Simon, D. I. et al. Platelet glycoprotein ibalpha is a counterreceptor for the leukocyte integrin Mac-1 (CD11b/CD18). J Exp Med 192, 193-204 (2000).
42. Weber, C. & Springer, T. A. Neutrophil accumulation on activated, surface-adherent platelets in flow is mediated by interaction of Mac-1 with fibrinogen bound to alphaIIbbeta3 and stimulated by platelet-activating factor. J Clin Invest 100, 2085-2093, doi:10.1172/JCI119742 (1997).
43. Kerrigan, A. M. et al. Podoplanin-expressing inflammatory macrophages activate murine platelets via CLEC-2. J Thromb Haemost 10, 484-486, doi:10.1111/j.1538-7836.2011.04614.x (2012).
44. Diacovo, T. G., deFougerolles, A. R., Bainton, D. F. & Springer, T. A. A functional integrin ligand on the surface of platelets: intercellular adhesion molecule-2. J Clin Invest 94, 1243-1251, doi:10.1172/JCI117442 (1994).
45. Iannacone, M. et al. Platelets mediate cytotoxic T lymphocyte-induced liver damage. Nat Med 11, 1167-1169, doi:10.1038/nm1317 (2005).
46. Geng, B. et al. Chitinase 3-like 1-CD44 interaction promotes metastasis and epithelial-to-mesenchymal transition through beta-catenin/Erk/Akt signaling in gastric cancer. J Exp Clin Cancer Res 37, 208, doi:10.1186/s13046-018-0876-2 (2018).
47. Zhou, Y. et al. Chitinase 3-like-1 and its receptors in Hermansky-Pudlak syndrome-associated lung disease. J Clin Invest 125, 3178-3192, doi:10.1172/JCI79792 (2015).
48. He, C. H. et al. Chitinase 3-like 1 regulates cellular and tissue responses via IL-13 receptor alpha2. Cell Rep 4, 830-841, doi:10.1016/j.celrep.2013.07.032 (2013).
49. Lee, C. M. et al. IL-13Ralpha2 uses TMEM219 in chitinase 3-like-1-induced signalling and effector responses. Nat Commun 7, 12752, doi:10.1038/ncomms12752 (2016).
50. Zhou, Y. et al. Galectin-3 Interacts with the CHI3L1 Axis and Contributes to Hermansky-Pudlak Syndrome Lung Disease. J Immunol 200, 2140-2153, doi:10.4049/jimmunol.1701442 (2018).
51. Hitchcock, J. R. et al. Inflammation drives thrombosis after Salmonella infection via CLEC-2 on platelets. J Clin Invest 125, 4429-4446, doi:10.1172/JCI79070 (2015).
52. Laskin, D. L., Gardner, C. R., Price, V. F. & Jollow, D. J. Modulation of macrophage functioning abrogates the acute hepatotoxicity of acetaminophen. Hepatology 21, 1045-1050 (1995).
53. Michael, S. L., Pumford, N. R., Mayeux, P. R., Niesman, M. R. & Hinson, J. A.

Pretreatment of mice with macrophage inactivators decreases acetaminophen hepatotoxicity and the formation of reactive oxygen and nitrogen species. Hepatology 30, 186-195, doi:10.1002/hep.510300104 (1999).
54. Ju, C. et al. Protective role of Kupffer cells in acetaminophen-induced hepatic injury in mice. Chem Res Toxicol 15, 1504-1513 (2002).
55. Guerrero Munoz, F. & Fearon, Z. Sex related differences in acetaminophen toxicity in the mouse. J Toxicol Clin Toxicol 22, 149-156 (1984).
56. Holt, M. P., Cheng, L. & Ju, C. Identification and characterization of infiltrating macrophages in acetaminophen-induced liver injury. J Leukoc Biol 84, 1410-1421, doi:10.1189/jlb.0308173 (2008).
57. Shan, Z. et al. Chitinase 3-like-1 promotes intrahepatic activation of coagulation through induction of tissue factor in mice. Hepatology 67, 2384-2396, doi:10.1002/hep.29733 (2018).
58. Deng, M. et al. LILRB4 signalling in leukaemia cells mediates T cell suppression and tumour infiltration. Nature 562, 605-609, doi:10.1038/s41586-018-0615-z (2018).

1. An isolated monoclonal antibody or an antigen-binding fragment thereof comprising cloned paired heavy and light chain CDRs from Table A.

2. The isolated monoclonal antibody or an antigen-binding fragment thereof of claim 1, wherein antibody or fragment thereof is encoded by clone-paired heavy and light chain sequences from FIGS. 24, 25 and 29.

3. The isolated monoclonal antibody or an antigen-binding fragment thereof of claim 1, wherein antibody or fragment thereof is encoded by heavy and light chain variable sequences having at least 70%, 80%, 90% or 95% identity to clone-paired sequences from FIGS. 24, 25 and 29.

4. The isolated monoclonal antibody or an antigen-binding fragment thereof of claim 1, wherein antibody or fragment thereof comprises clone-paired heavy and light chain sequences from FIGS. 26, 27 and 28.

5. The isolated monoclonal antibody or an antigen-binding fragment thereof of claim 1, wherein antibody or fragment thereof comprises heavy and light chain variable sequences having at least 70%, 80%, 90% or 95% identity to clone paired sequences from FIGS. 26, 27 and 28.

6. The isolated monoclonal antibody or an antigen binding fragment thereof of claim 1, wherein the isolated monoclonal antibody is a murine, a rodent, or a rabbit.

7. The isolated monoclonal antibody or an antigen binding fragment thereof of claim 1, wherein the isolated monoclonal antibody is a humanized, or human antibody.

8. The isolated monoclonal antibody or an antigen-binding fragment thereof of claim 1, wherein the antigen-binding fragment is a recombinant ScFv (single chain fragment variable) antibody, Fab fragment, F(ab′)2 fragment, or Fv fragment.

9. The isolated monoclonal antibody or an antigen binding fragment thereof of claim 1, wherein the isolated monoclonal antibody is a bispecific antibody or a chimeric antibody.

10. The isolated monoclonal antibody or antigen binding fragment thereof of claim 1, wherein said antibody is an IgG, or a recombinant IgG antibody or antibody fragment comprising an Fc portion mutated to alter (eliminate or enhance) FcR interactions, to increase half-life and/or increase therapeutic efficacy, such as a LALA, N297, GASD/ALIE, YTE or LS mutation or glycan modified to alter (eliminate or enhance) FcR interactions such as enzymatic or chemical addition or removal of glycans or expression in a cell line engineered with a defined glycosylating pattern.

11. An isolated monoclonal antibody or an antigen binding fragment thereof, which competes for the same epitope with the isolated monoclonal antibody or an antigen-binding fragment thereof according to claim 1.

12. A pharmaceutical composition comprising the isolated monoclonal antibody or an antigen-binding fragment thereof according to claim 1, and a pharmaceutically acceptable carrier.

13. An isolated nucleic acid that encodes the isolated monoclonal antibody according to claim 1.

14-24. (canceled)

25. A method of treating or ameliorating the effect of a cancer in a subject, the method comprising administering to the subject a therapeutically effective amount of the antibody or an antigen-binding fragment thereof according to claim 1.

26. The method of claim 25, wherein the method reduces or eradicates the tumor burden in the subject, reduces the number of tumor cells, reduces tumor size, or eradicates the tumor in the subject.

27-29. (canceled)

30. The method of claim 25, wherein the cancer is a solid tumor cancer such as lung cancer, brain cancer, skin cancer, head and neck cancer, liver cancer, pancreatic cancer, stomach cancer, bladder cancer, colon cancer, testicular cancer, cervical cancer, breast cancer, or uterine cancer.

31. The method of claim 30, wherein the cancer is a hematologic malignancy, such as myelodysplastic syndromes, myeloproliferative neoplasms, chronic myelomonocytic leukemia (CMML), chronic myelocytic leukemia, or acute myeloid leukemia (AML), acute promyelocytic leukemia (APL) or M3 AML, acute myelomonocytic leukemia or M4 AML, acute monocytic leukemia or M5 AML, acute myeloblastic leukemia, or polycythemia vera.

32. The method of claim 25, wherein the antibody or an antigen-binding fragment thereof is administered intravenously, intra-arterially, intra-tumorally, or subcutaneously.

33. The method of claim 25, further comprising administering to the subject a second anti-cancer therapy, such as one or more drugs selected from the group consisting of a topoisomerase inhibitor, an anthracycline topoisomerase inhibitor, an anthracycline, a daunorubicin, a nucleoside metabolic inhibitor, a cytarabine, a hypomethylating agent, a low dose cytarabine (LDAC), a combination of daunorubicin and cytarabine, a daunorubicin and cytarabine liposome for injection, Vyxeos®, an azacytidine, Vidaza®, a decitabine, an all-trans-retinoic acid (ATRA), an arsenic, an arsenic trioxide, a histamine dihydrochloride, Ceplene®, an interleukin-2, an aldesleukin, Proleukin®, a gemtuzumab ozogamicin, Mylotarg®, an FLT-3 inhibitor, a midostaurin, Rydapt®, a clofarabine, a farnesyl transferase inhibitor, a decitabine, an IDH1 inhibitor, an ivosidenib, Tibsovo®, an IDH2 inhibitor, an enasidenib, Idhifa®, a smoothened (SMO) inhibitor, a glasdegib, an arginase inhibitor, an IDO inhibitor, an epacadostat, a BCL-2 inihbitor, a venetoclax, Venclexta®, a platinum complex derivative, oxaliplatin, a kinase inhibitor, a tyrosine kinase inhibitor, a PI3 kinase inhibitor, a BTK inhibitor, an ibrutinib, IMBRUVICA®, an acalabrutinib, CALQUENCE®, a zanubrutinib, a PD-1 antibody, a PD-L1 antibody, a CTLA-4 antibody, a LAG3 antibody, an ICOS antibody, a TIGIT antibody, a TIM3 antibody, a CD40 antibody, a 4-1BB antibody, a CD47 antibody, a SIRPil antibody or fusions protein, an antagonist of E-selectin, an antibody binding to a tumor antigen, an antibody binding to a T-cell surface marker, an antibody binding to a myeloid cell or NK cell surface marker, an alkylating agent, a nitrosourea agent, an antimetabolite, an antitumor antibiotic, an alkaloid derived from a plant, a hormone therapy medicine, a hormone antagonist, an aromatase inhibitor, and a P-glycoprotein inhibitor.

34-44. (canceled)

45. The method according to claim 25, wherein said isolated monoclonal antibody or an antigen binding fragment thereof is conjugated to a liposome or nanoparticle.

46. A method of treating a subject having or at risk of hepatotoxicity comprising administering an antibody according to claim 1 to said subject.

47-55. (canceled)


SEQUENCE LISTING (488 sequences)

SEQ ID NO: 1 - DNA, 345 bp, Artificial sequence
  Synthetic antibody
caggagcagctggtggaggagtccgggggaggcctggtccagcctgggggatccctgacactctcctgcacagcctctgggtgggacttaagtgatacctactggatatgctgggtccgccaggctccagggaagggattggagtggatcggatgcatttataatggtaatggtgacacttactacgcgacctgggcgaaaggccgattcaccatctccaaagcctcgaccacggtggatctgaaaatgaccagtctgacagccgcggacacggccacctatttctgtgcgagaggagtggctgcggacttgtggggcccaggcaccctggtcaccgtctcctca

SEQ ID NO: 2 - DNA, 369 bp, Artificial sequence
  Synthetic antibody
caggagcagctgaaggaggagtccgggggaggcctggtccagcctgagggatccctgacactcacctgcacagcttctggattctccttcagtagcggctattacatgtgctgggtccgccaggctccagggaaggggctggagtggagcgcgtgcattggtgctagtagtggactcacttatttcgcgagctgggcgaaaggccgattcaccatctccaaaacctcgtcgaccacggtgactctgcgaatgaccagtctcacggccgcggacacggccacctatttctgtgcgagagtctatcttggtggtgctgattatgagcgatttaatttgtggggtccaggcaccctggtcaccgtctcttca

SEQ ID NO: 3 - DNA, 360 bp, Artificial sequence
  Synthetic antibody
cagaagcagctggtggagaccgggggaggcctggtccagcctgggggatccctgacactctcctgcaaagtctctgaattcgacttcagtagctactacatacactgggtccgccaggctccagggaaggggctggagtggatcggaatcatttatgctggtaaaggtgacacagactacgcgagctgggtgaatggccgattcaccatctccagcgacaacgcccagaacactgtggatcttcaaatgaacagtctgacagcggcggacacggccacctatttctgtgcgagagggtggctggggtccgattactactttaacttttggggcccaggcaccctggtcaccgtctcctca

SEQ ID NO: 4 - DNA, 351 bp, Artificial sequence
  Synthetic antibody
caggagcagctggtggagtccgggggaggcctggtccagcctgagggatccctgacactcacctgcaaagcctctgggttagacttcagtagcagctactggatatgctgggtccgccaggctccagggaaggggctggagtggatcggatacattaatactagtagtggtagcacatggtacgcgagctgggcgaaaggccgattcaccatctccaaaacctcgtcgaccacggtgactctgcaaatgaccagtctgacagccgcggacacggccatttatttctgtgcgaaatatattaatagttatttcggcttgtggggcccaggcaccctggtcaccgtctcttca

SEQ ID NO: 5 - DNA, 348 bp, Artificial sequence
  Synthetic antibody
cagcagctggtggagtccgggggagacctggtcaagcctgagggatccctgacactcacctgcacagcctctggattcaccctcactacctactgggtgtgctgggtccgccaggctccagggaaggggctggagtggatcgcatgcattgatgctggtagtatcacttactacacgacctgggcgagaggccgattcaccgtctccagaacctcgtcgaccacggtgactctgcaaatgaccagtctgacagccgcggacacggccacctatttctgtgcgagagatgctcattatacttatgcccttgtcttgtggggcccaggcaccctggtcaccgtctcctca

SEQ ID NO: 6 - DNA, 363 bp, Artificial sequence
  Synthetic antibody
cagcagctgaaggagtccgggggagacctggtcaagcctggggcatccctgacactcacctgcacagcctctggattctccttcagtaataataactacatgtgctgggtccgccaggctccagggaaggggctggagtgggtcgcatgcatttatactggtgatgcttacacttacttcgcgagctgggcgaaaggccgattcaccatctccaaaacctcgtcgaccacggtgactctgcaaatgaccagtctgacagccgcggacacggccacctatttctgtgcgagaggttacagtggttatagtggttatggttattccgacttgtggggcccaggcaccctggtcaccgtctcttca

SEQ ID NO: 7 - DNA, 357 bp, Artificial sequence
  Synthetic antibody
cagtcggtggaggagtccgggggagacctggtcaagcctggggcatccctgacactcacctgcacagcctctggattctccttcagtagcagctactggatgtgctgggtccgccaggctccagggaaggggctggagtggatcgcatgcatttatactggtgatggtgctaattactacgcgagctgggcgaaaggccgattcaccatctccaaagcctcgtcgaccacggtgactctacaaatgaccagtctgacagccgcggacacggccacctatttctgtgcgagatctgcttatggtggttatggttattttaacttctggggcccaggcaccctggtcactgtctcttca

SEQ ID NO: 8 - DNA, 363 bp, Artificial sequence
  Synthetic antibody
cagtcagtgaaggagtccgggggagacctggtcaagcctggggcatccctgacactcacctgcacagcctctggattctccttcagtggtgcctactggatatgctgggtccgccaggctccagggaaggggctggaggtgatcgcatgcatttatgtgattagtagtagtaacacttattacgcgagctgggcgaaaggccgattcacctgctccaaaacctcgtcgaccacggtggatctgaagatgaccagtctgacagccgcggacacggccacctatttctgtgcgagatattcttgtagtagtgctttttgcctttttaacttgtggggccctggcaccctggtcaccgtctcttca

SEQ ID NO: 9 - DNA, 360 bp, Artificial sequence
  Synthetic antibody
cagaagcagctggtggagtccgggggaggcctggtccagcctgagggatccctgacactcacctgcaaagcctctggattcgacttcagtagcaatgcaatgtgctgggtccgccaggctccagggaaggggctggagttgctcgcatgctttgattatattagtgatagtatatggtacgcgagctgggtgaatggccgattcaccatctccaaaacctcgtcgaccacggtgactctgcaaatgaccagtctgacagccgcggacacggccacctatttctgtgcgagagacaatgttggtgatggttatactacatttgacttgtggggcccaggcaccctggtcactgtctcttca

SEQ ID NO: 10 - DNA, 351 bp, Artificial sequence
  Synthetic antibody
cagtcagtgaaggagtccgggggaggcctggtccagcctgagggatccctgacactcacctgcacagcctctggattcgacttcagtagcaatgcaatgtgctgggtccgccaggctccagggaaggggcctgagtggatcgcatgcatttataatggtgatgccagtacatactacgcgagctgggcgaaaggccgatctaccatctccaaaacctcgtcgaccacggtgactctgcaaatgaccagtctgacagccgcggacacggccacctatttctgtgcgagatgttttaatagattttacagaggagacttgtggggcccaggcaccctggtcaccgtctcttca

SEQ ID NO: 11 - DNA, 347 bp, Artificial sequence
  Synthetic antibody
cgaagcagctggtggagtccgggggagacctggtcaagcctgagggatccctgacactcacctgcacagcctctggactcaccctcagtagatactggatatgctgggtccgccaggctccagggaaggggctggagtggatcgcatgcattgatgctggtagtatcacttactacgcgagctgggcgaaaggccgattcaccatctccaaaacctcgagcacggtgactctgcaaatgaccagtctgacagccgcggacacggccacctatttctgtgcgagagatgctgattatacttatgcacttgtcttgtggggcccaggcaccctggtcaccgtctcttca

SEQ ID NO: 12 - DNA, 369 bp, Artificial sequence
  Synthetic antibody
cagtcagtgaaggagtccgggggagacctggtcaagcctggggcatccctgacactcacctgcacagcctctggattctccttcagtagcagctactggatatgctgggtccgccaggctccagggaaggggctggagtggatcgcatgcattgatgctggtagtagtgagagcacttactacgcgagctgggcgaaaggccgattcaccatctccaaaacctcgtcgaccacggtgactctgcaaatgaccagtctgacagccgcggacacggccacctatttctgtgcgagagattctagggtagtggctggggtggggatgtacttgaatttgtggggcccaggcaccctcgtcaccgtctcttca

SEQ ID NO: 13 - DNA, 360 bp, Artificial sequence
  Synthetic antibody
aagcagctggtggaggagtccgggggagacctggtcaggcctgagggatccctgacactcacctgcaaaggctctggattcgacttcagtagaaatgcaatgtgctgggtccgccaggctccagggaaggggctggagtggatcgcatgcatttatgttggtagtggtggtggcacttactacgcgacctgggcgaaaggccgattcaccatctccaaaacctcgtcgaccacggtgactctgcaaatgaccagtctgacagccgcggacacggccacctatttctgtgcgggatccccctattctggttatggctactttaacttgtggggcccaggcaccctggtcactgtctcttca

SEQ ID NO: 14 - DNA, 360 bp, Artificial sequence
  Synthetic antibody
caggagcagctggtggagtacgggggagacctggtccagcctgagggatccctgacactcacctgcacagcctctggattcaccatcagtagtactttctacatgggctgggtccgacaggctccagggaaggggctggagtggatcggagacatgcatgttggtagtgctactaccacttactacgcgagctgggcgaaaggccgattcaccatctccaagccctcgtcgaccacggtgactctgcaaatgaccagtctgacagccgcggacacggccacctatttctgtgcgagatatgttaatagtggttattatatggactcgtggggcccaggcaccctggtcactgtctcttca

SEQ ID NO: 15 - DNA, 366 bp, Artificial sequence
  Synthetic antibody
cagtcggtggaggagtccgggggagacctggtcaagcctggggcatccctgacactcacctgcacagcctctggattctccttcagtagcagccactatatgtggtgggtccgccaggctccagggaaggggttggagtggatcggatgcatccatattagtagtggtgtcacatggtacgcgagctgggcgaaaggccgattcaccatctccaaaacctcgtcgaccacggtgactctccaaatgaccagtctgacagccgcggacacggccacctatttctgtgcgagagacgatcgttatggtcatggtggttatccttatactgacttgtggggcccaggcaccctggtcaccgtctcttca

SEQ ID NO: 16 - DNA, 357 bp, Artificial sequence
  Synthetic antibody
cagtcagtgaaggagtccgggggaggcctggtccagcctgagggatccctgacactcacctgcacagcctctggattctccttcagtagcagctactacatgtgctgggtccgccaggctccagggaaggggctggagtggatcgcatgcatttataatggtgatggcagcacatactacgcgagctgggcgaaaggccgattcaccatctccaaaacctcgtcgaccacggtgactctgcaaatgaccagtctgacagccgcggacacggccacctatttctgtgcgagagatgctgattatgctggcgtggtgctcaacttgtggggcccaggtaccctggtcactgtctcttca

SEQ ID NO: 17 - DNA, 375 bp, Artificial sequence
  Synthetic antibody
cagctggtggaggagtccggggggggcctggtcaagcctggggcgcccctgacactcacctgcacactctctggattctccttcagtagcggctactacatgtgctgggtccgccaggctccagggaaggggctggagtggatcgcatgcatttatggtgctgttagtgataggatttacttcgcgagctgggcgaaaggccgattcaccatgtccagaccctcgtcgaccacggtgactctgcaaatgaccagtctgacagccgcggacacggccacctatttctgtgcgagagatagttatagtggtagtacttattatagatcatactactttaacttgtggggcccaggcaccctcgtcaccgtctcttca

SEQ ID NO: 18 - DNA, 360 bp, Artificial sequence
  Synthetic antibody
cagtcagtgaaggagtccgggggaggcctggtccagcctgggggatccctgacactctcctgcaaagcctctggattcgacttcagtagttactacatgagctgggtccgccaggctccagggaaggggctggagtggatcgggatcatttatggtggtaaaggtaccacagactacgcgagctgggtgaatggccgattcaccatctccagcgacaacgcccagaacactgtggatcttcaaatgaacagtctgacagcggcggacacggccacctatttctgtgcgagagactatgttagtagtagtggttattatatggacttgtggggcccaggcaccctggtcaccgtctcttca

SEQ ID NO: 19 - DNA, 348 bp, Artificial sequence
  Synthetic antibody
cagcagctgaaggagtccgggggaggcctggtcaagcctggaggaaccctgacactcacctgcaaagcctctggaatcgacttcagtagcaatgtagtgtgctgggtccgccaggctccagggaaggggctggagtggatcggatgcattgatactgatgattttattggcacttactacgcgagttgggcgaaaggccgattcaccacctccaagacctcgtcgaccacggtgactcttcaaatgaccagtctgacagccgcggacacggccacctatttctgtgcgaggggagatgctggttactatgccttgtggggcccaggcaccctggtcaccgtctcctca

SEQ ID NO: 20 - DNA, 375 bp, Artificial sequence
  Synthetic antibody
cagtcggtggaggagtccgggggagacctggtcaagcctggggcatccctgacactcacctgcacagcttctggattctccttcagtagcagctactacatgtgctgggtccgccaggctccagggaaggggctggagtggatcgcatgcatttatgctggtagtagtggtagcacatggtacgcgagctgggcgaaaggccgattcaccatctccattacctcgtcgaccacggtgactctgcaaatgaccagtctgacagccgcggacacggccacctatttctgtgcgagagaccgttatggtggtggttcttattatataccttactactttgacttgtggggcccaggcaccctggtcaccgtctcttca

SEQ ID NO: 21 - DNA, 354 bp, Artificial sequence
  Synthetic antibody
cagtcggtggaggagtccgggggaggcctggtcaagcctgagggatccctgacactcacctgcacagcttctggattctccttcagttacaggtactacatgtgctgggtccgccaggctccagggaaggggctggagtggatcggatgcattgataatggtgatggcgacacatactacgcgagctgggtgaatggccgattcaccatctccaaagcctcgtcgaccacggtgactctgcaaatgaccagtctgacagccgcggacacggccacctatttctgtgcgagagggagtagtggttataatggctttaacttctggggcccaggcaccctggtcaccgtctcttca

SEQ ID NO: 22 - DNA, 363 bp, Artificial sequence
  Synthetic antibody
cagcagctgaaggagtccgggggagacctggtcaagcctggggcatccctgacactcacctgcacagcctctggattctccttcagtagcggctactacatgtgctgggtccgccaggctccagggaagggactggagtggatcgcatgcattggtgctagtagtggactcactaactacgcgagctgggcgaaaggccgattcaccatctccaaaacctcgtcgaccacggtgactctgcgaatgaccagtctgacagccgcggacacggccacctatttctgtgcgagagtctatcttggtggtgctgattatgagcaatttaatttgtggggtccaggcaccctcgtcaccgtctcttca

SEQ ID NO: 23 - DNA, 375 bp, Artificial sequence
  Synthetic antibody
caggagcagctggtggagtccgggggagacctggtcaagcctgagggatccctgacactcacctgcacagcctctggattctccttcagtgacaactactggatgtgctgggtccgccaggctccagggaaggggctggagtggatcggatgcatttatattggttttagtggtaccacttactacgcgagctgggcgaaaggccgattcaccatctccaaggcctcgtcgaccacggtgactctgcaaatgaccagtctgacaggcgcggacacggccacctatttctgtgcgagaggggttttttatgttggtagtggttattggtactacttgaacttgtggggcccaggcaccctggtcaccatctcttca

SEQ ID NO: 24 - DNA, 351 bp, Artificial sequence
  Synthetic antibody
caggagcagctggtggagtctggagggggtctggtcacgcctggaggaaccctgacactcacctgcacagcctctggattcgacttcagtagctactactacatgtgctgggtccgccaggctccagggaaggggctggagttgatcgcagcgattgatactggtagtggtgagacttactacgcgagctgggcgaacggccgattcaccatctccaaaacctcgtcgaccacggtgactctgcaaatgaccagtctgacagccgcggacacggccacctatttctgtgcgagagatccctattttggcactaacttatggggcccaggcaccctggtcaccgtctcttca

SEQ ID NO: 25 - DNA, 364 bp, Artificial sequence
  Synthetic antibody
caggagcagctggtggagtccgggggagacctggtcaagcctggggcatccctgacactcacctgcacagcctctggattctccttcagtagcagccactatatgtgctgggtccgccaggctccagggaaggggctggagtggatagcgtgtatttatcttggtgatgacagcacatactacgcgagctgggtgaatggccgattctccatctccaaaacctcgtcgaccacggtgactctgcaaatgaccagtctgacagccgcggacacggccacctatttctgtgcgagacatggatataatcttgctattcataattatattatatggggcccaggcaccctggtcactgtctcttcag

SEQ ID NO: 26 - DNA, 355 bp, Artificial sequence
  Synthetic antibody
cagtcagtgaaggagtccgggggagacctggtcaagcctggggcatccctgacactcacctgcacagcctctggattctccttcagtagcgactacgacatatactgggtccgccaggctccagggaaggggctggagtggatcgcatggattgatggtggtagtagtggtgagatttatgcgaactgggcgaaaggccgattcaccatctccaaaacctcgtcgaccacggtgactctgcaaatgaccagtctgacagccgcggacacggccgcctatttctgtgcgagatgtatttggagtcataattactatgacttgtggggcccaggcaccctggtcaccgtctcttcag

SEQ ID NO: 27 - DNA, 376 bp, Artificial sequence
  Synthetic antibody
caggagcagctggtggagtccgggggagacctggtcaagcctggggcatccctgacactcacctgcacagcctctggattctccttcagtagcagctactggatatgctgggtccgccaggctccagggaaggggctggagttaatcgcatgcatttatcctagtagtagtggtagcactgactacgcgagctgggcgaaaggccgattcaccatctccaaaacctcgtcgaccacggtgactctgcaaatgaccagtctgacagccgcggacacggccacctatttctgtgcgagagatattccttataatattcatggttatactaccggatttaacttgtggggcccaggcaccctggtcaccgtctcctcag

SEQ ID NO: 28 - DNA, 355 bp, Artificial sequence
  Synthetic antibody
cagtcggtggaggagtccgggggaggcctggtccagcctgggggatccctgacactcacctgcaaagcctctggattctccttcagtagcggctactggatgtgttgggtccgccaggctccagggaaggggctggaatggatcgcatgtattggtgctagttatggtggcactttctacgcgagctgggcgaaaggccgattcaccatctccaaaacctcgtcgaccacggtgactcttcaaatgaccagtctgacagccgcggacacggccacctatttctgtacgagagatgatggtggtcttgataatcctaatttgtggggcccaggcaccctggtcactgtctcttcag

SEQ ID NO: 29 - DNA, 370 bp, Artificial sequence
  Synthetic antibody
cagtcggtggaggagtccgggggagacctggtcaagcctggggcatccctgacactcacctgcacagcctctggattcaccatcagtagtgactactacatgtgctgggtccgccaggctccagggaaggggctggagtggatcggatgtatttatattggtagtggtactgatacttattacgcgagctgggcgaaaggccgattcaccatctccaaaacctcgtcgaccacggtgactctacaaatgaccagtctgacagccgcggacacggccacctacttctgtgcgagagataagggatggtctaatgcctggggtttctactttcagttgtggggcccaggcaccctggtcaccgtctcctcag

SEQ ID NO: 30 - DNA, 367 bp, Artificial sequence
  Synthetic antibody
caggagcagctggaggagtccgggggagacctggtcaagcctgagggatccctgacactcacctgcacagcctctggattctccttcagtagcagctactggatatgctgggtccgccaggctccagggaaggggctggagtggatcgcatgcatttatgctggtggtagtggtagcacttactacgcgagctgggcgaaaggccgattcaccatctccaaaacctcgtcgaccacggtgactctgcaaatgaccagtctgacagccgcggacgcggccacctatttctgtgcgagagatggtgttagtggtagtggttggttctttaacttgtggggcccaggcaccctggtcaccgtctcttcag

SEQ ID NO: 31 - DNA, 367 bp, Artificial sequence
  Synthetic antibody
cagtcggtggaggagtccgggggagacctggtcaagcctggggcatccctgacactcacctgcacagcctctggattctccttcagtaacaactactggatatgctggatccgccaggctccagggaaggggctggagtggatcgcatgcatttatgctggtgatactggtaccgcttactacgcgaactgggcgaaaggccgattcaccatctccaaaacctcgtcgaccacggtgactctacaaatgaccagtctgacagccgcggacacggccacatatttctgtgcgagagatggggatggtgttgggaatggtggtacttttgccttgtggggcccaggcaccctggtcaccgtctcctcag

SEQ ID NO: 32 - DNA, 367 bp, Artificial sequence
  Synthetic antibody
cagtcggtggaggagtccgggggaggcctggtccagcctgagggatccctgacactcacctgcacagcttctggattctccttcagtagcagctactacatgtgctgggtccgccaggctccagggaaggggctggagtggatcgcgtgcatttttactggtggtgttagcacgtactacgcgagctgggtgaatggccgattcaccatctccaaaacctcgtcgaccacggtgactctgcaaatgaccagtctgacagccgcggacacggccacctatttctgtgcgagaacctatactgctggttttactggtgataactactttaacttgtggggcccaggcaccctggtcaccgtctcttcag

SEQ ID NO: 33 - DNA, 379 bp, Artificial sequence
  Synthetic antibody
cagtcggtggaggagtccgggggagacctggtcaagcctggggcatccctgacactcacctgcacagcctctggattctccttcagtggcccctactggataagttgggtccgccaggctccagggagggggctggagtggatcgcaagtattggtgctcgtagtagtggtagcactcactacgcgagctgggcgaaaggccgattcaccatctccaaaaccgcgtcgaccacggtgactctgcaaatgaccagtctgacagccgcggacacggccacctatttctgtgcgagatcgtatcctgcttatgctggtgacggttatgccctatactactttaacttgtggggcccaggcaccctggtcaccgtctcttcag

SEQ ID NO: 34 - DNA, 333 bp, Artificial sequence
  Synthetic antibody
gcccttgtgatgacccagactccatcttccgcgtctgaacctgtgggaggcacagtcaccatcaagtgccaggccagtgagagcattgataatgatttagcctggtatcagcagaaaccagggcagcctcccaagctcctgatctaccaggcatccactctggcatctggggtcccatcgcgattcaaaggcagtggatctgggacagaattcactctcaccatcagcgacctggagtgtgccgatgctgccacttactactgtcaaagtaattattatagtggtagtagtagtagttatggtttcggcggagggaccgaggtggtcgtcaaa

SEQ ID NO: 35 - DNA, 333 bp, Artificial sequence
  Synthetic antibody
gcccttgtgatgacccagactccatcttccgcgtctgaacctgtgggaggcacagtcaccatcaagtgccaggccagtgagagcattagtagtagattagcctggtatcagcagaaaccagggcagcctcccaagctcctgatgtttgatgcatccgctctggcatctggggtctcctcgcggttcaaaggcaggggatctgggacacagttcactctcaccatcagcgacctggagtgtgccgatgctgccacttactactgtcaaagttattattatagtggtggggttcctagttatggtttcggcggagggaccaaggtggtcgtcgaa

SEQ ID NO: 36 - DNA, 333 bp, Artificial sequence
  Synthetic antibody
gccatcaaaatgacccagactccatcctccgtgtctgcagctgtgggaggcacagtcaccatcaattgccgggccagtgaggacattgaaagctatttagcctggtatcagcagaaaccagggcagcctcccaagctcctgatctatgatgcatccaatctggaaactggggtcccatcgcggttcaaaggcagtggatctgggacagagttcactctcaccatcagcggcgtgcagtgtgacgatggtgccacttattactgtcaaggtgcttggtatagtagtagtgttgatatgtatgctttcggcggagggaccgagctggagatccta

SEQ ID NO: 37 - DNA, 330 bp, Artificial sequence
  Synthetic antibody
gccatcaaaatgacccagactccatcctccgtgtctgcagctgtgggaggcacagtcaccatcaattgccaggccagtcagaacatttacagcaatttagcctggtatcagcagaacccagggcagcctcccaagctcctgatctattctgcatccactctggcatctggggtcccatcgcggttcaaaggcagtggatctgggacagagttcactctcaccatcagcgacctggactgtgccgatgctgccacttactactgtcaacaggattatagttggagtaatgttgataatgttttcggcggagggaccaagctggagatcaaa

SEQ ID NO: 38 - DNA, 327 bp, Artificial sequence
  Synthetic antibody
gatgtcgtgatgacccagactccagcctccgtggaggcagctgtgggaggcacagtcaccatcaagtgccaggccagtgaggacattgaaagttatttagcctggtatcagcagaaaccagggcagcgtcccagactcctgatctatgatgcatccgatctggcatctggggtcccatcgcggttcaaaggcagtggatctgggacagagttcactctcaccatcagcggcgtgcagtgtgccgatgctaccacttactactgtcaaggctattcttatggtagtagttatgttggtttcggcggagggaccgaggtggtcgtcaaa

SEQ ID NO: 39 - DNA, 330 bp, Artificial sequence
  Synthetic antibody
gcccaagtgctgacccagactccagcctctgtggaggtagctgtgggaggcacagtcaccatcaagtgccaggccagtcagagcatttacagctacttatcctggtatcagcagaaaccagggcagcctcccaagctcctgatgtatggtgcatccactctggcatctggggtctcatcgcggttcgaaggcagtggatctgggacacagttcactctcaccatcagcggcgtggagtgtgccgatgctgccacttactactgtcaacagggttatactagttataatattgacaatgctttcggcggagggaccgaggtggtcgtcaaa

SEQ ID NO: 40 - DNA, 339 bp, Artificial sequence
  Synthetic antibody
gatgtcgtgatgacccagactccagcctccgtgtctgcagctgtgggaggcacagtcaccatcaagtgccaggccagtgaggacatttatagcttattggcctggtatcagcagaaaccagggcagcctcccaagctcctgctctatctggcatctactctggcatctggggtcccatcgcggttcaaaggcagtggatctgggacagagtacactctcaccatcagtgacctggagtgtgccgatgctgccacttactactgtcaaaactatcattatagtagtactactagttatgcccccaatcctttcggcggagggaccgagctggagatccta

SEQ ID NO: 41 - DNA, 309 bp, Artificial sequence
  Synthetic antibody
gatgtcgtgatgacccagactccatcctctgtgtctgcagctgtgggaggcacagtcaccatcaattgccaggccagtcagagtgttagcaacctcttagcctggtatcagcagaaaccagggcgccctcccgcgctcctgatttatggtgcatccaatctggaatctggggtcccatcgcgtttccgtggcagtggatctgggacacagttcactctcaccatcagtggcatgaaggctgaagatgctgccacttattactgtcaaagtggttattactttggagctggcaccaagctggagatcaaa

SEQ ID NO: 42 - DNA, 336 bp, Artificial sequence
  Synthetic antibody
gccatcaaaatgacccagactccagcctccgtgtctgaacctgtgggaggcacagtcaccatcaagtgccaggccagtcagagcattagcagttggttatcctggtatcagcagaaaccagggcagcctcccaagcgcctgatctacaaggcatccactctggcatctggggtcccatcgcggttcaaaggcagtggatctgggacagagttcactctcaccatcagcgacctggagtgtgccgatgctgccacttactactgtcaaagcaattatggtagttatagtagtagttatggatatgctttcggcggagggaccgaggtggtcgtcaaa

SEQ ID NO: 43 - DNA, 333 bp, Artificial sequence
  Synthetic antibody
gaccctatgctgacccagactccatcctccgtgtctgcagctgtgggaggcacagtcagcatcagttgccagtccagtcagagtgtttatgataacgactggttatcctggtatcagcagaaaccagggcagcctcccaagctcctgatctatgatgcatccaaattggcatctggggtcccatcgcggttcaaaggcagtggatctgggacacagttcactctcaccatcagcgacgtgcagtgtgacgctgctgccacttactactgtgcaggcgactatattaatactattgataatgctttcggcggagggaccgaggtggtcgtcaaa

SEQ ID NO: 44 - DNA, 327 bp, Artificial sequence
  Synthetic antibody
gatgtcgtgatgacccagactccagcctccgtggaggcagctgtgggaggcacagtcaccatcaagtgccaggccagtgaggacattgaaagctatttagcctggtatcagcagaaaccagggcagcctcccaagctcctgatctatgatgcatccgatctggcatctggggtcccatcgcggttcaaaggcagtggatctgggaaacagttcactctcaccatcagcggcgtgcggtgtgccgatgctgccacttactactgtcaaggctattattatggtgctatttatgttgctttcggcggagggaccaagctggagatcaaa

SEQ ID NO: 45 - DNA, 330 bp, Artificial sequence
  Synthetic antibody
gccatcaaaatgacccagactccagcctctgtggaggtagctgtgggaggcacagtcaccatcaagtgccaggccagtcagagcattggtatctacttagcctggtatcagcagaaaccagggcagcctcccaagctcctgatctatgatgcatccgatctggcatctggagtcccatcgcggttcagaggcagtggatctgggacacagttcactctcaccatcagcggcgtggagtgtgccgatgctgccacttactactgtcaacagggtgatactatttataatgttgataatactttcggcggagggaccaaggtggtcgtcgaa

SEQ ID NO: 46 - DNA, 327 bp, Artificial sequence
  Synthetic antibody
gaccctatgctgacccagactccagcctccgtgtctgaacctgtgggaggcacagtcaccatcaagtgccaggccagtcagagcattagcactgcattagcctggtatcagcagaaaccagggcagcctcccaagctcctgatctacagggcatccattctggcatctggggtcccatcgcggttcaaaggcagtggatctgggacagagttcactctcaccatcagcgacctggagtgtgccgatgctgccacttactactgtcaaagctattatgatagtagtgctggtaatactttcggcggagggaccaaggtggaaatcaaa

SEQ ID NO: 47 - DNA, 330 bp, Artificial sequence
  Synthetic antibody
gcccaagtgctgacccagactccagcctctgtggaggtagctgtgggaggcacagtcaccatcaagtgccaggccagtcagaccattagcaacctcttagcctggtatcagcagaaaccagggcagcctcccaaactcctaatctatgatgcatccactctggcatatggggtctcatcgcgattcagcggcagtggatctgggacacagttcactctcaccatcagcggcgtgcagtgtgccgatgctgccacttattactgtcaacagggttggagtagtagtaatgttgataatgtttttggcggagggaccaagctggagatcaaa

SEQ ID NO: 48 - DNA, 330 bp, Artificial sequence
  Synthetic antibody
gccatcaaaatgacccagactccagcctccgtgtctgaacctgtgggaggcacggtcaccatcaagtgccaggccaatgagagcattagcagttggtgtgcctggtatcagcagaaaccagggcagcctcccaagctcctgatctaccaggcatccaaactggcatctggggtcccatcgcggttcaaaggcagtggatctgggacacagttcactctcactatcagcgacctggagtgtgccgatgctgccacttactactgtcaatctacttattataatgagaatgttggaaatgcgttcggcggagggaccgaggtggtcgtcaaa

SEQ ID NO: 49 - DNA, 327 bp, Artificial sequence
  Synthetic antibody
gcccaagtgctgacccagactccagcctccgtggaggcagctgtgggaggcacagtcaccataaagtgccaggccagtgagagcattggcaatgcattagcctggtatcaacagaaaccagggcagcctcccaagctcctgatctatgttgcatcctatctggcatctggggtctcatcgcggttcaaaggcagtagatctgggacagagtacactctcaccatcagcgacctggagtgtgtcgatgctgccacttactactgtcaatgtacttggtatggtaatgtctatcttgctttcggcggagggaccgaggtggtcgtcaaa

SEQ ID NO: 50 - DNA, 333 bp, Artificial sequence
  Synthetic antibody
gaccctgtgctgacccagactccagcctccgtgtctgcagctgtgggaggcacagtcaccatcagttgccagtccaatgaaaatatttatgtagacaaatacttatcctggtatcaacagaaaccagggcagcctcccaaaggcctgatctattctgcatccactctggcatctggggtcccatcgcgcttcagaggcagtggatctgggacacagttcactctcaccatcagcgacgtgcagtgtgacgatgctgccacttactactgtgcagggagttttagtagtagtgatagttgtgctttcggcggagggaccgaggtggtcgtcaaa

SEQ ID NO: 51 - DNA, 333 bp, Artificial sequence
  Synthetic antibody
gcccttgtgatgacccagactccagcctccgtgtctgcagctgtgggaggcacagttaccatcaagtgccaggccagtgaggacatttatagcttattggcctggtatcagcagaaaccagggcagcctcccaagctcctgatctatgatgcatccgatctggcatctggggtcccatcgcggttcagcggcagtggatctgggacagagtacactctcaccatcagcgacctggagtgtgacgatgctgccacttactactgtcaaggcggttattatagtggtagtcctactgataattctttcggcggagggaccaaggtggtcgtcgaa

SEQ ID NO: 52 - DNA, 333 bp, Artificial sequence
  Synthetic antibody
gatgtcgtgatgacccagactccagcctccgtgtctgaacctgtgggaggcacagtcaccatcaagtgccaggccagtcagagcattggtagctatttagcctggtatcagcagaaaccagggcagcctcccaagctcctgatctattatgcctccactctggcatctggggtctcatcgcggttcaaaggcagtagatctgggacagagtacacgctcaccatcagcgacctggagtgtgccgatgctgccacttactactgtcaatgttcttattttggtagtggtgctgtcgatagtcctttcggcggagggaccaaggtggtcgtcgaa

SEQ ID NO: 53 - DNA, 333 bp, Artificial sequence
  Synthetic antibody
gcccttgtgatgacccagactccatcccctgtgtctgcagctgtgggaggcacagtcaccatcaattgccaggccagtcagaacatttatgtcgacagctacttagcctggtatcagcagaaatcagggcagcctcccaaactcctgatctattatgcatccagtctggcatctggggtcccatcgcggttcagcggcagtggatctgggacacagttcactctcaccatcagcgacctggagtgtgacgatgctgccacttactactgtgcaggcggttatagtgctgattcggataatgctttcggcggagggaccgaggtggtcgtcaaa

SEQ ID NO: 54 - DNA, 330 bp, Artificial sequence
  Synthetic antibody
gcccttgtgatgacccagactccagcctccgtgtctgcagctgtggcaggcacagtcaccatcaagtgccaggccagtcagagcattggtagctacttagcctggtatcagcagaaaccagggcagcctcccaagctcctgatctatgctacatccattctggcatctggggtctcatcgcggttaaaaggcagtagatctgggacagagtacactctcaccatcagcgacctggagtgtgccgatgctgccacttactactgtcaatgtggttattatgatagtaattatgggggtgctttcggcggagggaccgaggtggtcgtcaaa

SEQ ID NO: 55 - DNA, 324 bp, Artificial sequence
  Synthetic antibody
gcccaagtgctgacccagactccatcttccgcgtctgaacctgtgggaggcacagtcaccatcaagtgccaggccggtgagagcgttagtagtagagtagcctggtatcagcagaaaccagggcagcctcccaagctcctgatctttgatgcatccgctctggcatctggggtctcatcgcggttcacaggcagtggatctgggacacagttcactctcaccatcagcgacctggagtgtgccgatgctgccacttactactgtcaaaactattatggtattagtggtgtcgctttcggcggagggaccgaggtggtcgtcaaa

SEQ ID NO: 56 - DNA, 327 bp, Artificial sequence
  Synthetic antibody
gatgtcgtgatgacccagactccatcctccgtgtctgcagctgtgggaggcacagtcaccatcaagtgccaggccagtcagagcattgctagttacttagcctggtatcagcagaaaccagggcagcctcccaagctcctgatctacagggcatccactctggcatctggggtcccatcgcggttcaaaggcagtggatctgggacagagtacactctcaccattagcgacctggagtgtgccgatgctgccacttactactgtcaaactacttatgttggtggtagtggcaatgctttcggcggagggaccaaggtggaaatcaaa

SEQ ID NO: 57 - DNA, 333 bp, Artificial sequence
  Synthetic antibody
gaccctatgctgacccagactccatctcccgtgtctgcagctgtgggaggcacagtcaccatcacttgccaggccagtcagagtgtttataacaacaactacttatcctggtttcagcagaaaccagggcagcctcccaagctcctgatctacaaggcttccattctggcatctggggtcccatcgcggttcaaaggcagtggatctgggacacagttcactctcaccatcagcgacctgcagtgtgacgatgctgccacttactactgtctaggcggttatagtagtcctagtgatgatgctttcggcggagggaccaaggtggtcgtcgaa

SEQ ID NO: 58 - DNA, 337 bp, Artificial sequence
  Synthetic antibody
gatgtcgtgatgacccagactccatcctccgtggaggcaggtgtgggaggcacagtcaccatcaagtgccaggccagtgagagtagtagtcgtcacttatcctggtatcagcagaaaccagggcagcgtcccaagctcctgatctactgggcatccactctggcatctggggtcccatcgcggttccaaggcagtggatctgggacagagttcactctcaccatcagcgacctggagtgtgccgatgctgccacttactactgtcaaagctattatgggcttagtaataatggttggagatatgctttcggcggagggaccgaggtggtcgtcaaag

SEQ ID NO: 59 - DNA, 331 bp, Artificial sequence
  Synthetic antibody
gccatcaaaatgacccagactccatcctccgtgtctgcagctgtgggaggcacagtcaccatcaattgccaggccagtgaggacatcgaaaactatttagcctggtatcaacagaaaccagggcagcctcccaagctcctgatctacagggcatccactctggcatctggggtctcatcgcggttcaaaggcagtggatctgggacagaattcactctcaccatcagcgacctggagtgtgccgatgctgccacttactggtgtcaacagggttatagtggtagtaatgttgataattttttcggcggagggaccaaggtggtcgtcgaag

SEQ ID NO: 60 - DNA, 331 bp, Artificial sequence
  Synthetic antibody
gcccttgtgatgacccagactccagcctccgtggaggcagctgtgggaggcacagtcaccatcaagtgccaggccagtcagagcattggtagtaatttagcctggtatcagcagaaaccagggcagcctcccaagctcctgatctattctgcatccactctggcatctggggtctcatcgcggttcaaaggcagtagatctgggacagagttcactctcaccatcagcgacctggagtgtgccgatgctgccacttactactgtcaatttacttattatggtagtagtgatgtcgatgttttcggcggagggaccaaggtggtcgtcgaag

SEQ ID NO: 61 - DNA, 337 bp, Artificial sequence
  Synthetic antibody
gaccctgtgctgacccagactccagcctccgtggaggcagctgtgggaggcacagtcaccatcaagtgccaggccagtcagagcattggtagtattttagcctggtatcagcagaaaccagggcagcctccccacctcctgatctatgctgcagccaagctggcatctggggtcccatcgcggttcaaaggcagtggatctgggacacagttcactctcaccatcagcggcgtggagtgtgacgatgctgccacttactactgtcaaagctattattatagtgttaccactagttattcgaatagtttcggcggagggaccgaggtggtcgtcaaag

SEQ ID NO: 62 - DNA, 331 bp, Artificial sequence
  Synthetic antibody
gaccctgtgctgacccagactccagcctctgtggaggtacctgtgggaggcacagtcaccatcaattgccaggccagtcagagcattggtaaatacttaaactggtatcagcagaaaccagggcagcctcccaaactcctgatctattcttcgtccagtctggcatctggggtctcatcgcggttcaaaggcagtggatttgggacacagttcactctcaccatcagcggcgtgcagtgtgccgatgctgccacttactactgtcaacagggttatagttatgttgatcttgaaaatggtttcggcggagggaccgagctggagatcctag

SEQ ID NO: 63 - DNA, 337 bp, Artificial sequence
  Synthetic antibody
gatgtcgtgatgacccagactccagcctccgtgtctgaacctgtgggaggcacagtcaccatcaagtgccaggccagtcagagcattagtagctacttaacctggtatcaacagaaaccagggcagcctcccaagctcctgatctacaaggcatccactctggcatctggggtctcatcgcggttcaagggcagtggatctgggacagagttcactctcaccatcagcgacctggagtgtgccgatgctgccacttactactgtcaaagtagttattatggtcctagtgatgttgatttttctcctttcggcggagggactgaggtggaaatcaaag

SEQ ID NO: 64 - DNA, 331 bp, Artificial sequence
  Synthetic antibody
gcccttgtgatgacccagactccagcctccgtggaggcagctgtgggaggcacggtcaccatcaggtgccaggccagtgagaggattggcaatgcattagcctggtatcagcagaaaccagggcagcctcccaagctcctgatctaccagacatccaaactgacatctggggtcccatcgcggttcaaaggcagtggatctgggacagaacacactctcaccatcagcgacctggagtgtgccgatgctgccacttactactgtcaaagctattattatactggcagtattagttatgaattcggcggagggaccaaggtggtcgtcgaag

SEQ ID NO: 65 - DNA, 337 bp, Artificial sequence
  Synthetic antibody
gcccaagtgctgacccagactccagcctccgtggaggcagctgtgggaggcacagtcaccatcaagtgccaggccagtcagagtattagtagttacttagcctggtatcagcagaaaccagggcagcctcccaagctcctgatctacagggcatccactctggcatctggggtcccatcgcggttcaaaggcagtggatctgggacagagttcactctcaccatcagcgacctggagtgtgccgatgctgccacttactactgtcaaagtgattattatagtagtagtagcaataatggttttgttttcggcggagggaccgaggtggtcgtcaaag

SEQ ID NO: 66 - DNA, 334 bp, Artificial sequence
  Synthetic antibody
gatgtcgtgatgacccagactccagcctccgtgtctgcagctgtgggaggcacagtcaccatcaagtgccaggccagtgaagacatttatagtttattggcctggtatcagcagaaaccagggcagcctcccaagttcctgatctatgatgcatccgatctggcatctggggtcccatcgcggttcagcggcagtggatctgggacagagtacactctcaccatcaacgacctgaagtgtgacgatgctgccacttactactgtcaaggcggttattatagtggtgatggtactgatatacctttcggcggagggaccgagctggagatcctag

SEQ ID NO: 67 - protein, 114 aa, Artificial sequence
  Synthetic antibody
EQLVEESGGGLVQPGGSLTLSCTASGWDLSDTYWICWVRQAPGKGLEWIGCIYNGNGDTYYATWAKGRFTISKASTTVDLKMTSLTAADTATYFCARGVAADLWGPGTLVTVSS

SEQ ID NO: 68 - protein, 122 aa, Artificial sequence
  Synthetic antibody
EQLKEESGGGLVQPEGSLTLTCTASGFSFSSGYYMCWVRQAPGKGLEWSACIGASSGLTYFASWAKGRFTISKTSSTTVTLRMTSLTAADTATYFCARVYLGGADYERFNLWGPGTLVTVSS

SEQ ID NO: 69 - protein, 120 aa, Artificial sequence
  Synthetic antibody
QKQLVETGGGLVQPGGSLTLSCKVSEFDFSSYYIHWVRQAPGKGLEWIGIIYAGKGDTDYASWVNGRFTISSDNAQNTVDLQMNSLTAADTATYFCARGWLGSDYYFNFWGPGTLVTVSS

SEQ ID NO: 70 - protein, 117 aa, Artificial sequence
  Synthetic antibody
QEQLVESGGGLVQPEGSLTLTCKASGLDFSSSYWICWVRQAPGKGLEWIGYINTSSGSTWYASWAKGRFTISKTSSTTVTLQMTSLTAADTAIYFCAKYINSYFGLWGPGTLVTVSS

SEQ ID NO: 71 - protein, 116 aa, Artificial sequence
  Synthetic antibody
QQLVESGGDLVKPEGSLTLTCTASGFTLTTYWVCWVRQAPGKGLEWIACIDAGSITYYTTWARGRFTVSRTSSTTVTLQMTSLTAADTATYFCARDAHYTYALVLWGPGTLVTVSS

SEQ ID NO: 72 - protein, 121 aa, Artificial sequence
  Synthetic antibody
QQLKESGGDLVKPGASLTLTCTASGFSFSNNNYMCWVRQAPGKGLEWVACIYTGDAYTYFASWAKGRFTISKTSSTTVTLQMTSLTAADTATYFCARGYSGYSGYGYSDLWGPGTLVTVSS

SEQ ID NO: 73 - protein, 119 aa, Artificial sequence
  Synthetic antibody
QSVEESGGDLVKPGASLTLTCTASGFSFSSSYWMCWVRQAPGKGLEWIACIYTGDGANYYASWAKGRFTISKASSTTVTLQMTSLTAADTATYFCARSAYGGYGYFNFWGPGTLVTVSS

SEQ ID NO: 74 - protein, 121 aa, Artificial sequence
  Synthetic antibody
QSVKESGGDLVKPGASLTLTCTASGFSFSGAYWICWVRQAPGKGLEVIACIYVISSSNTYYASWAKGRFTCSKTSSTTVDLKMTSLTAADTATYFCARYSCSSAFCLFNLWGPGTLVTVSS

SEQ ID NO: 75 - protein, 120 aa, Artificial sequence
  Synthetic antibody
QKQLVESGGGLVQPEGSLTLTCKASGFDFSSNAMCWVRQAPGKGLELLACFDYISDSIWYASWVNGRFTISKTSSTTVTLQMTSLTAADTATYFCARDNVGDGYTTFDLWGPGTLVTVSS

SEQ ID NO: 76 - protein, 117 aa, Artificial sequence
  Synthetic antibody
QSVKESGGGLVQPEGSLTLTCTASGFDFSSNAMCWVRQAPGKGPEWIACIYNGDASTYYASWAKGRSTISKTSSTTVTLQMTSLTAADTATYFCARCFNRFYRGDLWGPGTLVTVSS

SEQ ID NO: 77-78 - skipped (2 entries)

SEQ ID NO: 79 - protein, 120 aa, Artificial sequence
  Synthetic antibody
KQLVEESGGDLVRPEGSLTLTCKGSGFDFSRNAMCWVRQAPGKGLEWIACIYVGSGGGTYYATWAKGRFTISKTSSTTVTLQMTSLTAADTATYFCAGSPYSGYGYFNLWGPGTLVTVSS

SEQ ID NO: 80 - protein, 120 aa, Artificial sequence
  Synthetic antibody
QEQLVEYGGDLVQPEGSLTLTCTASGFTISSTFYMGWVRQAPGKGLEWIGDMHVGSATTTYYASWAKGRFTISKPSSTTVTLQMTSLTAADTATYFCARYVNSGYYMDSWGPGTLVTVSS

SEQ ID NO: 81 - protein, 122 aa, Artificial sequence
  Synthetic antibody
QSVEESGGDLVKPGASLTLTCTASGFSFSSSHYMWWVRQAPGKGLEWIGCIHISSGVTWYASWAKGRFTISKTSSTTVTLQMTSLTAADTATYFCARDDRYGHGGYPYTDLWGPGTLVTVSS

SEQ ID NO: 82 - protein, 119 aa, Artificial sequence
  Synthetic antibody
QSVKESGGGLVQPEGSLTLTCTASGFSFSSSYYMCWVRQAPGKGLEWIACIYNGDGSTYYASWAKGRFTISKTSSTTVTLQMTSLTAADTATYFCARDADYAGVVLNLWGPGTLVTVSS

SEQ ID NO: 83 - protein, 125 aa, Artificial sequence
  Synthetic antibody
QLVEESGGGLVKPGAPLTLTCTLSGFSFSSGYYMCWVRQAPGKGLEWIACIYGAVSDRIYFASWAKGRFTMSRPSSTTVTLQMTSLTAADTATYFCARDSYSGSTYYRSYYFNLWGPGTLVTVSS

SEQ ID NO: 84 - protein, 120 aa, Artificial sequence
  Synthetic antibody
QSVKESGGGLVQPGGSLTLSCKASGFDFSSYYMSWVRQAPGKGLEWIGIIYGGKGTTDYASWVNGRFTISSDNAQNTVDLQMNSLTAADTATYFCARDYVSSSGYYMDLWGPGTLVTVSS

SEQ ID NO: 85 - protein, 116 aa, Artificial sequence
  Synthetic antibody
QQLKESGGGLVKPGGTLTLTCKASGIDFSSNVVCWVRQAPGKGLEWIGCIDTDDFIGTYYASWAKGRFTTSKTSSTTVTLQMTSLTAADTATYFCARGDAGYYALWGPGTLVTVSS

SEQ ID NO: 86 - protein, 125 aa, Artificial sequence
  Synthetic antibody
QSVEESGGDLVKPGASLTLTCTASGFSFSSSYYMCWVRQAPGKGLEWIACIYAGSSGSTWYASWAKGRFTISITSSTTVTLQMTSLTAADTATYFCARDRYGGGSYYIPYYFDLWGPGTLVTVSS

SEQ ID NO: 87 - protein, 118 aa, Artificial sequence
  Synthetic antibody
QSVEESGGGLVKPEGSLTLTCTASGFSFSYRYYMCWVRQAPGKGLEWIGCIDNGDGDTYYASWVNGRFTISKASSTTVTLQMTSLTAADTATYFCARGSSGYNGFNFWGPGTLVTVSS

SEQ ID NO: 88 - protein, 121 aa, Artificial sequence
  Synthetic antibody
QQLKESGGDLVKPGASLTLTCTASGFSFSSGYYMCWVRQAPGKGLEWIACIGASSGLTNYASWAKGRFTISKTSSTTVTLRMTSLTAADTATYFCARVYLGGADYEQFNLWGPGTLVTVSS

SEQ ID NO: 89 - protein, 125 aa, Artificial sequence
  Synthetic antibody
QEQLVESGGDLVKPEGSLTLTCTASGFSFSDNYWMCWVRQAPGKGLEWIGCIYIGFSGTTYYASWAKGRFTISKASSTTVTLQMTSLTGADTATYFCARGVFYVGSGYWYYLNLWGPGTLVTISS

SEQ ID NO: 90 - protein, 117 aa, Artificial sequence
  Synthetic antibody
QEQLVESGGGLVTPGGTLTLTCTASGFDFSSYYYMCWVRQAPGKGLELIAAIDTGSGETYYASWANGRFTISKTSSTTVTLQMTSLTAADTATYFCARDPYFGTNLWGPGTLVTVSS

SEQ ID NO: 91 - protein, 121 aa, Artificial sequence
  Synthetic antibody
QEQLVESGGDLVKPGASLTLTCTASGFSFSSSHYMCWVRQAPGKGLEWIACIYLGDDSTYYASWVNGRFSISKTSSTTVTLQMTSLTAADTATYFCARHGYNLAIHNYIIWGPGTLVTVSS

SEQ ID NO: 92 - protein, 118 aa, Artificial sequence
  Synthetic antibody
QSVKESGGDLVKPGASLTLTCTASGFSFSSDYDIYWVRQAPGKGLEWIAWIDGGSSGEIYANWAKGRFTISKTSSTTVTLQMTSLTAADTAAYFCARCIWSHNYYDLWGPGTLVTVSS

SEQ ID NO: 93 - protein, 125 aa, Artificial sequence
  Synthetic antibody
QEQLVESGGDLVKPGASLTLTCTASGFSFSSSYWICWVRQAPGKGLELIACIYPSSSGSTDYASWAKGRFTISKTSSTTVTLQMTSLTAADTATYFCARDIPYNIHGYTTGFNLWGPGTLVTVSS

SEQ ID NO: 94 - protein, 118 aa, Artificial sequence
  Synthetic antibody
QSVEESGGGLVQPGGSLTLTCKASGFSFSSGYWMCWVRQAPGKGLEWIACIGASYGGTFYASWAKGRFTISKTSSTTVTLQMTSLTAADTATYFCTRDDGGLDNPNLWGPGTLVTVSS

SEQ ID NO: 95 - protein, 123 aa, Artificial sequence
  Synthetic antibody
QSVEESGGDLVKPGASLTLTCTASGFTISSDYYMCWVRQAPGKGLEWIGCIYIGSGTDTYYASWAKGRFTISKTSSTTVTLQMTSLTAADTATYFCARDKGWSNAWGFYFQLWGPGTLVTVSS

SEQ ID NO: 96 - protein, 122 aa, Artificial sequence
  Synthetic antibody
QEQLEESGGDLVKPEGSLTLTCTASGFSFSSSYWICWVRQAPGKGLEWIACIYAGGSGSTYYASWAKGRFTISKTSSTTVTLQMTSLTAADAATYFCARDGVSGSGWFFNLWGPGTLVTVSS

SEQ ID NO: 97 - protein, 122 aa, Artificial sequence
  Synthetic antibody
QSVEESGGDLVKPGASLTLTCTASGFSFSNNYWICWIRQAPGKGLEWIACIYAGDTGTAYYANWAKGRFTISKTSSTTVTLQMTSLTAADTATYFCARDGDGVGNGGTFALWGPGTLVTVSS

SEQ ID NO: 98 - protein, 122 aa, Artificial sequence
  Synthetic antibody
QSVEESGGGLVQPEGSLTLTCTASGFSFSSSYYMCWVRQAPGKGLEWIACIFTGGVSTYYASWVNGRFTISKTSSTTVTLQMTSLTAADTATYFCARTYTAGFTGDNYFNLWGPGTLVTVSS

SEQ ID NO: 99 - protein, 126 aa, Artificial sequence
  Synthetic antibody
QSVEESGGDLVKPGASLTLTCTASGFSFSGPYWISWVRQAPGRGLEWIASIGARSSGSTHYASWAKGRFTISKTASTTVTLQMTSLTAADTATYFCARSYPAYAGDGYALYYFNLWGPGTLVTVSS

SEQ ID NO: 100 - protein, 111 aa, Artificial sequence
  Synthetic antibody
ALVMTQTPSSASEPVGGTVTIKCQASESIDNDLAWYQQKPGQPPKLLIYQASTLASGVPSRFKGSGSGTEFTLTISDLECADAATYYCQSNYYSGSSSSYGFGGGTEVVVK

SEQ ID NO: 101 - protein, 111 aa, Artificial sequence
  Synthetic antibody
ALVMTQTPSSASEPVGGTVTIKCQASESISSRLAWYQQKPGQPPKLLMFDASALASGVSSRFKGRGSGTQFTLTISDLECADAATYYCQSYYYSGGVPSYGFGGGTKVVVE

SEQ ID NO: 102 - protein, 111 aa, Artificial sequence
  Synthetic antibody
AIKMTQTPSSVSAAVGGTVTINCRASEDIESYLAWYQQKPGQPPKLLIYDASNLETGVPSRFKGSGSGTEFTLTISGVQCDDGATYYCQGAWYSSSVDMYAFGGGTELEIL

SEQ ID NO: 103 - protein, 110 aa, Artificial sequence
  Synthetic antibody
AIKMTQTPSSVSAAVGGTVTINCQASQNIYSNLAWYQQNPGQPPKLLIYSASTLASGVPSRFKGSGSGTEFTLTISDLDCADAATYYCQQDYSWSNVDNVFGGGTKLEIK

SEQ ID NO: 104 - protein, 109 aa, Artificial sequence
  Synthetic antibody
DVVMTQTPASVEAAVGGTVTIKCQASEDIESYLAWYQQKPGQRPRLLIYDASDLASGVPSRFKGSGSGTEFTLTISGVQCADATTYYCQGYSYGSSYVGFGGGTEVVVK

SEQ ID NO: 105 - protein, 110 aa, Artificial sequence
  Synthetic antibody
AQVLTQTPASVEVAVGGTVTIKCQASQSIYSYLSWYQQKPGQPPKLLMYGASTLASGVSSRFEGSGSGTQFTLTISGVECADAATYYCQQGYTSYNIDNAFGGGTEVVVK

SEQ ID NO: 106 - protein, 113 aa, Artificial sequence
  Synthetic antibody
DVVMTQTPASVSAAVGGTVTIKCQASEDIYSLLAWYQQKPGQPPKLLLYLASTLASGVPSRFKGSGSGTEYTLTISDLECADAATYYCQNYHYSSTTSYAPNPFGGGTELEIL

SEQ ID NO: 107 - protein, 103 aa, Artificial sequence
  Synthetic antibody
DVVMTQTPSSVSAAVGGTVTINCQASQSVSNLLAWYQQKPGRPPALLIYGASNLESGVPSRFRGSGSGTQFTLTISGMKAEDAATYYCQSGYYFGAGTKLEIK

SEQ ID NO: 108 - protein, 112 aa, Artificial sequence
  Synthetic antibody
AIKMTQTPASVSEPVGGTVTIKCQASQSISSWLSWYQQKPGQPPKRLIYKASTLASGVPSRFKGSGSGTEFTLTISDLECADAATYYCQSNYGSYSSSYGYAFGGGTEVVVK

SEQ ID NO: 109 - protein, 111 aa, Artificial sequence
  Synthetic antibody
DPMLTQTPSSVSAAVGGTVSISCQSSQSVYDNDWLSWYQQKPGQPPKLLIYDASKLASGVPSRFKGSGSGTQFTLTISDVQCDAAATYYCAGDYINTIDNAFGGGTEVVVK

SEQ ID NO: 110 - protein, 109 aa, Artificial sequence
  Synthetic antibody
DVVMTQTPASVEAAVGGTVTIKCQASEDIESYLAWYQQKPGQPPKLLIYDASDLASGVPSRFKGSGSGKQFTLTISGVRCADAATYYCQGYYYGAIYVAFGGGTKLEIK

SEQ ID NO: 111 - protein, 110 aa, Artificial sequence
  Synthetic antibody
AIKMTQTPASVEVAVGGTVTIKCQASQSIGIYLAWYQQKPGQPPKLLIYDASDLASGVPSRFRGSGSGTQFTLTISGVECADAATYYCQQGDTIYNVDNTFGGGTKVVVE

SEQ ID NO: 112 - protein, 109 aa, Artificial sequence
  Synthetic antibody
DPMLTQTPASVSEPVGGTVTIKCQASQSISTALAWYQQKPGQPPKLLIYRASILASGVPSRFKGSGSGTEFTLTISDLECADAATYYCQSYYDSSAGNTFGGGTKVEIK

SEQ ID NO: 113 - protein, 110 aa, Artificial sequence
  Synthetic antibody
AQVLTQTPASVEVAVGGTVTIKCQASQTISNLLAWYQQKPGQPPKLLIYDASTLAYGVSSRFSGSGSGTQFTLTISGVQCADAATYYCQQGWSSSNVDNVFGGGTKLEIK

SEQ ID NO: 114 - protein, 110 aa, Artificial sequence
  Synthetic antibody
AIKMTQTPASVSEPVGGTVTIKCQANESISSWCAWYQQKPGQPPKLLIYQASKLASGVPSRFKGSGSGTQFTLTISDLECADAATYYCQSTYYNENVGNAFGGGTEVVVK

SEQ ID NO: 115 - protein, 109 aa, Artificial sequence
  Synthetic antibody
AQVLTQTPASVEAAVGGTVTIKCQASESIGNALAWYQQKPGQPPKLLIYVASYLASGVSSRFKGSRSGTEYTLTISDLECVDAATYYCQCTWYGNVYLAFGGGTEVVVK

SEQ ID NO: 116 - protein, 111 aa, Artificial sequence
  Synthetic antibody
DPVLTQTPASVSAAVGGTVTISCQSNENIYVDKYLSWYQQKPGQPPKGLIYSASTLASGVPSRFRGSGSGTQFTLTISDVQCDDAATYYCAGSFSSSDSCAFGGGTEVVVK

SEQ ID NO: 117 - protein, 111 aa, Artificial sequence
  Synthetic antibody
ALVMTQTPASVSAAVGGTVTIKCQASEDIYSLLAWYQQKPGQPPKLLIYDASDLASGVPSRFSGSGSGTEYTLTISDLECDDAATYYCQGGYYSGSPTDNSFGGGTKVVVE

SEQ ID NO: 118 - protein, 111 aa, Artificial sequence
  Synthetic antibody
DVVMTQTPASVSEPVGGTVTIKCQASQSIGSYLAWYQQKPGQPPKLLIYYASTLASGVSSRFKGSRSGTEYTLTISDLECADAATYYCQCSYFGSGAVDSPFGGGTKVVVE

SEQ ID NO: 119 - protein, 111 aa, Artificial sequence
  Synthetic antibody
ALVMTQTPSPVSAAVGGTVTINCQASQNIYVDSYLAWYQQKSGQPPKLLIYYASSLASGVPSRFSGSGSGTQFTLTISDLECDDAATYYCAGGYSADSDNAFGGGTEVVVK

SEQ ID NO: 120 - protein, 110 aa, Artificial sequence
  Synthetic antibody
ALVMTQTPASVSAAVAGTVTIKCQASQSIGSYLAWYQQKPGQPPKLLIYATSILASGVSSRLKGSRSGTEYTLTISDLECADAATYYCQCGYYDSNYGGAFGGGTEVVVK

SEQ ID NO: 121 - protein, 108 aa, Artificial sequence
  Synthetic antibody
AQVLTQTPSSASEPVGGTVTIKCQAGESVSSRVAWYQQKPGQPPKLLIFDASALASGVSSRFTGSGSGTQFTLTISDLECADAATYYCQNYYGISGVAFGGGTEVVVK

SEQ ID NO: 122 - protein, 109 aa, Artificial sequence
  Synthetic antibody
DVVMTQTPSSVSAAVGGTVTIKCQASQSIASYLAWYQQKPGQPPKLLIYRASTLASGVPSRFKGSGSGTEYTLTISDLECADAATYYCQTTYVGGSGNAFGGGTKVEIK

SEQ ID NO: 123 - protein, 111 aa, Artificial sequence
  Synthetic antibody
DPMLTQTPSPVSAAVGGTVTITCQASQSVYNNNYLSWFQQKPGQPPKLLIYKASILASGVPSRFKGSGSGTQFTLTISDLQCDDAATYYCLGGYSSPSDDAFGGGTKVVVE

SEQ ID NO: 124 - protein, 112 aa, Artificial sequence
  Synthetic antibody
DVVMTQTPSSVEAGVGGTVTIKCQASESSSRHLSWYQQKPGQRPKLLIYWASTLASGVPSRFQGSGSGTEFTLTISDLECADAATYYCQSYYGLSNNGWRYAFGGGTEVVVK

SEQ ID NO: 125 - protein, 110 aa, Artificial sequence
  Synthetic antibody
AIKMTQTPSSVSAAVGGTVTINCQASEDIENYLAWYQQKPGQPPKLLIYRASTLASGVSSRFKGSGSGTEFTLTISDLECADAATYWCQQGYSGSNVDNFFGGGTKVVVE

SEQ ID NO: 126 - protein, 110 aa, Artificial sequence
  Synthetic antibody
ALVMTQTPASVEAAVGGTVTIKCQASQSIGSNLAWYQQKPGQPPKLLIYSASTLASGVSSRFKGSRSGTEFTLTISDLECADAATYYCQFTYYGSSDVDVFGGGTKVVVE

SEQ ID NO: 127 - protein, 112 aa, Artificial sequence
  Synthetic antibody
DPVLTQTPASVEAAVGGTVTIKCQASQSIGSILAWYQQKPGQPPHLLIYAAAKLASGVPSRFKGSGSGTQFTLTISGVECDDAATYYCQSYYYSVTTSYSNSFGGGTEVVVK

SEQ ID NO: 128 - protein, 110 aa, Artificial sequence
  Synthetic antibody
DPVLTQTPASVEVPVGGTVTINCQASQSIGKYLNWYQQKPGQPPKLLIYSSSSLASGVSSRFKGSGFGTQFTLTISGVQCADAATYYCQQGYSYVDLENGFGGGTELEIL

SEQ ID NO: 129 - protein, 112 aa, Artificial sequence
  Synthetic antibody
DVVMTQTPASVSEPVGGTVTIKCQASQSISSYLTWYQQKPGQPPKLLIYKASTLASGVSSRFKGSGSGTEFTLTISDLECADAATYYCQSSYYGPSDVDFSPFGGGTEVEIK

SEQ ID NO: 130 - protein, 110 aa, Artificial sequence
  Synthetic antibody
ALVMTQTPASVEAAVGGTVTIRCQASERIGNALAWYQQKPGQPPKLLIYQTSKLTSGVPSRFKGSGSGTEHTLTISDLECADAATYYCQSYYYTGSISYEFGGGTKVVVE

SEQ ID NO: 131 - protein, 112 aa, Artificial sequence
  Synthetic antibody
AQVLTQTPASVEAAVGGTVTIKCQASQSISSYLAWYQQKPGQPPKLLIYRASTLASGVPSRFKGSGSGTEFTLTISDLECADAATYYCQSDYYSSSSNNGFVFGGGTEVVVK

SEQ ID NO: 132 - protein, 111 aa, Artificial sequence
  Synthetic antibody
DVVMTQTPASVSAAVGGTVTIKCQASEDIYSLLAWYQQKPGQPPKFLIYDASDLASGVPSRFSGSGSGTEYTLTINDLKCDDAATYYCQGGYYSGDGTDIPFGGGTELEIL

SEQ ID NO: 133 - protein, 125 aa, Artificial sequence
  Synthetic antibody
EVQLVESGGGLVQPGGSLRLSCAASGFTISSDYYMCWVRQAPGKGLEWIGCIYIGSGTDTYYADSVKGRFTISKHNSKNTLYLQMTSLRAEDTAVYYCARDKGWSNAWGFYFQLWGQGTLVTVSS

SEQ ID NO: 134 - protein, 110 aa, Artificial sequence
  Synthetic antibody
DIQMTQSPSSLSASVGDRVTITCQASQSIGKYLNWYQQKPGKAPKLLIYSSSSLASGVPSRFSGSGFGTDFTLTISSLQPEDFATYYCQQGYSYVDLENGFGGGTKVEIK

SEQ ID NO: 135 - protein, 118 aa, Artificial sequence
  Synthetic antibody
QVQLVESGGGVVQPGRSLRLSCAASGFTLTTYWVCWVRQAPGKGLEWVACIDAGSITYYTTWARGRFTISRDNSTNTLFLQMNSLRAEDTAVYYCARDAHYTYALVLWGRGTLVTVSS

SEQ ID NO: 136 - protein, 109 aa, Artificial sequence
  Synthetic antibody
DIQMTQSPSSVSASVGDRVTIKCQASEDIESYLAWYQQKPGKAPKLLIYDASDLASGVPSRFSGSGSGTDFTLTISSLQPEDAATYYCQGYSYGSSYVGFGGGTKVEIK

SEQ ID NO: 137 - DNA, 375 bp, Artificial sequence
  Synthetic antibody
gaggtgcagctcgtagaaagtgggggcggccttgtgcagccgggtggtagtcttcgcctgtcctgtgcggcgagcggatttacaatatcatccgactactatatgtgctgggtccgccaggcgcccggaaaaggtttggaatggatcggctgcatttacataggatctggcacagacacttactatgcggacagcgtgaaaggaagattcactattagcaagcacaacagcaaaaatactttgtatctgcagatgaccagcctcagggccgaggataccgcggtctactactgcgctagggataagggttggtcaaacgcatgggggttctacttccagttgtggggacagggaactcttgtcaccgtgagttcc

SEQ ID NO: 138 - DNA, 330 bp, Artificial sequence
  Synthetic antibody
gacatccagatgacccagtctccgtcaagtctcagtgcctccgtgggagaccgggtgactataacatgccaagcgtcacaaagtatcgggaagtacctcaactggtatcaacaaaagcctggcaaggcaccaaagctcctcatctattccagtagttctctcgcaagtggcgttccgtccaggttttctggctctggattcggtaccgatttcacgctcactatttcaagtttgcagcctgaggattttgccacttactactgtcaacaggggtacagttatgtggacttggagaacggctttggaggcggaacgaaggttgaaataaag

SEQ ID NO: 139 - DNA, 354 bp, Artificial sequence
  Synthetic antibody
caagttcagttggtagaatctggcggaggtgtggtgcagcctgggaggtctctgagattgtcctgcgctgcgagcgggtttaccctcacgacgtattgggtttgctgggtccggcaggcacctggtaaagggttggagtgggtcgcttgcatcgacgctggctccattacatactataccacgtgggcgcggggtagatttactatttcacgagacaactctaccaacactttgttcttgcagatgaattccttgcgagcagaggacactgcagtatattattgtgcgagagacgcacattatacttacgcactggtgctgtggggtagaggtactctcgtgacggtttcttct

SEQ ID NO: 140 - DNA, 327 bp, Artificial sequence
  Synthetic antibody
gatatacaaatgacacaaagcccctccagcgtatccgcctctgtaggggatcgggtgacaatcaagtgtcaagcatccgaagatattgaaagctacctcgcatggtatcaacaaaagcctggaaaggcgccgaaattgcttatttatgatgcttccgacctggcttctggcgttccaagccgattctctggaagcgggagtggaacagactttacgctgactataagctctcttcaaccggaggacgccgcgacatattattgccaggggtattcctatggctctagctacgttggtttcgggggaggcactaaagtcgagattaag

SEQ ID NO: 141 - protein, 8 aa, Artificial sequence
  Synthetic peptide
  misc_feature (2)..(3): Xaa can be any naturally occurring amino acid
  misc_feature (5)..(5): Xaa can be any naturally occurring amino acid
  misc_feature (7)..(7): Xaa can be any naturally occurring amino acid
DXXDXDXE

SEQ ID NO: 142 - protein, 8 aa, Artificial sequence
  Synthetic peptide
DGLDLAWL

SEQ ID NO: 143 - DNA, 20 bp, Artificial sequence
  Synthetic primer
accgtgccagtgttgttctg

SEQ ID NO: 144 - DNA, 23 bp, Artificial sequence
  Synthetic primer
agcacctgtggttgttattttgt

SEQ ID NO: 145 - DNA, 23 bp, Artificial sequence
  Synthetic primer
agcacctgtggttgttattttgt

SEQ ID NO: 146 - DNA, 21 bp, Artificial sequence
  Synthetic primer
agtagttccgcactgggtaca

SEQ ID NO: 147 - DNA, 22 bp, Artificial sequence
  Synthetic primer
tgtcatctgtgaaaaggtggtc

SEQ ID NO: 148 - DNA, 20 bp, Artificial sequence
  Synthetic primer
actggagcagcggtgttatg

SEQ ID NO: 149 - DNA, 19 bp, Artificial sequence
  Synthetic primer
gtggcgttgacatcgttgg

SEQ ID NO: 150 - DNA, 23 bp, Artificial sequence
  Synthetic primer
ctatgtacttcgtatgcaggtcg

SEQ ID NO: 151 - DNA, 22 bp, Artificial sequence
  Synthetic primer
gtgatgctcaggtatccatcca

SEQ ID NO: 152 - DNA, 21 bp, Artificial sequence
  Synthetic primer
cacagttctcaaagcacagcg

SEQ ID NO: 153 - DNA, 19 bp, Artificial sequence
  Synthetic primer
agggcctccatctggactg

SEQ ID NO: 154 - DNA, 23 bp, Artificial sequence
  Synthetic primer
gtggttctggtaatcatgctctg

SEQ ID NO: 155 - DNA, 21 bp, Artificial sequence
  Synthetic primer
ccagacttttgctactgggac

SEQ ID NO: 156 - DNA, 21 bp, Artificial sequence
  Synthetic primer
gcttgttcggcagtgatagag

SEQ ID NO: 157 - DNA, 21 bp, Artificial sequence
  Synthetic primer
ctctttggggacgacttcatc

SEQ ID NO: 158 - DNA, 21 bp, Artificial sequence
  Synthetic primer
tcccgagaatggagaaggaac

SEQ ID NO: 159 - protein, 8 aa, Artificial sequence
  Synthetic antibody
GWDLSDTY

SEQ ID NO: 160 - protein, 9 aa, Artificial sequence
  Synthetic antibody
GFSFSSGYY

SEQ ID NO: 161 - protein, 8 aa, Artificial sequence
  Synthetic antibody
EFDFSSYY

SEQ ID NO: 162 - protein, 9 aa, Artificial sequence
  Synthetic antibody
GLDFSSSYW

SEQ ID NO: 163 - protein, 8 aa, Artificial sequence
  Synthetic antibody
GFTLTTYW

SEQ ID NO: 164 - protein, 9 aa, Artificial sequence
  Synthetic antibody
GFSFSNNNY

SEQ ID NO: 165 - protein, 9 aa, Artificial sequence
  Synthetic antibody
GFSFSSSYW

SEQ ID NO: 166 - protein, 9 aa, Artificial sequence
  Synthetic antibody
GFSFSGAYW

SEQ ID NO: 167 - protein, 8 aa, Artificial sequence
  Synthetic antibody
GFDFSSNA

SEQ ID NO: 168 - protein, 8 aa, Artificial sequence
  Synthetic antibody
GFDFSSNA

SEQ ID NO: 169 - protein, 8 aa, Artificial sequence
  Synthetic antibody
GLTLSRYW

SEQ ID NO: 170 - protein, 9 aa, Artificial sequence
  Synthetic antibody
GFSFSSSYW

SEQ ID NO: 171 - protein, 8 aa, Artificial sequence
  Synthetic antibody
GFDFSRNA

SEQ ID NO: 172 - protein, 9 aa, Artificial sequence
  Synthetic antibody
GFTISSTFY

SEQ ID NO: 173 - protein, 9 aa, Artificial sequence
  Synthetic antibody
GFSFSSSHY

SEQ ID NO: 174 - protein, 9 aa, Artificial sequence
  Synthetic antibody
GFSFSSSYY

SEQ ID NO: 175 - protein, 9 aa, Artificial sequence
  Synthetic antibody
GFSFSSGYY

SEQ ID NO: 176 - protein, 8 aa, Artificial sequence
  Synthetic antibody
GFDFSSYY

SEQ ID NO: 177 - protein, 8 aa, Artificial sequence
  Synthetic antibody
GIDFSSNV

SEQ ID NO: 178 - protein, 9 aa, Artificial sequence
  Synthetic antibody
GFSFSSSYY

SEQ ID NO: 179 - protein, 9 aa, Artificial sequence
  Synthetic antibody
GFSFSYRYY

SEQ ID NO: 180 - protein, 9 aa, Artificial sequence
  Synthetic antibody
GFSFSSGYY

SEQ ID NO: 181 - protein, 9 aa, Artificial sequence
  Synthetic antibody
GFSFSDNYW

SEQ ID NO: 182 - protein, 9 aa, Artificial sequence
  Synthetic antibody
GFDFSSYYY

SEQ ID NO: 183 - protein, 9 aa, Artificial sequence
  Synthetic antibody
GFSFSSSHY

SEQ ID NO: 184 - protein, 9 aa, Artificial sequence
  Synthetic antibody
GFSFSSDYD

SEQ ID NO: 185 - protein, 9 aa, Artificial sequence
  Synthetic antibody
GFSFSSSYW

SEQ ID NO: 186 - protein, 9 aa, Artificial sequence
  Synthetic antibody
GFSFSSGYW

SEQ ID NO: 187 - protein, 9 aa, Artificial sequence
  Synthetic antibody
GFTISSDYY

SEQ ID NO: 188 - protein, 9 aa, Artificial sequence
  Synthetic antibody
GFSFSSSYW

SEQ ID NO: 189 - protein, 9 aa, Artificial sequence
  Synthetic antibody
GFSFSNNYW

SEQ ID NO: 190 - protein, 9 aa, Artificial sequence
  Synthetic antibody
GFSFSSSYY

SEQ ID NO: 191 - protein, 9 aa, Artificial sequence
  Synthetic antibody
GFSFSGPYW

SEQ ID NO: 192 - protein, 7 aa, Artificial sequence
  Synthetic antibody
CIYNGNG

SEQ ID NO: 193 - protein, 7 aa, Artificial sequence
  Synthetic antibody
IGASSGL

SEQ ID NO: 194 - protein, 8 aa, Artificial sequence
  Synthetic antibody
IYAGKGDT

SEQ ID NO: 195 - protein, 7 aa, Artificial sequence
  Synthetic antibody
INTSSGS

SEQ ID NO: 196 - protein, 7 aa, Artificial sequence
  Synthetic antibody
IDAGSIT

SEQ ID NO: 197 - protein, 8 aa, Artificial sequence
  Synthetic antibody
IYTGDAYT

SEQ ID NO: 198 - protein, 8 aa, Artificial sequence
  Synthetic antibody
IYTGDGAN

SEQ ID NO: 199 - protein, 9 aa, Artificial sequence
  Synthetic antibody
IYVISSSNT

SEQ ID NO: 200 - protein, 8 aa, Artificial sequence
  Synthetic antibody
FDYISDSI

SEQ ID NO: 201 - protein, 8 aa, Artificial sequence
  Synthetic antibody
IYNGDAST

SEQ ID NO: 202 - protein, 7 aa, Artificial sequence
  Synthetic antibody
IDAGSIT

SEQ ID NO: 203 - protein, 9 aa, Artificial sequence
  Synthetic antibody
IDAGSSEST

SEQ ID NO: 204 - protein, 9 aa, Artificial sequence
  Synthetic antibody
IDAGSSEST

SEQ ID NO: 205 - protein, 9 aa, Artificial sequence
  Synthetic antibody
MHVGSATTT

SEQ ID NO: 206 - protein, 8 aa, Artificial sequence
  Synthetic antibody
IHISSGVT

SEQ ID NO: 207 - protein, 8 aa, Artificial sequence
  Synthetic antibody
IYNGDGST

SEQ ID NO: 208 - protein, 9 aa, Artificial sequence
  Synthetic antibody
IYGAVSDRI

SEQ ID NO: 209 - protein, 8 aa, Artificial sequence
  Synthetic antibody
IYGGKGTT

SEQ ID NO: 210 - protein, 9 aa, Artificial sequence
  Synthetic antibody
IDTDDFIGT

SEQ ID NO: 211 - protein, 9 aa, Artificial sequence
  Synthetic antibody
IYAGSSGST

SEQ ID NO: 212 - protein, 8 aa, Artificial sequence
  Synthetic antibody
IDNGDGDT

SEQ ID NO: 213 - protein, 8 aa, Artificial sequence
  Synthetic antibody
IGASSGLT

SEQ ID NO: 214 - protein, 9 aa, Artificial sequence
  Synthetic antibody
IYIGFSGTT

SEQ ID NO: 215 - protein, 8 aa, Artificial sequence
  Synthetic antibody
IDTGSGET

SEQ ID NO: 216 - protein, 8 aa, Artificial sequence
  Synthetic antibody
IYLGDDST

SEQ ID NO: 217 - protein, 9 aa, Artificial sequence
  Synthetic antibody
IDGGSSGEI

SEQ ID NO: 218 - protein, 9 aa, Artificial sequence
  Synthetic antibody
IYPSSSGST

SEQ ID NO: 219 - protein, 8 aa, Artificial sequence
  Synthetic antibody
IGASYGGT

SEQ ID NO: 220 - protein, 9 aa, Artificial sequence
  Synthetic antibody
IYIGSGTDT

SEQ ID NO: 221 - protein, 9 aa, Artificial sequence
  Synthetic antibody
IYAGGSGST

SEQ ID NO: 222 - protein, 9 aa, Artificial sequence
  Synthetic antibody
IYAGDTGTA

SEQ ID NO: 223 - protein, 8 aa, Artificial sequence
  Synthetic antibody
IFTGGVST

SEQ ID NO: 224 - protein, 9 aa, Artificial sequence
  Synthetic antibody
IGARSSGST

SEQ ID NO: 225 - protein, 8 aa, Artificial sequence
  Synthetic antibody
ARGVAADL

SEQ ID NO: 226 - protein, 15 aa, Artificial sequence
  Synthetic antibody
ARVYLGGADYERFNL

SEQ ID NO: 227 - protein, 13 aa, Artificial sequence
  Synthetic antibody
ARGWLGSDYYFNF

SEQ ID NO: 228 - protein, 10 aa, Artificial sequence
  Synthetic antibody
AKYINSYFGL

SEQ ID NO: 229 - protein, 12 aa, Artificial sequence
  Synthetic antibody
ARDAHYTYALVL

SEQ ID NO: 230 - protein, 15 aa, Artificial sequence
  Synthetic antibody
ARGYSGYSGYGYSDL

SEQ ID NO: 231 - protein, 13 aa, Artificial sequence
  Synthetic antibody
ARSAYGGYGYFNF

SEQ ID NO: 232 - protein, 14 aa, Artificial sequence
  Synthetic antibody
ARYSCSSAFCLFNL

SEQ ID NO: 233 - protein, 14 aa, Artificial sequence
  Synthetic antibody
ARDNVGDGYTTFDL

SEQ ID NO: 234 - protein, 12 aa, Artificial sequence
  Synthetic antibody
ARCFNRFYRGDL

SEQ ID NO: 235 - protein, 12 aa, Artificial sequence
  Synthetic antibody
ARDADYTYALVL

SEQ ID NO: 236 - protein, 16 aa, Artificial sequence
  Synthetic antibody
ARDSRVVAGVGMYLNL

SEQ ID NO: 237 - protein, 13 aa, Artificial sequence
  Synthetic antibody
AGSPYSGYGYFNL

SEQ ID NO: 238 - protein, 12 aa, Artificial sequence
  Synthetic antibody
ARYVNSGYYMDS

SEQ ID NO: 239 - protein, 16 aa, Artificial sequence
  Synthetic antibody
ARDDRYGHGGYPYTDL

SEQ ID NO: 240 - protein, 13 aa, Artificial sequence
  Synthetic antibody
ARDADYAGVVLNL

SEQ ID NO: 241 - protein, 18 aa, Artificial sequence
  Synthetic antibody
ARDSYSGSTYYRSYYFNL

SEQ ID NO: 242 - protein, 14 aa, Artificial sequence
  Synthetic antibody
ARDYVSSSGYYMDL

SEQ ID NO: 243 - protein, 10 aa, Artificial sequence
  Synthetic antibody
ARGDAGYYAL

SEQ ID NO: 244 - protein, 18 aa, Artificial sequence
  Synthetic antibody
ARDRYGGGSYYIPYYFDL

SEQ ID NO: 245 - protein, 12 aa, Artificial sequence
  Synthetic antibody
ARGSSGYNGFNF

SEQ ID NO: 246 - protein, 15 aa, Artificial sequence
  Synthetic antibody
ARVYLGGADYEQFNL

SEQ ID NO: 247 - protein, 17 aa, Artificial sequence
  Synthetic antibody
ARGVFYVGSGYWYYLNL

SEQ ID NO: 248 - protein, 10 aa, Artificial sequence
  Synthetic antibody
ARDPYFGTNL

SEQ ID NO: 249 - protein, 14 aa, Artificial sequence
  Synthetic antibody
ARHGYNLAIHNYII

SEQ ID NO: 250 - protein, 12 aa, Artificial sequence
  Synthetic antibody
ARCIWSHNYYDL

SEQ ID NO: 251 - protein, 17 aa, Artificial sequence
  Synthetic antibody
ARDIPYNIHGYTTGFNL

SEQ ID NO: 252 - protein, 12 aa, Artificial sequence
  Synthetic antibody
TRDDGGLDNPNL

SEQ ID NO: 253 - protein, 16 aa, Artificial sequence
  Synthetic antibody
ARDKGWSNAWGFYFQL

SEQ ID NO: 254 - protein, 14 aa, Artificial sequence
  Synthetic antibody
ARDGVSGSGWFFNL

SEQ ID NO: 255 - protein, 15 aa, Artificial sequence
  Synthetic antibody
ARDGDGVGNGGTFAL

SEQ ID NO: 256 - protein, 16 aa, Artificial sequence
  Synthetic antibody
ARTYTAGFTGDNYFNL

SEQ ID NO: 257 - protein, 19 aa, Artificial sequence
  Synthetic antibody
ARSYPAYAGDGYALYYFNL

SEQ ID NO: 258 - protein, 6 aa, Artificial sequence
  Synthetic antibody
ESIDND

SEQ ID NO: 259 - protein, 6 aa, Artificial sequence
  Synthetic antibody
ESISSR

SEQ ID NO: 260 - protein, 6 aa, Artificial sequence
  Synthetic antibody
EDIESY

SEQ ID NO: 261 - protein, 6 aa, Artificial sequence
  Synthetic antibody
QNIYSN

SEQ ID NO: 262 - protein, 6 aa, Artificial sequence
  Synthetic antibody
EDIESY

SEQ ID NO: 263 - protein, 6 aa, Artificial sequence
  Synthetic antibody
QSIYSY

SEQ ID NO: 264 - protein, 6 aa, Artificial sequence
  Synthetic antibody
EDIYSL

SEQ ID NO: 265 - protein, 6 aa, Artificial sequence
  Synthetic antibody
QSVSNL

SEQ ID NO: 266 - protein, 6 aa, Artificial sequence
  Synthetic antibody
QSISSW

SEQ ID NO: 267 - protein, 8 aa, Artificial sequence
  Synthetic antibody
QSVYDNDW

SEQ ID NO: 268 - protein, 6 aa, Artificial sequence
  Synthetic antibody
EDIESY

SEQ ID NO: 269 - protein, 6 aa, Artificial sequence
  Synthetic antibody
QSIGIY

SEQ ID NO: 270 - protein, 6 aa, Artificial sequence
  Synthetic antibody
QSISTA

SEQ ID NO: 271 - protein, 6 aa, Artificial sequence
  Synthetic antibody
QTISNL

SEQ ID NO: 272 - protein, 6 aa, Artificial sequence
  Synthetic antibody
ESISSW

SEQ ID NO: 273 - protein, 6 aa, Artificial sequence
  Synthetic antibody
ESIGNA

SEQ ID NO: 274 - protein, 8 aa, Artificial sequence
  Synthetic antibody
ENIYVDKY

SEQ ID NO: 275 - protein, 6 aa, Artificial sequence
  Synthetic antibody
EDIYSL

SEQ ID NO: 276 - protein, 6 aa, Artificial sequence
  Synthetic antibody
QSIGSY

SEQ ID NO: 277 - protein, 8 aa, Artificial sequence
  Synthetic antibody
QNIYVDSY

SEQ ID NO: 278 - protein, 6 aa, Artificial sequence
  Synthetic antibody
QSIGSY

SEQ ID NO: 279 - protein, 6 aa, Artificial sequence
  Synthetic antibody
ESVSSR

SEQ ID NO: 280 - protein, 6 aa, Artificial sequence
  Synthetic antibody
QSIASY

SEQ ID NO: 281 - protein, 8 aa, Artificial sequence
  Synthetic antibody
QSVYNNNY

SEQ ID NO: 282 - protein, 6 aa, Artificial sequence
  Synthetic antibody
ESSSRH

SEQ ID NO: 283 - protein, 6 aa, Artificial sequence
  Synthetic antibody
EDIENY

SEQ ID NO: 284 - protein, 6 aa, Artificial sequence
  Synthetic antibody
QSIGSN

SEQ ID NO: 285 - protein, 6 aa, Artificial sequence
  Synthetic antibody
QSIGSI

SEQ ID NO: 286 - protein, 6 aa, Artificial sequence
  Synthetic antibody
QSIGKY

SEQ ID NO: 287 - protein, 6 aa, Artificial sequence
  Synthetic antibody
QSISSY

SEQ ID NO: 288 - protein, 6 aa, Artificial sequence
  Synthetic antibody
ERIGNA

SEQ ID NO: 289 - protein, 6 aa, Artificial sequence
  Synthetic antibody
QSISSY

SEQ ID NO: 290 - protein, 6 aa, Artificial sequence
  Synthetic antibody
EDIYSL

SEQ ID NO: 291 - protein, 13 aa, Artificial sequence
  Synthetic antibody
QSNYYSGSSSSYG

SEQ ID NO: 292 - protein, 13 aa, Artificial sequence
  Synthetic antibody
QSYYYSGGVPSYG

SEQ ID NO: 293 - protein, 13 aa, Artificial sequence
  Synthetic antibody
QGAWYSSSVDMYA

SEQ ID NO: 294 - protein, 12 aa, Artificial sequence
  Synthetic antibody
QQDYSWSNVDNV

SEQ ID NO: 295 - protein, 11 aa, Artificial sequence
  Synthetic antibody
QGYSYGSSYVG

SEQ ID NO: 296 - protein, 12 aa, Artificial sequence
  Synthetic antibody
QQGYTSYNIDNA

SEQ ID NO: 297 - protein, 15 aa, Artificial sequence
  Synthetic antibody
QNYHYSSTTSYAPNP

SEQ ID NO: 298 - protein, 5 aa, Artificial sequence
  Synthetic antibody
QSGYY

SEQ ID NO: 299 - protein, 14 aa, Artificial sequence
  Synthetic antibody
QSNYGSYSSSYGYA

SEQ ID NO: 300 - protein, 11 aa, Artificial sequence
  Synthetic antibody
AGDYINTIDNA

SEQ ID NO: 301 - protein, 11 aa, Artificial sequence
  Synthetic antibody
QGYYYGAIYVA

SEQ ID NO: 302 - protein, 12 aa, Artificial sequence
  Synthetic antibody
QQGDTIYNVDNT

SEQ ID NO: 303 - protein, 11 aa, Artificial sequence
  Synthetic antibody
QSYYDSSAGNT

SEQ ID NO: 304 - protein, 12 aa, Artificial sequence
  Synthetic antibody
QQGWSSSNVDNV

SEQ ID NO: 305 - protein, 12 aa, Artificial sequence
  Synthetic antibody
QSTYYNENVGNA

SEQ ID NO: 306 - protein, 11 aa, Artificial sequence
  Synthetic antibody
QCTWYGNVYLA

SEQ ID NO: 307 - protein, 11 aa, Artificial sequence
  Synthetic antibody
AGSFSSSDSCA

SEQ ID NO: 308 - protein, 13 aa, Artificial sequence
  Synthetic antibody
QGGYYSGSPTDNS

SEQ ID NO: 309 - protein, 13 aa, Artificial sequence
  Synthetic antibody
QCSYFGSGAVDSP

SEQ ID NO: 310 - protein, 11 aa, Artificial sequence
  Synthetic antibody
AGGYSADSDNA

SEQ ID NO: 311 - protein, 12 aa, Artificial sequence
  Synthetic antibody
QCGYYDSNYGGA

SEQ ID NO: 312 - protein, 10 aa, Artificial sequence
  Synthetic antibody
QNYYGISGVA

SEQ ID NO: 313 - protein, 11 aa, Artificial sequence
  Synthetic antibody
QTTYVGGSGNA

SEQ ID NO: 314 - protein, 11 aa, Artificial sequence
  Synthetic antibody
LGGYSSPSDDA

SEQ ID NO: 315 - protein, 14 aa, Artificial sequence
  Synthetic antibody
QSYYGLSNNGWRYA

SEQ ID NO: 316 - protein, 12 aa, Artificial sequence
  Synthetic antibody
QQGYSGSNVDNF

SEQ ID NO: 317 - protein, 12 aa, Artificial sequence
  Synthetic antibody
QFTYYGSSDVDV

SEQ ID NO: 318 - protein, 14 aa, Artificial sequence
  Synthetic antibody
QSYYYSVTTSYSNS

SEQ ID NO: 319 - protein, 12 aa, Artificial sequence
  Synthetic antibody
QQGYSYVDLENG

SEQ ID NO: 320 - protein, 14 aa, Artificial sequence
  Synthetic antibody
QSSYYGPSDVDFSP

SEQ ID NO: 321 - protein, 12 aa, Artificial sequence
  Synthetic antibody
QSYYYTGSISYE

SEQ ID NO: 322 - protein, 14 aa, Artificial sequence
  Synthetic antibody
QSDYYSSSSNNGFV

SEQ ID NO: 323 - protein, 13 aa, Artificial sequence
  Synthetic antibody
QGGYYSGDGTDIP

SEQ ID NO: 324 - DNA, 27 bp, Artificial sequence
  Synthetic antibody
gggtgggacttaagtgatacctactgg

SEQ ID NO: 325 - DNA, 24 bp, Artificial sequence
  Synthetic antibody
atttataatggtaatggtgacact

SEQ ID NO: 326 - DNA, 24 bp, Artificial sequence
  Synthetic antibody
gcgagaggagtggctgcggacttg

SEQ ID NO: 327 - DNA, 18 bp, Artificial sequence
  Synthetic antibody
gagagcattgataatgat

SEQ ID NO: 328 - DNA, 39 bp, Artificial sequence
  Synthetic antibody
caaagtaattattatagtggtagtagtagtagttatggt

SEQ ID NO: 329 - DNA, 27 bp, Artificial sequence
  Synthetic antibody
ggattctccttcagtagcggctattac

SEQ ID NO: 330 - DNA, 24 bp, Artificial sequence
  Synthetic antibody
attggtgctagtagtggactcact

SEQ ID NO: 331 - DNA, 45 bp, Artificial sequence
  Synthetic antibody
gcgagagtctatcttggtggtgctgattatgagcgatttaatttg

SEQ ID NO: 332 - DNA, 18 bp, Artificial sequence
  Synthetic antibody
gagagcattagtagtaga

SEQ ID NO: 333 - DNA, 39 bp, Artificial sequence
  Synthetic antibody
caaagttattattatagtggtggggttcctagttatggt

SEQ ID NO: 334 - DNA, 24 bp, Artificial sequence
  Synthetic antibody
gaattcgacttcagtagctactac

SEQ ID NO: 335 - DNA, 24 bp, Artificial sequence
  Synthetic antibody
atttatgctggtaaaggtgacaca

SEQ ID NO: 336 - DNA, 39 bp, Artificial sequence
  Synthetic antibody
gcgagagggtggctggggtccgattactactttaacttt

SEQ ID NO: 337 - DNA, 18 bp, Artificial sequence
  Synthetic antibody
gaggacattgaaagctat

SEQ ID NO: 338 - DNA, 36 bp, Artificial sequence
  Synthetic antibody
ggtgcttggtatagtagtagtgttgatatgtatgct

SEQ ID NO: 339 - DNA, 27 bp, Artificial sequence
  Synthetic antibody
gggttagacttcagtagcagctactgg

SEQ ID NO: 340 - DNA, 24 bp, Artificial sequence
  Synthetic antibody
attaatactagtagtggtagcaca

SEQ ID NO: 341 - DNA, 30 bp, Artificial sequence
  Synthetic antibody
gcgaaatatattaatagttatttcggcttg

SEQ ID NO: 342 - DNA, 18 bp, Artificial sequence
  Synthetic antibody
cagaacatttacagcaat

SEQ ID NO: 343 - DNA, 36 bp, Artificial sequence
  Synthetic antibody
caacaggattatagttggagtaatgttgataatgtt

SEQ ID NO: 344 - DNA, 24 bp, Artificial sequence
  Synthetic antibody
ggattcaccctcactacctactgg

SEQ ID NO: 345 - DNA, 21 bp, Artificial sequence
  Synthetic antibody
attgatgctggtagtatcact

SEQ ID NO: 346 - DNA, 36 bp, Artificial sequence
  Synthetic antibody
gcgagagatgctcattatacttatgcccttgtcttg

SEQ ID NO: 347 - DNA, 18 bp, Artificial sequence
  Synthetic antibody
gaggacattgaaagttat

SEQ ID NO: 348 - DNA, 33 bp, Artificial sequence
  Synthetic antibody
caaggctattcttatggtagtagttatgttggt

SEQ ID NO: 349 - DNA, 27 bp, Artificial sequence
  Synthetic antibody
ggattctccttcagtaataataactac

SEQ ID NO: 350 - DNA, 24 bp, Artificial sequence
  Synthetic antibody
atttatactggtgatgcttacact

SEQ ID NO: 351 - DNA, 45 bp, Artificial sequence
  Synthetic antibody
gcgagaggttacagtggttatagtggttatggttattccgacttg

SEQ ID NO: 352 - DNA, 18 bp, Artificial sequence
  Synthetic antibody
cagagcatttacagctac

SEQ ID NO: 353 - DNA, 36 bp, Artificial sequence
  Synthetic antibody
caacagggttatactagttataatattgacaatgct

SEQ ID NO: 354 - DNA, 27 bp, Artificial sequence
  Synthetic antibody
ggattctccttcagtagcagctactgg

SEQ ID NO: 355 - DNA, 24 bp, Artificial sequence
  Synthetic antibody
atttatactggtgatggtgctaat

SEQ ID NO: 356 - DNA, 39 bp, Artificial sequence
  Synthetic antibody
gcgagatctgcttatggtggttatggttattttaacttc

SEQ ID NO: 357 - DNA, 18 bp, Artificial sequence
  Synthetic antibody
gaggacatttatagctta

SEQ ID NO: 358 - DNA, 45 bp, Artificial sequence
  Synthetic antibody
caaaactatcattatagtagtactactagttatgcccccaatcct

SEQ ID NO: 359 - DNA, 27 bp, Artificial sequence
  Synthetic antibody
ggattctccttcagtggtgcctactgg

SEQ ID NO: 360 - DNA, 27 bp, Artificial sequence
  Synthetic antibody
atttatgtgattagtagtagtaacact

SEQ ID NO: 361 - DNA, 42 bp, Artificial sequence
  Synthetic antibody
gcgagatattcttgtagtagtgctttttgcctttttaacttg

SEQ ID NO: 362 - DNA, 18 bp, Artificial sequence
  Synthetic antibody
cagagtgttagcaacctc

SEQ ID NO: 363 - DNA, 15 bp, Artificial sequence
  Synthetic antibody
caaagtggttattac

SEQ ID NO: 364 - DNA, 24 bp, Artificial sequence
  Synthetic antibody
ggattcgacttcagtagcaatgca

SEQ ID NO: 365 - DNA, 24 bp, Artificial sequence
  Synthetic antibody
tttgattatattagtgatagtata

SEQ ID NO: 366 - DNA, 42 bp, Artificial sequence
  Synthetic antibody
gcgagagacaatgttggtgatggttatactacatttgacttg

SEQ ID NO: 367 - DNA, 18 bp, Artificial sequence
  Synthetic antibody
cagagcattagcagttgg

SEQ ID NO: 368 - DNA, 42 bp, Artificial sequence
  Synthetic antibody
caaagcaattatggtagttatagtagtagttatggatatgct

SEQ ID NO: 369 - DNA, 24 bp, Artificial sequence
  Synthetic antibody
ggattcgacttcagtagcaatgca

SEQ ID NO: 370 - DNA, 24 bp, Artificial sequence
  Synthetic antibody
atttataatggtgatgccagtaca

SEQ ID NO: 371 - DNA, 36 bp, Artificial sequence
  Synthetic antibody
gcgagatgttttaatagattttacagaggagacttg

SEQ ID NO: 372 - DNA, 24 bp, Artificial sequence
  Synthetic antibody
cagagtgtttatgataacgactgg

SEQ ID NO: 373 - DNA, 33 bp, Artificial sequence
  Synthetic antibody
gcaggcgactatattaatactattgataatgct

SEQ ID NO: 374 - DNA, 24 bp, Artificial sequence
  Synthetic antibody
ggactcaccctcagtagatactgg

SEQ ID NO: 375 - DNA, 21 bp, Artificial sequence
  Synthetic antibody
attgatgctggtagtatcact

SEQ ID NO: 376 - DNA, 36 bp, Artificial sequence
  Synthetic antibody
gcgagagatgctgattatacttatgcacttgtcttg

SEQ ID NO: 377 - DNA, 18 bp, Artificial sequence
  Synthetic antibody
gaggacattgaaagctat

SEQ ID NO: 378 - DNA, 33 bp, Artificial sequence
  Synthetic antibody
caaggctattattatggtgctatttatgttgct

SEQ ID NO: 379 - DNA, 27 bp, Artificial sequence
  Synthetic antibody
ggattctccttcagtagcagctactgg

SEQ ID NO: 380 - DNA, 27 bp, Artificial sequence
  Synthetic antibody
attgatgctggtagtagtgagagcact

SEQ ID NO: 381 - DNA, 48 bp, Artificial sequence
  Synthetic antibody
gcgagagattctagggtagtggctggggtggggatgtacttgaatttg

SEQ ID NO: 382 - DNA, 18 bp, Artificial sequence
  Synthetic antibody
cagagcattggtatctac

SEQ ID NO: 383 - DNA, 36 bp, Artificial sequence
  Synthetic antibody
caacagggtgatactatttataatgttgataatact

SEQ ID NO: 384 - DNA, 24 bp, Artificial sequence
  Synthetic antibody
ggattcgacttcagtagaaatgca

SEQ ID NO: 385 - DNA, 27 bp, Artificial sequence
  Synthetic antibody
atttatgttggtagtggtggtggcact

SEQ ID NO: 386 - DNA, 39 bp, Artificial sequence
  Synthetic antibody
gcgggatccccctattctggttatggctactttaacttg

SEQ ID NO: 387 - DNA, 18 bp, Artificial sequence
  Synthetic antibody
cagagcattagcactgca

SEQ ID NO: 388 - DNA, 33 bp, Artificial sequence
  Synthetic antibody
caaagctattatgatagtagtgctggtaatact

SEQ ID NO: 389 - DNA, 27 bp, Artificial sequence
  Synthetic antibody
ggattcaccatcagtagtactttctac

SEQ ID NO: 390 - DNA, 27 bp, Artificial sequence
  Synthetic antibody
atgcatgttggtagtgctactaccact

SEQ ID NO: 391 - DNA, 36 bp, Artificial sequence
  Synthetic antibody
gcgagatatgttaatagtggttattatatggactcg

SEQ ID NO: 392 - DNA, 18 bp, Artificial sequence
  Synthetic antibody
cagaccattagcaacctc

SEQ ID NO: 393 - DNA, 36 bp, Artificial sequence
  Synthetic antibody
caacagggttggagtagtagtaatgttgataatgtt

SEQ ID NO: 394 - DNA, 27 bp, Artificial sequence
  Synthetic antibody
ggattctccttcagtagcagccactat

SEQ ID NO: 395 - DNA, 24 bp, Artificial sequence
  Synthetic antibody
atccatattagtagtggtgtcaca

SEQ ID NO: 396 - DNA, 48 bp, Artificial sequence
  Synthetic antibody
gcgagagacgatcgttatggtcatggtggttatccttatactgacttg

SEQ ID NO: 397 - DNA, 18 bp, Artificial sequence
  Synthetic antibody
gagagcattagcagttgg

SEQ ID NO: 398 - DNA, 36 bp, Artificial sequence
  Synthetic antibody
caatctacttattataatgagaatgttggaaatgcg

SEQ ID NO: 399 - DNA, 27 bp, Artificial sequence
  Synthetic antibody
ggattctccttcagtagcagctactac

SEQ ID NO: 400 - DNA, 24 bp, Artificial sequence
  Synthetic antibody
atttataatggtgatggcagcaca

SEQ ID NO: 401 - DNA, 39 bp, Artificial sequence
  Synthetic antibody
gcgagagatgctgattatgctggcgtggtgctcaacttg

SEQ ID NO: 402 - DNA, 18 bp, Artificial sequence
  Synthetic antibody
gagagcattggcaatgca

SEQ ID NO: 403 - DNA, 33 bp, Artificial sequence
  Synthetic antibody
caatgtacttggtatggtaatgtctatcttgct

SEQ ID NO: 404 - DNA, 33 bp, Artificial sequence; identical to SEQ ID NO: 403
  Synthetic antibody

SEQ ID NO: 405 - DNA, 27 bp, Artificial sequence
  Synthetic antibody
atttatggtgctgttagtgataggatt

SEQ ID NO: 406 - DNA, 54 bp, Artificial sequence
  Synthetic antibody
gcgagagatagttatagtggtagtacttattatagatcatactactttaacttg

SEQ ID NO: 407 - DNA, 24 bp, Artificial sequence
  Synthetic antibody
gaaaatatttatgtagacaaatac

SEQ ID NO: 408 - DNA, 33 bp, Artificial sequence
  Synthetic antibody
gcagggagttttagtagtagtgatagttgtgct

SEQ ID NO: 409 - DNA, 24 bp, Artificial sequence
  Synthetic antibody
ggattcgacttcagtagttactac

SEQ ID NO: 410 - DNA, 24 bp, Artificial sequence
  Synthetic antibody
atttatggtggtaaaggtaccaca

SEQ ID NO: 411 - DNA, 42 bp, Artificial sequence
  Synthetic antibody
gcgagagactatgttagtagtagtggttattatatggacttg

SEQ ID NO: 412 - DNA, 18 bp, Artificial sequence
  Synthetic antibody
gaggacatttatagctta

SEQ ID NO: 413 - DNA, 39 bp, Artificial sequence
  Synthetic antibody
caaggcggttattatagtggtagtcctactgataattct

SEQ ID NO: 414 - DNA, 24 bp, Artificial sequence
  Synthetic antibody
ggaatcgacttcagtagcaatgta

SEQ ID NO: 415 - DNA, 27 bp, Artificial sequence
  Synthetic antibody
attgatactgatgattttattggcact

SEQ ID NO: 416 - DNA, 30 bp, Artificial sequence
  Synthetic antibody
gcgaggggagatgctggttactatgccttg

SEQ ID NO: 417 - DNA, 18 bp, Artificial sequence
  Synthetic antibody
cagagcattggtagctat

SEQ ID NO: 418 - DNA, 39 bp, Artificial sequence
  Synthetic antibody
caatgttcttattttggtagtggtgctgtcgatagtcct

SEQ ID NO: 419 - DNA, 27 bp, Artificial sequence
  Synthetic antibody
ggattctccttcagtagcagctactac

SEQ ID NO: 420 - DNA, 27 bp, Artificial sequence
  Synthetic antibody
atttatgctggtagtagtggtagcaca

SEQ ID NO: 421 - DNA, 54 bp, Artificial sequence
  Synthetic antibody
gcgagagaccgttatggtggtggttcttattatataccttactactttgacttg

SEQ ID NO: 422 - DNA, 24 bp, Artificial sequence
  Synthetic antibody
cagaacatttatgtcgacagctac

SEQ ID NO: 423 - DNA, 33 bp, Artificial sequence
  Synthetic antibody
gcaggcggttatagtgctgattcggataatgct

SEQ ID NO: 424 - DNA, 27 bp, Artificial sequence
  Synthetic antibody
ggattctccttcagttacaggtactac

SEQ ID NO: 425 - DNA, 24 bp, Artificial sequence
  Synthetic antibody
attgataatggtgatggcgacaca

SEQ ID NO: 426 - DNA, 36 bp, Artificial sequence
  Synthetic antibody
gcgagagggagtagtggttataatggctttaacttc

SEQ ID NO: 427 - DNA, 18 bp, Artificial sequence
  Synthetic antibody
cagagcattggtagctac

SEQ ID NO: 428 - DNA, 36 bp, Artificial sequence
  Synthetic antibody
caatgtggttattatgatagtaattatgggggtgct

SEQ ID NO: 429 - DNA, 27 bp, Artificial sequence
  Synthetic antibody
ggattctccttcagtagcggctactac

SEQ ID NO: 430 - DNA, 24 bp, Artificial sequence
  Synthetic antibody
attggtgctagtagtggactcact

SEQ ID NO: 431 - DNA, 45 bp, Artificial sequence
  Synthetic antibody
gcgagagtctatcttggtggtgctgattatgagcaatttaatttg

SEQ ID NO: 432 - DNA, 18 bp, Artificial sequence
  Synthetic antibody
gagagcgttagtagtaga

SEQ ID NO: 433 - DNA, 30 bp, Artificial sequence
  Synthetic antibody
caaaactattatggtattagtggtgtcgct

SEQ ID NO: 434 - DNA, 27 bp, Artificial sequence
  Synthetic antibody
ggattctccttcagtgacaactactgg

SEQ ID NO: 435 - DNA, 27 bp, Artificial sequence
  Synthetic antibody
atttatattggttttagtggtaccact

SEQ ID NO: 436 - DNA, 51 bp, Artificial sequence
  Synthetic antibody
gcgagaggggttttttatgttggtagtggttattggtactacttgaacttg

SEQ ID NO: 437 - DNA, 18 bp, Artificial sequence
  Synthetic antibody
cagagcattgctagttac

SEQ ID NO: 438 - DNA, 33 bp, Artificial sequence
  Synthetic antibody
caaactacttatgttggtggtagtggcaatgct

SEQ ID NO: 439 - DNA, 27 bp, Artificial sequence
  Synthetic antibody
ggattcgacttcagtagctactactac

SEQ ID NO: 440 - DNA, 24 bp, Artificial sequence
  Synthetic antibody
attgatactggtagtggtgagact

SEQ ID NO: 441 - DNA, 30 bp, Artificial sequence
  Synthetic antibody
gcgagagatccctattttggcactaactta

SEQ ID NO: 442 - DNA, 24 bp, Artificial sequence
  Synthetic antibody
cagagtgtttataacaacaactac

SEQ ID NO: 443 - DNA, 33 bp, Artificial sequence
  Synthetic antibody
ctaggcggttatagtagtcctagtgatgatgct

SEQ ID NO: 444 - DNA, 27 bp, Artificial sequence
  Synthetic antibody
ggattctccttcagtagcagccactat

SEQ ID NO: 445 - DNA, 24 bp, Artificial sequence
  Synthetic antibody
atttatcttggtgatgacagcaca

SEQ ID NO: 446 - DNA, 42 bp, Artificial sequence
  Synthetic antibody
gcgagacatggatataatcttgctattcataattatattata

SEQ ID NO: 447 - DNA, 18 bp, Artificial sequence
  Synthetic antibody
gagagtagtagtcgtcac

SEQ ID NO: 448 - DNA, 42 bp, Artificial sequence
  Synthetic antibody
caaagctattatgggcttagtaataatggttggagatatgct

SEQ ID NO: 449 - DNA, 27 bp, Artificial sequence
  Synthetic antibody
ggattctccttcagtagcgactacgac

SEQ ID NO: 450 - DNA, 27 bp, Artificial sequence
  Synthetic antibody
attgatggtggtagtagtggtgagatt

SEQ ID NO: 451 - DNA, 36 bp, Artificial sequence
  Synthetic antibody
gcgagatgtatttggagtcataattactatgacttg

SEQ ID NO: 452 - DNA, 18 bp, Artificial sequence
  Synthetic antibody
gaggacatcgaaaactat

SEQ ID NO: 453 - DNA, 36 bp, Artificial sequence
  Synthetic antibody
caacagggttatagtggtagtaatgttgataatttt

SEQ ID NO: 454 - DNA, 27 bp, Artificial sequence
  Synthetic antibody
ggattctccttcagtagcagctactgg

SEQ ID NO: 455 - DNA, 27 bp, Artificial sequence
  Synthetic antibody
atttatcctagtagtagtggtagcact

SEQ ID NO: 456 - DNA, 51 bp, Artificial sequence
  Synthetic antibody
gcgagagatattccttataatattcatggttatactaccggatttaacttg

SEQ ID NO: 457 - DNA, 18 bp, Artificial sequence
  Synthetic antibody
cagagcattggtagtaat

SEQ ID NO: 458 - DNA, 36 bp, Artificial sequence
  Synthetic antibody
caatttacttattatggtagtagtgatgtcgatgtt

SEQ ID NO: 459 - DNA, 27 bp, Artificial sequence
  Synthetic antibody
ggattctccttcagtagcggctactgg

SEQ ID NO: 460 - DNA, 24 bp, Artificial sequence
  Synthetic antibody
attggtgctagttatggtggcact

SEQ ID NO: 461 - DNA, 36 bp, Artificial sequence
  Synthetic antibody
acgagagatgatggtggtcttgataatcctaatttg

SEQ ID NO: 462 - DNA, 18 bp, Artificial sequence
  Synthetic antibody
cagagcattggtagtatt

SEQ ID NO: 463 - DNA, 42 bp, Artificial sequence
  Synthetic antibody
caaagctattattatagtgttaccactagttattcgaatagt

SEQ ID NO: 464 - DNA, 27 bp, Artificial sequence
  Synthetic antibody
ggattcaccatcagtagtgactactac

SEQ ID NO: 465 - DNA, 27 bp, Artificial sequence
  Synthetic antibody
atttatattggtagtggtactgatact

SEQ ID NO: 466 - DNA, 48 bp, Artificial sequence
  Synthetic antibody
gcgagagataagggatggtctaatgcctggggtttctactttcagttg

SEQ ID NO: 467 - DNA, 18 bp, Artificial sequence
  Synthetic antibody
cagagcattggtaaatac

SEQ ID NO: 468 - DNA, 36 bp, Artificial sequence
  Synthetic antibody
caacagggttatagttatgttgatcttgaaaatggt

SEQ ID NO: 469 - DNA, 27 bp, Artificial sequence
  Synthetic antibody
ggattctccttcagtagcagctactgg

SEQ ID NO: 470 - DNA, 27 bp, Artificial sequence
  Synthetic antibody
atttatgctggtggtagtggtagcact

SEQ ID NO: 471 - DNA, 42 bp, Artificial sequence
  Synthetic antibody
gcgagagatggtgttagtggtagtggttggttctttaacttg

SEQ ID NO: 472 - DNA, 18 bp, Artificial sequence
  Synthetic antibody
cagagcattagtagctac

SEQ ID NO: 473 - DNA, 42 bp, Artificial sequence
  Synthetic antibody
caaagtagttattatggtcctagtgatgttgatttttctcct

SEQ ID NO: 474 - DNA, 27 bp, Artificial sequence
  Synthetic antibody
ggattctccttcagtaacaactactgg

SEQ ID NO: 475 - DNA, 27 bp, Artificial sequence
  Synthetic antibody
atttatgctggtgatactggtaccgct

SEQ ID NO: 476 - DNA, 45 bp, Artificial sequence
  Synthetic antibody
gcgagagatggggatggtgttgggaatggtggtacttttgccttg

SEQ ID NO: 477 - DNA, 18 bp, Artificial sequence
  Synthetic antibody
gagaggattggcaatgca

SEQ ID NO: 478 - DNA, 36 bp, Artificial sequence
  Synthetic antibody
caaagctattattatactggcagtattagttatgaa

SEQ ID NO: 479 - DNA, 27 bp, Artificial sequence
  Synthetic antibody
ggattctccttcagtagcagctactac

SEQ ID NO: 480 - DNA, 24 bp, Artificial sequence
  Synthetic antibody
atttttactggtggtgttagcacg

SEQ ID NO: 481 - DNA, 48 bp, Artificial sequence
  Synthetic antibody
gcgagaacctatactgctggttttactggtgataactactttaacttg

SEQ ID NO: 482 - DNA, 18 bp, Artificial sequence
  Synthetic antibody
cagagtattagtagttac

SEQ ID NO: 483 - DNA, 42 bp, Artificial sequence
  Synthetic antibody
caaagtgattattatagtagtagtagcaataatggttttgtt

SEQ ID NO: 484 - DNA, 27 bp, Artificial sequence
  Synthetic antibody
ggattctccttcagtggcccctactgg

SEQ ID NO: 485 - DNA, 27 bp, Artificial sequence
  Synthetic antibody
attggtgctcgtagtagtggtagcact

SEQ ID NO: 486 - DNA, 57 bp, Artificial sequence
  Synthetic antibody
gcgagatcgtatcctgcttatgctggtgacggttatgccctatactactttaacttg

SEQ ID NO: 487 - DNA, 18 bp, Artificial sequence
  Synthetic antibody
gaagacatttatagttta

SEQ ID NO: 488 - DNA, 39 bp, Artificial sequence
  Synthetic antibody
caaggcggttattatagtggtgatggtactgatatacct